(19) United States
(12) Patent Application Publication
(10) Pub. No.: US 2017/0114021 A1
(43) Pub. Date: Apr. 27, 2017

(54) HALOGEN-SUBSTITUTED COMPOUNDS

(71) Applicant: BAYER ANIMAL HEALTH GMBH, Leverkusen (DE)

(72) Inventors: Michael MAUE, Langenfeld (DE); Anne DECOR, Langenfeld (DE); Thomas BRETSCHNEIDER, Lohmar (DE); Julia Johanna HAHN, Duesseldorf (DE); Werner HALLENBACH, Monheim (DE); Reiner FISCHER, Monheim (DE); Hans-Georg SCHWARZ, Dorsten (DE); Ulrich GOERGENS, Ratingen (DE); Kerstin ILG, Koeln (DE); Klaus RAMING, Leverkusen (DE); Johannes KOEBBERLING, Neuss (DE); Walter HUEBSCH, Wuppertal (DE); Andreas TURBERG, Haan (DE)

(73) Assignee: BAYER ANIMAL HEALTH GMBH, Leverkusen (DE)

(21) Appl. No.: 15/318,930

(22) PCT Filed: Jun. 15, 2015
(86) PCT No.: PCT/EP2015/063277

§ 371 Date: Dec. 14, 2016

(30) Foreign Application Priority Data
Jun. 18, 2014 (EP) 14172922.8

(51) Int. Cl. C07D 231/12; A01N 43/713; C07D 403/04; A01N 43/56; A01N 43/647

(52) U.S. Cl. CPC C07D 231/12; A01N 43/56; A01N 43/647; C07D 403/04; A01N 43/713

(57) ABSTRACT

The invention encompasses inter alia halogen-substituted compounds of the general formula (I) in which the radicals A₁-A₄, T, W, Q, R¹ and Z¹-Z³ have the meanings given in the description. Also described are processes for preparing the compounds of the formula (I). The compounds according to the invention are especially suitable for controlling insects, arachnids and nematodes in agriculture, and ectoparasites in veterinary medicine.

The present application relates to novel halogen-substituted compounds, to processes for their preparation and to their use for controlling animal pests, in particular arthropods and especially insects, arachnids and nematodes.

It is known that certain halogen-substituted compounds have B-Raf-inhibiting activities for the treatment of cancer (WO 2009/003999). Furthermore, certain halogen-substituted compounds are described to have cytokine-inhibiting (WO 2005/090333) and protein tyrosine phosphatase-inhibiting (US 2004/0167188) properties.

Modern crop protection compositions have to meet many demands, for example in relation to efficacy, persistence and spectrum of action, and possible use. Questions of toxicity and of combinability with other active compounds or formulation auxiliaries play a role, as does the question of the expense that the synthesis of an active compound requires. In addition, resistances can occur. For all these reasons, the search for novel crop protection compositions can never be considered to be complete, and there is a constant need for novel compounds having improved properties compared to the known compounds at least in relation to individual aspects.

It was an object of the present invention to provide compounds which widen the spectrum of the pesticides in various aspects and/or improve their activity.

Surprisingly, it has now been found that certain halogen-substituted compounds and their N-oxides and salts have biological properties and are particularly suitable for controlling animal pests, and can therefore be employed particularly well in the agrochemical field and in the animal health sector.

### ABSTRACT

One aspect of the present invention relates to compounds of the general formula (I)

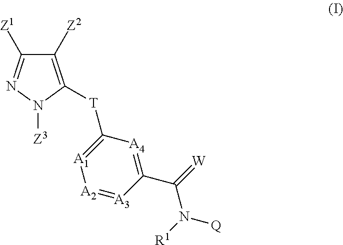

in which
R¹ represents hydrogen or a group, optionally substituted by at least one radical M¹, selected from the group consisting of (C₁-C₆)-alkyl, (C₂-C₆)-alkenyl, (C₂-C₆)-alkynyl, (C₃-C₇)-cycloalkyl, (C₁-C₆)-alkylcarbonyl, (C₁-C₆)-alkoxycarbonyl, aryl-(C₁-C₃)-alkyl and heteroaryl-(C₁-C₃)-alkyl;
the chemical moieties
A₁ represents CR² or nitrogen,
A₂ represents CR³ or nitrogen,
A₃ represents CR⁴ or nitrogen, and
A₄ represents CR⁵ or nitrogen;
where not more than three of the chemical moieties A₁ to A₄ simultaneously represent nitrogen;
R², R³, R⁴ and R⁵ independently of one another represent hydrogen, halogen, cyano, amino, nitro, or a group, optionally substituted by at least one radical M¹, selected from the group consisting of (C₁-C₆)-alkyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkylamino,
   if neither of the A₂ and A₃ moieties is nitrogen, R³ and R⁴ together with the carbon atom to which they are bonded may form a 5- or 6-membered ring containing 0, 1 or 2 nitrogen atoms and/or 0 or 1 oxygen atom and/or 0 or 1 sulphur atom, or
   if neither of the A₁ and A₂ moieties represents nitrogen, R² and R³ together with the carbon atom to which they are bonded may form a 6-membered ring containing 0, 1 or 2 nitrogen atoms;
W represents oxygen or sulphur;
Q represents hydrogen, hydroxy, or a group, optionally substituted by at least one radical M¹, selected from the group consisting of amino, N—(C₁-C₆)-alkylamino, N—(C₁-C₆)-alkylcarbonylamino, N,N-di-(C₁-C₆)-alkylamino, (C₁-C₆)-alkyl, (C₁-C₆)-alkoxy, (C₂-C₆)-alkenyl, (C₂-C₆)-alkynyl, (C₃-C₆)-cycloalkyl, heterocycloalkyl having 3 to 9 ring atoms, (C₁-C₆)-cycloalkyl-(C₁-C₆)-alkyl, (C₆)-aryl-(C₁-C₆)-alkyl, heteroaryl-(C₁-C₆)-alkyl having 5 to 7 ring atoms; or
Q represents an unsaturated 6-membered carbocycle which is optionally mono- or polysubstituted by V or represents an unsaturated 5- or 6-membered heterocyclic ring which is optionally mono- or polysubstituted by V, where
V independently of one another represent halogen, cyano, amino, nitro or a group, optionally substituted by at least one radical M¹, selected from the group consisting of (C₁-C₆)-alkyl, (C₂-C₄)-alkenyl, (C₂-C₄)-alkynyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkyl)amino;
T represents one of the 5-membered heteroaromatics T1-T8 listed below, where the bond to the pyrazole head group [C₃N₂Z₁Z₂Z₃] is marked with an asterisk *,

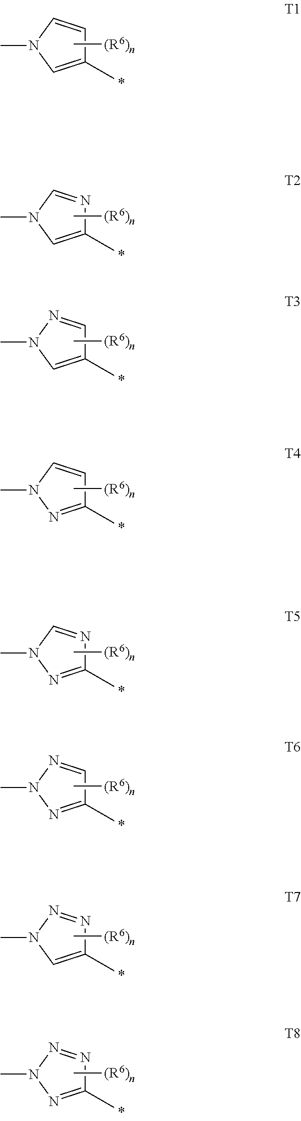

where
R⁶ independently of one another represent halogen, cyano, nitro or a group, optionally substituted by at least one radical M¹, selected from the group consisting of amino, (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkyl)amino;
n represents 0, 1 or 2; Z¹ represents a group, optionally substituted by at least one radical M¹, selected from the group consisting of (C₁-C₆)-alkyl, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl, preferably represents (C₁-C₆)-alkyl optionally substituted by at least one radical M¹;
Z² represents hydrogen, halogen, cyano, nitro or a group, optionally substituted by at least one radical M¹, selected from the group consisting of (C₁-C₆)-alkyl, (C₁-C₆)-alkylcarbonyl, (C₁-C₆)-alkylsulphanyl, (C₁-C₆)-alkylsulphinyl and (C₁-C₆)-alkylsulphonyl;
Z³ represents hydrogen or a group, optionally substituted by at least one radical M¹, selected from the group consisting of (C₁-C₆)-alkyl, (C₁-C₆)-cycloalkyl, (C₁-C₆)-alkenyl, (C₁-C₆)-alkynyl, (C₆)-aryl and hetaryl having 5 or 6 ring atoms;
M¹ represents halogen, cyano, isocyanato, azido, hydroxy, nitro, formyl, carboxyl or a group equivalent to the carboxyl group, or a group, optionally substituted by at least one radical M², selected from the group consisting of amino, (C₁-C₄)-alkyl, (C₁-C₄)-haloalkyl, (C₁-C₄)-alkoxy, (C₁-C₄)-haloalkoxy, (C₁-C₄)-alkoxy-(C₁-C₄)-alkoxy, (C₁-C₄)-alkoxy-(C₁-C₄)-alkyl, (C₁-C₄)-alkylsulphanyl, (C₁-C₄)-haloalkylsulphanyl, (C₁-C₄)-alkoxycarbonyl, (C₁-C₄)-alkylcarbonyl, carbamoyl, mono- and —N,N-di-(C₁-C₄)-alkylaminocarbonyl, (C₁-C₄)-acylamino, mono- and N,N-di-(C₁-C₄)-alkylamino, tri-(C₁-C₄)-alkylsilyl, (C₃-C₆)-cycloalkyl, C₆-aryl, heterocyclyl having 3 to 6 ring atoms, where each of the last-mentioned cyclic groups may also be attached via heteroatoms or a divalent functional —CH₂— or —C₂H₄— group, (C₁-C₄)-alkylsulphinyl, where both enantiomers of the (C₁-C₄)-alkylsulphonyl group are included, (C₁-C₄)-alkylsulphonyl, (C₁-C₄)-alkylphosphinyl, (C₁-C₄)-alkylsulphanyl-(C₁-C₄)-alkyl, (C₁-C₄)-alkoxy-(C₁-C₄)-alkyl, mono- and N,N-di-(C₁-C₄)-alkylamino-(C₁-C₄)-alkyl and hydroxy-(C₁-C₄)-alkyl; and
M² represents amino, hydroxy, halogen, nitro, cyano, isocyanato, mercapto, isothiocyanato, carboxyl or carboxamide.

A preferred embodiment relates to compounds of the formula (I) in which Z¹ and Z² independently of one another represent perhalogenated (C₁-C₄)-alkyl and Z³ represents (C₁-C₄)-alkyl and the other parameters are as defined above.

A further preferred embodiment relates to compounds of the formula (I) in which Z¹ represents perfluorinated ethyl (C₂F₅), Z² represents perfluorinated methyl (CF₃) and Z³ represents methyl and the other parameters are as defined above.

A further preferred embodiment relates to compounds of the formula (I) in which T represents T1 (formula (Ia)), T6 (formula (If)), T7 (formula (Ig)) or T8 (formula (Ih)) and the other parameters are as defined above.

A further preferred embodiment relates to compounds of the formula (I) in which T represents T3 (formula (Ic)), T6 (formula (If)), T7 (formula (Ig)) or T8 (formula (Ih)) and the other parameters are as defined above.

A further preferred embodiment relates to compounds of the formula (I) in which Q represents hydrogen, (C₁-C₆)-alkyl, (C₃-C₆)-cycloalkyl, (C₁-C₆)-alkoxy or benzyl and the other parameters are as defined above.

A further preferred embodiment relates to compounds of the formula (I) in which n in a structure T1 to T8 has the value 0 and the other parameters are as defined above.

A further preferred embodiment relates to compounds of the formula (I) in which
T represents T3 (formula (Ic)), T6 (formula (If)), T7 (formula (Ig)) or T8 (formula (Ih)) and n in T represents 0;
A₁ represents CR₂, where R₂ represents hydrogen;
A₂ represents CR₃, where R₃ represents hydrogen;
A₃ represents CR₄, where R₄ represents hydrogen, halogen, (C₁-C₄)-alkyl or (C₁-C₄)-alkoxy, preferably halogen selected from the group consisting of chlorine, fluorine, bromine and iodine;
A₄ represents CR₅, where R₅ represents hydrogen;
W represents oxygen;
n in T represents 0;
Z¹ represents halogenated (C₁-C₄)-alkyl;
Z² represents halogenated (C₁-C₄)-alkyl;
Z³ represents (C₁-C₄)-alkyl;
R₁ represents hydrogen or (C₁-C₄)-alkyl; preferably hydrogen; and
Q represents hydrogen or a group, optionally substituted by at least one radical M¹, selected from the group consisting of (C₁-C₆)-alkyl and (C₃-C₆)-cycloalkyl, where M¹ independently of one another represent cyano, chlorine, bromine, iodine or fluorine.

A further preferred embodiment relates to compounds of the formula (I) in which
T represents T3 (formula (Ic)), T6 (formula (If)), T7 (formula (Ig)) or T8 (formula (Ih)) and n in T represents 0;
A₁ represents CR₂, where R₂ represents hydrogen;
A₂ represents CR₃, where R₃ represents hydrogen;
A₃ represents CR₄, where R₄ represents chlorine;
A₄ represents CR₅, where R₅ represents hydrogen;
W represents oxygen;
n in T represents 0;
Z¹ represents perfluorinated ethyl;
Z² represents perfluorinated methyl;
Z³ represents methyl;
R₁ represents hydrogen or (C₁-C₄)-alkyl; preferably hydrogen;
Q represents (C₃-C₆)-cycloalkyl, preferably cyclopropyl.

A further aspect of the present invention relates to the use of a compound of the formula (I) as defined in the present application for controlling insects, arachnids and nematodes.

A further aspect of the present invention relates to a pharmaceutical composition comprising at least one compound of the formula (I) as defined in the present application.

A further aspect of the present invention relates to a compound of the formula (I) as defined in the present application for use as a medicament.

A further aspect of the present invention relates to the use of a compound of the formula (I) as defined in the present application for preparing pharmaceutical compositions for controlling parasites on animals.

A further aspect of the present invention relates to a process for preparing crop protection compositions comprising a compound of the formula (I) as defined in the present application and customary extenders and/or surfactants.

A further aspect of the present invention relates to the use of a compound of the formula (I) as defined in the present application for protecting the propagation material of plants, preferably for protecting seed.

### DEFINITIONS

The person skilled in the art is aware that the expressions “a” or “an” as used in the present application may, depending on the situation, mean “one (1)”, “one (1) or more” or “at least one (1)”.

The expression “optionally substituted” means, if no specific substituents are stated, that the group in question may be mono- or polysubstituted by a substituent M¹, where in the case of polysubstitution the substituents M¹ can be identical or different.

It will be clear to the person skilled in the art that examples cited in the present application should not be considered in a restrictive manner, but merely describe some embodiments in detail.

In the definitions of the symbols given in the above formulae, collective terms which are generally representative of the following substituents were used:

According to the invention, “alkyl”—on its own or as a part of a chemical group—represents straight-chain or branched hydrocarbons having preferably 1 to 6 carbon atoms, particularly preferably 1, 2, 3 or 4 carbon atoms, for example methyl, ethyl, n-propyl, isopropyl, n-butyl, isobutyl, s-butyl, t-butyl. The alkyl radicals according to the invention may optionally be substituted by one or more identical or different radicals M¹.

According to the invention, “alkenyl”—on its own or as a part of a chemical group—represents straight-chain or branched hydrocarbons having preferably 2 to 6 carbon atoms, particularly preferably 2, 3 or 4 carbon atoms, and at least one double bond, for example vinyl, 2-propenyl, 2-butenyl, 3-butenyl, 1-methyl-2-propenyl, 2-methyl-2-propenyl, 2-pentenyl, 3-pentenyl, 4-pentenyl, etc. The alkenyl radicals according to the invention may optionally be substituted by one or more identical or different radicals M¹.

According to the invention, “alkynyl”—on its own or as a part of a chemical group—represents straight-chain or branched hydrocarbons having preferably 2 to 6 carbon atoms, particularly preferably 2, 3 or 4 carbon atoms, and at least one triple bond, for example ethynyl, 2-propynyl, 2-butynyl, 3-butynyl, 1-methyl-2-propynyl, 2-pentynyl, 3-pentynyl, 4-pentynyl, 1-methyl-3-butynyl, etc. The alkynyl radicals according to the invention may optionally be substituted by one or more identical or different radicals M¹.

According to the invention, “cycloalkyl”—on its own or as part of a chemical group—represents mono-, bi- or tricyclic hydrocarbons preferably having 3 to 10 carbons, for example cyclopropyl, cyclobutyl, cyclopentyl, cyclohexyl, bicyclo[2.2.1]heptyl, bicyclo[2.2.2]octyl or adamantyl, particularly preferably cycloalkyl radicals having 3, 4, 5, 6 or 7 carbon atoms, for example cyclopropyl or cyclobutyl. The cycloalkyl radicals according to the invention may optionally be substituted by one or more identical or different radicals M¹.

According to the invention, “alkylcycloalkyl” represents mono-, bi- or tricyclic alkylcycloalkyl preferably having 4 to 10 or 4 to 7 carbon atoms, particularly preferably alkylcycloalkyl radicals having 4, 5 or 7 carbon atoms, for example ethylcyclopropyl or 4-methylcyclohexyl, where the alkylcycloalkyl is attached via the cycloalkyl moiety to the parent structure. The alkylcycloalkyl radicals according to the invention may optionally be substituted by one or more identical or different radicals M¹.

According to the invention, “cycloalkylalkyl” represents mono-, bi- or tricyclic cycloalkylalkyl preferably having 4 to 10 or 4 to 7 carbon atoms, particularly preferably cycloalkylalkyl radicals having 4, 5 or 7 carbon atoms, inter alia cyclopropylmethyl or cyclobutylmethyl, where the cycloalkylalkyl is attached via the alkyl moiety to the parent structure. The cycloalkylalkyl radicals according to the invention may optionally be substituted by one or more identical or different radicals M¹.

According to the invention, “alkoxy” represents a straight-chain or branched O-alkyl preferably having 1 to 6 carbon atoms, more preferably alkoxy groups having 1 to 4 carbon atoms, for example methoxy, ethoxy, n-propoxy, isopropoxy, n-butoxy, isobutoxy, s-butoxy or t-butoxy. The alkoxy groups according to the invention may optionally be substituted by one or more identical or different radicals M¹.

According to the invention, “alkylsulphanyl” represents straight-chain or branched S-alkyl preferably having 1 to 6 carbon atoms, more preferably alkylsulphanyl groups having 1 to 4 carbon atoms, for example methylthio, ethylthio, n-propylthio, isopropylthio, n-butylthio, isobutylthio, s-butylthio and t-butylthio. The alkylsulphanyl groups according to the invention may optionally be substituted by one or more identical or different radicals M¹.

According to the invention, “alkylsulphinyl” represents straight-chain or branched alkylsulphinyl preferably having 1 to 6 carbon atoms, more preferably alkylsulphinyl groups having 1 to 4 carbon atoms, for example methylsulphinyl, ethylsulphinyl, n-propylsulphinyl, isopropylsulphinyl, n-butylsulphinyl, isobutylsulphinyl, s-butylsulphinyl and t-butylsulphinyl. The alkylsulphinyl groups according to the invention may optionally be substituted by one or more identical or different radicals M¹.

According to the invention, “alkylsulphonyl” represents straight-chain or branched alkylsulphonyl preferably having 1 to 6 carbon atoms, more preferably alkylsulphonyl groups having 1 to 4 carbon atoms, for example methylsulphonyl, ethylsulphonyl, n-propylsulphonyl, isopropylsulphonyl, n-butylsulphonyl, isobutylsulphonyl, s-butylsulphonyl and t-butylsulphonyl. The alkylsulphonyl groups according to the invention may optionally be substituted by one or more identical or different radicals M¹.

According to the invention, “acyl” represents radicals containing an X¹—C(═O)—X² group, where X¹ and X² independently of one another represent an organic radical as defined in the present application or represent hydrogen or represent a bond to the parent structure of a compound of the formula (I). In particular, “acyl” is understood to mean acids, esters, aldehydes, alkylcarbonyl (alkyl-C(═O)—) and amides. Preferably, X¹ and X² each independently of one another represent a group, optionally substituted by one or more identical or different radicals M¹, selected from alkyl, alkylene (—CₙH₂ₙ—), alkoxy, alkoxylene (—O—CₙH₂ₙ—), amino, mono- or dialkylamino, or hydrogen, or a radical X¹ or X² represents a bond to the parent structure of a compound of the formula (I).

According to the invention “alkylcarbonyl” represents straight-chain or branched alkyl-C(═O)— preferably having 2 to 7 carbon atoms (including the carbon atom of the C(═O) group), more preferably alkylcarbonyl groups having 2 to 5 carbon atoms ((C₁-C₄)-alkyl-C(═O)—), such as methylcarbonyl, ethylcarbonyl, n-propylcarbonyl, isopropylcarbonyl, s-butylcarbonyl and t-butylcarbonyl. The alkylcarbonyl groups according to the invention may optionally be substituted by one or more identical or different radicals M¹.

According to the invention, “cycloalkylcarbonyl” represents straight-chain or branched cycloalkylcarbonyl preferably having 3 to 10 carbon atoms in the cycloalkyl moiety, more preferably cycloalkylcarbonyl having 3, 5 or 7 carbon atoms in the cycloalkyl moiety, for example cyclopropylcarbonyl, cyclobutylcarbonyl, cyclopentylcarbonyl, cyclohexylcarbonyl, cycloheptylcarbonyl, cyclooctylcarbonyl, bicyclo[2.2.1]heptyl, bicyclo[2.2.2]octylcarbonyl and adamantylcarbonyl. The cycloalkylcarbonyl groups according to the invention may optionally be substituted by one or more identical or different radicals M¹.

According to the invention, “alkoxycarbonyl”—on its own or as part of a chemical group—represents straight-chain or branched alkoxycarbonyl preferably having 1 to 6 carbon atoms, more preferably having 1, 2, 3 or 4 carbon atoms in the alkoxy moiety, for example methoxycarbonyl, ethoxycarbonyl, n-propoxycarbonyl, isopropoxycarbonyl, s-butoxycarbonyl and t-butoxycarbonyl. The alkoxycarbonyl groups according to the invention may optionally be substituted by one or more identical or different radicals M¹.

According to the invention, “halogen” represents fluorine (F), chlorine (Cl), bromine (Br) or iodine (I).

The expressions “haloalkyl”, “haloalkenyl”, “haloalkynyl”, “haloalkylcarbonyl” “haloalkoxy”, “haloalkoxycarbonyl”, “haloalkylsulphanyl”, “haloalkylsulphinyl” or “haloalkylsulphonyl” as used herein refer to a chemical alkyl, alkenyl, alkynyl, alkylcarbonyl, alkoxy, alkoxycarbonyl, alkylsulphanyl, alkylsulphinyl or alkylsulphonyl group (in each case preferably having one to 6 carbon atoms or more preferably having one, two, three or four carbon atoms) substituted by at least one halogen. The halogen groups may be mono- to polysubstituted up to the maximum possible number of substituents (perhalogenated) by halogen. In the case of polysubstitution by halogen, the halogen atoms may be identical or different, and may all be bonded to one carbon atom or may be bonded to a plurality of carbon atoms. Here, halogen represents in particular fluorine, chlorine, bromine or iodine, preferably fluorine or chlorine and more preferably fluorine. In a preferred embodiment, perhalogenated groups are maximally substituted by only one type of halogen, e.g. perfluorinated methyl (trifluoromethyl; CF₃) or perfluorinated ethyl (pentafluoroethyl; C₂F₅). Some examples of “haloalkyl”, “haloalkenyl”, “haloalkynyl”, “haloalkylcarbonyl”, “haloalkoxy”, “haloalkoxycarbonyl”, “haloalkylsulphanyl”, “haloalkylsulphinyl” or “haloalkylsulphonyl” are trichloromethyl (CCl₃), trifluoromethyl (CF₃), chlorodifluoromethyl (CClF₂), dichlorofluoromethyl (CCl₂F), 2,2-difluoroethyl (F₂HCCH₂), 2,2,2-trifluoroethyl (F₃CCH₂), pentafluoroethyl (C₂F₅), 2,2-difluoroethenyl (CHCF₂), 2-chloroethynyl (CHCCl), trifluoromethoxy —OCF₃, difluoromethoxy —OCHF₂, 1,1,2,2-tetrafluoroethylthio, 2-chloro-1,1,2-trifluoroethylsulphinyl, trichloromethylsulphonyl, etc. The halo groups according to the invention can optionally be substituted by one or more identical or different radicals M¹, provided at least one hydrogen atom at a carbon atom of the halo group is replaced by a halogen. An example of an M¹-substituted haloalkyl is 2-cyano-2,2-difluoroethyl (C(CN)F₂CH₂).

An amino group (—NH₂) may optionally be substituted by one or more identical or different radicals M¹.

Substituted amino such as mono- or disubstituted amino means a radical from the group of the substituted amino radicals which are N-substituted, for example, by one or two identical or different radicals from the group consisting of alkyl, hydroxy, amino, alkoxy, acyl and aryl; preferably N-mono- and N,N-dialkylamino, (for example methylamino, ethylamino, N,N-dimethylamino, N,N-diethylamino, N,N-di-n-propylamino, —N,N-diisopropylamino or —N,N-dibutylamino), —N-mono- or —N,N-dialkoxyalkylamino groups (for example N-methoxymethylamino, N-methoxyethylamino, N,N-di(methoxymethyl)amino or N,N-di(methoxyethyl)amino), N-mono- and N,N-diarylamino, such as optionally substituted anilines, acylamino, N,N-diacylamino, N-alkyl-N-arylamino, N-alkyl-N-acylamino and also saturated N-heterocycles; preference is given here to alkyl radicals having 1 to 4 carbon atoms; here, aryl is preferably phenyl or optionally substituted phenyl; for acyl, the definition given further above applies, preferably (C₁-C₄)-alkyl-C(═O)—.

Substituted amino also includes quaternary ammonium compounds (salts) with four organic substituents on the nitrogen atom.

According to the invention, “hydroxyalkyl” represents a straight-chain or branched alcohol preferably having 1 to 6 carbon atoms, more preferably 1, 2, 3 or 4 carbon atoms, for example methanol, ethanol, n-propanol, isopropanol, n-butanol, isobutanol, s-butanol and t-butanol. The hydroxyalkyl groups according to the invention may be substituted by one or more identical or different radicals M¹.

According to the invention, “alkylaminocarbonyl” represents straight-chain or branched alkylaminocarbonyl having preferably 1 to 6 carbon atoms, more preferably 1, 2, 3 or 4 carbon atoms in the alkyl moiety, for example methylaminocarbonyl (—CONHCH₃), ethylaminocarbonyl, n-propylaminocarbonyl, isopropylaminocarbonyl, s-butylaminocarbonyl and t-butylaminocarbonyl. The alkylaminocarbonyl groups according to the invention may optionally be substituted by one or more identical or different radicals M¹.

According to the invention, “N,N-dialkylaminocarbonyl” (—C(═O)N(alkyl)₂) represents straight-chain or branched N,N-dialkylaminocarbonyl having preferably 1 to 6 carbon atoms per alkyl, more preferably 1, 2, 3 or 4 carbon atoms per alkyl, for example N,N-dimethylaminocarbonyl (—C(═O)N(CH₃)₂), —N,N-diethylaminocarbonyl, —N,N-di(n-propylamino)carbonyl, —N,N-di(isopropylamino)carbonyl and N,N-di(s-butylamino)carbonyl. The N,N-dialkylaminocarbonyl groups according to the invention may optionally be substituted by one or more identical or different radicals M¹.

“Carbocycle”, unless defined differently elsewhere, is in particular cycloalkyl, cycloalkenyl or aryl. A carbocycle is in particular mono-, bi- or tricyclic C₆- to C₁₄-aryl. A carbocycle may optionally be substituted by one or more identical or different radicals M¹.

According to the invention, “aryl” represents a mono-, bi- or polycyclic aromatic system having preferably 6 to 14, in particular 6 to 10, ring carbon atoms, for example phenyl, naphthyl, anthryl, phenanthrenyl, preferably phenyl. In addition, aryl also represents polycyclic systems such as tetrahydronaphthyl, indenyl, indanyl, fluorenyl, biphenyl, where the bonding site is on the aromatic system. The aryl groups according to the invention may optionally be substituted by one or more identical or different radicals M¹.

According to the invention, “arylalkyl” represents an aryl-substituted alkyl radical having preferably 6 to 14, in particular 6 to 10 ring carbon atoms in the aryl moiety and 1 to 6, in particular 1 to 4 carbon atoms in the alkyl moiety. Arylalkyl may be substituted by one or more identical or different radicals in the alkyl and/or aryl moiety. Examples of such arylalkyls include benzyl and 1-phenylethyl. The arylalkyl groups according to the invention may optionally be substituted by one or more identical or different radicals M¹.

According to the invention, “heterocycle”, “heterocyclic ring” or “heterocyclic ring system” represents a carbocyclic ring system having at least one ring in which at least one carbon atom is replaced by a heteroatom, preferably by a heteroatom from the group consisting of N, O, S, P, B, Si, Se, and which is saturated, unsaturated or heteroaromatic and may be unsubstituted or substituted by a substituent Z, where the point of attachment is located at a ring atom. Unless defined otherwise, the heterocyclic ring contains preferably 3 to 9 ring atoms, especially 3 to 6 ring atoms, and one or more, preferably 1 to 4, especially 1, 2 or 3, heteroatoms in the heterocyclic ring, preferably from the group of N, O and S, although no two oxygen atoms should be directly adjacent to one another. The heterocyclic rings usually contain not more than 4 nitrogen atoms and/or not more than 2 oxygen atoms and/or not more than 2 sulphur atoms. If the heterocyclyl radical or the heterocyclic ring is optionally substituted, it may be fused to other carbocyclic or heterocyclic rings. In the case of optionally substituted heterocyclyl, the invention also embraces polycyclic systems, for example 8-azabicyclo[3.2.1]octanyl or 1-azabicyclo[2.2.1]heptyl. In the case of optionally substituted heterocyclyl, the invention also embraces spirocyclic systems, for example 1-oxa-5-azaspiro[2.3]hexyl. The groups “heterocycle”, “heterocyclic ring” or “heterocyclic ring system” according to the invention may optionally be substituted by one or more identical or different radicals M¹.

Heterocyclyl groups according to the invention are, for example, piperidinyl, piperazinyl, morpholinyl, thiomorpholinyl, dihydropyranyl, tetrahydropyranyl, dioxanyl, pyrrolinyl, pyrrolidinyl, imidazolinyl, imidazolidinyl, thiazolidinyl, oxazolidinyl, dioxolanyl, dioxolyl, pyrazolidinyl, tetrahydrofuranyl, dihydrofuranyl, oxetanyl, oxiranyl, azetidinyl, aziridinyl, oxazetidinyl, oxaziridinyl, oxazepanyl, oxazinanyl, azepanyl, oxopyrrolidinyl, dioxopyrrolidinyl, oxomorpholinyl, oxopiperazinyl and oxepanyl.

Of particular significance are heteroaryls, i.e. heteroaromatic systems. According to the invention, the term heteroaryl represents heteroaromatic compounds, i.e. completely unsaturated aromatic heterocyclic compounds which fall under the above definition of heterocycles. Preference is given to 5- to 7-membered rings having 1 to 3, preferably 1 or 2, identical or different heteroatoms from the group above. Heteroaryls according to the invention are, for example, furyl, thienyl, pyrazolyl, imidazolyl, 1,2,3- and 1,2,4-triazolyl, isoxazolyl, thiazolyl, isothiazolyl, 1,2,3-, 1,3,4-, 1,2,4- and 1,2,5-oxadiazolyl, azepinyl, pyrrolyl, pyridyl, pyridazinyl, pyrimidinyl, pyrazinyl, 1,3,5-, 1,2,4- and 1,2,3-triazinyl, 1,2,4-, 1,3,2-, 1,3,6- and 1,2,6-oxazinyl, oxepinyl, thiepinyl, 1,2,4-triazolonyl and 1,2,4-diazepinyl. Furthermore, the heteroaryl groups according to the invention may optionally be substituted by one or more identical or different radicals M¹.

For the purpose of the present invention, “substituted” group or groups “substituted by at least one radical M¹” such as an optionally substituted alkyl, alkenyl, alkynyl, cycloalkyl, aryl, phenyl, benzyl, heterocyclyl, heteroaryl or amino radical, etc., is generally a group containing at least one hydrocarbon-containing or nitrogen-hydrogen-containing fraction where the hydrogen is replaced by a different atom or an atom group M¹. In other words, such a group is a substituted group derived from the unsubstituted parent structure, where the parent structure is substituted by one or more substituents M¹, preferably 1, 2 or 3 radicals M¹, and the substituent(s) M¹ is/are each independently of one another selected from the group consisting of halogen, hydroxy, nitro, formyl, carboxy, cyano, amino, isocyano, azido, (C₁-C₄)-alkyl, (C₁-C₄)-haloalkyl, (C₂-C₄)-alkenyl, (C₂-C₄)-alkynyl, (C₃-C₆)-cycloalkyl, (C₁-C₄)-alkoxy, (C₁-C₄)-haloalkoxy, (C₁-C₄)-alkoxy-(C₁-C₄)-alkoxy, (C₁-C₄)-alkoxy-(C₁-C₆)-alkyl, N—(C₁-C₄)-alkoxyimino-(C₁-C₃)-alkyl, (C₁-C₄)-alkylsulphanyl, (C₁-C₄)-haloalkylsulphanyl, (C₁-C₄)-alkoxycarbonyl, (C₁-C₄)-alkylcarbonyl, carbamoyl, C₁-C₄-alkylcarbamoyl, C₃-C₇-cycloalkylcarbamoyl, mono- and N,N-di(C₁-C₄)-alkylaminocarbonyl, amino, (C₁-C₆)-acylamino, mono- and N,N-di(C₁-C₄)-alkylamino, tri(C₁-C₄)-alkylsilyl, (C₃-C₆)-cycloalkyl, C₆-aryl, heterocyclyl having 3 to 6 ring atoms, where each of the last-mentioned cyclic groups may also be attached via heteroatoms or a divalent functional CH₂ or C₂H₄ group, (C₁-C₄)-alkylsulphinyl, where both enantiomers of the (C₁-C₄)-alkylsulphinyl group are included, (C₁-C₄)-alkylsulphonyl, (C₁-C₄)-alkylphosphinyl, (C₁-C₄)—(C₁-C₄)-alkylsulphanyl-(C₁-C₄)-alkyl, (C₁-C₄)-alkoxy-(C₁-C₄)-alkyl, mono- and N,N-di(C₁-C₄)-alkylamino-(C₁-C₄)-alkyl and hydroxy(C₁-C₄)-alkyl. The radicals M¹ mentioned in an exemplary manner can be unsubstituted or optionally (e.g. alkyl or amino), if they contain hydrocarbon-containing or nitrogen-hydrogen-containing fractions, substituted by one or more, preferably 1, 2 or 3 radicals M², where M² independently of the others is selected from the group consisting of amino, hydroxy, halogen, nitro, cyano, isocyano, mercapto, isothiocyanato, carboxy and carboxamide.

If two or more radicals form one or more rings, these may be carbocyclic, heterocyclic, saturated, partially saturated, unsaturated, for example also aromatic and further-substituted.

Optionally substituted phenyl is preferably phenyl which is unsubstituted or mono- or polysubstituted, preferably mono-, di- or trisubstituted, by identical of different radicals selected from the group consisting of halogen, cyano, isocyano, nitro; (C₁-C₄)-alkyl, C₂-C₄-alkenyl, C₂-C₄-alkynyl, (C₁-C₄)-alkoxy, (C₁-C₄)-alkoxy-(C₁-C₄)-alkoxy, (C₁-C₄)-alkoxy-(C₁-C₄)-alkyl, (C₃-C₆)-cycloalkyl, (C₁-C₄)-haloalkyl, (C₁-C₄)-haloalkoxy, (C₁-C₄)-alkylsulphanyl, (C₁-C₄)-haloalkylsulphanyl, optionally substituted by at least one radical M², for example o-, m- and p-tolyl, dimethylphenyl radicals, 2-, 3- and 4-chlorophenyl, 2-, 3- and 4-fluorophenyl, 2-, 3- and 4-trifluoromethyl and -trichloromethylphenyl, 2,4-, 3,5-, 2,5- and 2,3-dichlorophenyl, o-, m- and p-methoxyphenyl.

Optionally substituted cycloalkyl is preferably cycloalkyl, which is unsubstituted or mono- or polysubstituted, preferably up to trisubstituted, by identical or different radicals selected from the group consisting of halogen, haloalkyl, cyano, (C₁-C₄)-alkyl, (C₁-C₄)-alkoxy, (C₁-C₄)-alkoxy-(C₁-C₄)-alkoxy, (C₁-C₄)-alkoxy-(C₁-C₄)-alkyl, (C₁-C₄)-haloalkyl and (C₁-C₄)-haloalkoxy.

Optionally substituted heterocyclyl is preferably heterocyclyl which is unsubstituted or mono- or polysubstituted, preferably up to trisubstituted, by identical or different radicals from the group of halogen, cyano, (C₁-C₄)-alkyl, (C₁-C₄)-alkoxy, (C₁-C₄)-alkoxy-(C₁-C₄)-alkoxy, (C₁-C₄)-alkoxy-(C₁-C₄)-alkyl, (C₁-C₄)-haloalkyl, (C₁-C₄)-haloalkoxy, nitro and oxo, especially mono- or polysubstituted by radicals from the group of halogen, (C₁-C₄)-alkyl, (C₁-C₄)-alkoxy, (C₁-C₄)-haloalkyl and oxo, very particularly substituted by one or two (C₁-C₄)-alkyl radicals.

Examples of alkyl-substituted heteroaryls are furylmethyl, thienylmethyl, pyrazolylmethyl, imidazolylmethyl, 1,2,3- and 1,2,4-triazolylmethyl, isoxazolylmethyl, thiazolylmethyl, isothiazolylmethyl, 1,2,3-, 1,3,4-, 1,2,4- and 1,2,5-oxadiazolylmethyl, azepinylmethyl, pyrrolylmethyl, pyridylmethyl, pyridazinylmethyl, pyrimidinylmethyl, pyrazinylmethyl, 1,3,5-, 1,2,4- and 1,2,3-triazinylmethyl, 1,2,4-, 1,3,2-, 1,3,6- and 1,2,6-oxazinylmethyl, oxepinylmethyl, thiepinylmethyl and 1,2,4-diazepinylmethyl.

Not included are combinations which contravene the laws of nature and which the person skilled in the art would therefore rule out on the basis of his/her expert knowledge. Ring structures having three or more adjacent oxygen atoms, for example, are excluded.

### DETAILED DESCRIPTION

The halogen-substituted compounds according to the invention are defined by the general formula (I)

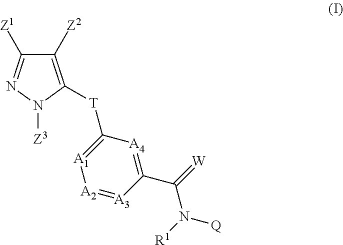

in which
R¹ represents hydrogen or a group, optionally substituted by at least one radical M¹, selected from the group consisting of (C₁-C₆)-alkyl, (C₂-C₆)-alkenyl, (C₂-C₆)-alkynyl, (C₃-C₇)-cycloalkyl, (C₁-C₆)-alkylcarbonyl, (C₁-C₆)-alkoxycarbonyl, aryl-(C₁-C₃)-alkyl and heteroaryl-(C₁-C₃)-alkyl;
the chemical moieties
A₁ represents CR² or nitrogen,
A₂ represents CR³ or nitrogen,
A₃ represents CR⁴ or nitrogen, and
A₄ represents CR⁵ or nitrogen;
where not more than three of the chemical moieties A₁ to A₄ simultaneously represent nitrogen;
R², R³, R⁴ and R⁵ independently of one another represent hydrogen, halogen, cyano, amino, nitro, or a group, optionally substituted by at least one radical M¹, selected from the group consisting of (C₁-C₆)-alkyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkylamino,
   if neither of the A₂ and A₃ moieties is nitrogen, R³ and R⁴ together with the carbon atom to which they are bonded may form a 5- or 6-membered ring containing 0, 1 or 2 nitrogen atoms and/or 0 or 1 oxygen atom and/or 0 or 1 sulphur atom, or
   if neither of the A₁ and A₂ moieties represents nitrogen, R² and R³ together with the carbon atom to which they are bonded may form a 6-membered ring containing 0, 1 or 2 nitrogen atoms;
W represents oxygen or sulphur;
Q represents hydrogen, hydroxy, or a group, optionally substituted by at least one radical M¹, selected from the group consisting of amino, N—(C₁-C₆)-alkylamino, N—(C₁-C₆)-alkylcarbonylamino, N,N-di-(C₁-C₆)-alkylamino, (C₁-C₆)-alkyl, (C₁-C₆)-alkoxy, (C₂-C₆)-alkenyl, (C₂-C₆)-alkynyl, (C₃-C₆)-cycloalkyl, heterocycloalkyl having 3 to 9 ring atoms, (C₁-C₆)-cycloalkyl-(C₁-C₆)-alkyl, (C₆)-aryl-(C₁-C₆)-alkyl, heteroaryl-(C₁-C₆)-alkyl having 5 to 7 ring atoms; or
Q represents an unsaturated 6-membered carbocycle which is optionally mono- or polysubstituted by V or represents an unsaturated 5- or 6-membered heterocyclic ring which is optionally mono- or polysubstituted by V, where
V independently of one another represent halogen, cyano, amino, nitro or a group, optionally substituted by at least one radical M¹, selected from the group consisting of (C₁-C₆)-alkyl, (C₂-C₄)-alkenyl, (C₂-C₄)-alkynyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkyl)amino;
T represents one of the 5-membered heteroaromatics T1-T8 listed below, where the bond to the pyrazole head group [C₃N₂Z₁Z₂Z₃] is marked with an asterisk *,

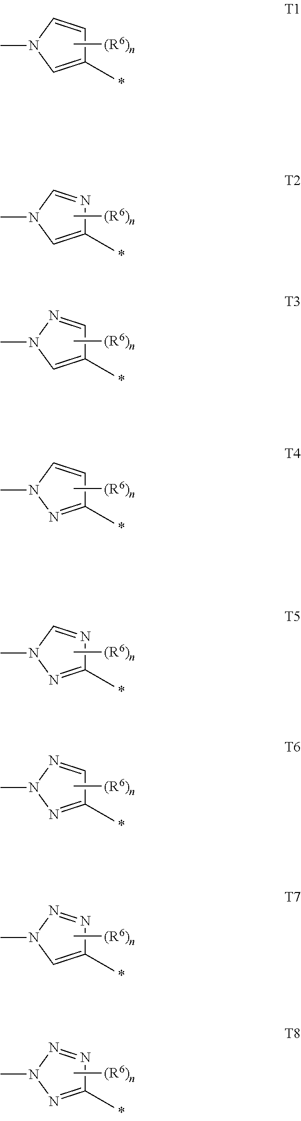

where
R⁶ independently of one another represent halogen, cyano, nitro or a group, optionally substituted by at least one radical M¹, selected from the group consisting of amino, (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl, N—(C₁-C₆)-alkylamino and —N,N-di-(C₁-C₆)-alkyl)amino;
n represents 0, 1 or 2;
Z¹ represents a group, optionally substituted by at least one radical M¹, selected from the group consisting of (C₁-C₆)-alkyl, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl-substituted (C₁-C₄)-haloalkyl, preferably represents (C₁-C₆)-alkyl optionally substituted by at least one radical M¹;
Z² represents hydrogen, halogen, cyano, nitro or a group, optionally substituted by at least one radical M¹, selected from the group consisting of (C₁-C₆)-alkyl, (C₁-C₆)-alkylcarbonyl, (C₁-C₆)-alkylsulphanyl, (C₁-C₆)-alkylsulphinyl and (C₁-C₆)-alkylsulphonyl;
Z³ represents hydrogen or a group, optionally substituted by at least one radical M¹, selected from the group consisting of (C₁-C₆)-alkyl, (C₃-C₆)-cycloalkyl, (C₃-C₆)-alkenyl, (C₁-C₆)-alkynyl, (C₆)-aryl and hetaryl having 5 or 6 ring atoms;
M¹ represents halogen, cyano, isocyanato, azido, hydroxy, nitro, formyl, carboxyl or a group equivalent to the carboxyl group, or a group, optionally substituted by at least one radical M², selected from the group consisting of amino, (C₁-C₄)-alkyl, (C₁-C₄)-haloalkyl, (C₁-C₄)-alkoxy, (C₁-C₄)-haloalkoxy, (C₁-C₄)-alkoxy-(C₁-C₄)-alkoxy, (C₁-C₄)-alkoxy-(C₁-C₄)-alkyl, (C₁-C₄)-alkylsulphanyl, (C₁-C₄)-haloalkylsulphanyl (C₁-C₄)-alkoxycarbonyl, (C₁-C₄)-alkylcarbonyl, carbamoyl, mono- and —N,N-di-(C₁-C₄)-alkylaminocarbonyl, (C₁-C₄)-acylamino, mono- and —N,N-di-(C₁-C₄)-alkylamino, tri-(C₁-C₄)-alkylsilyl, (C₃-C₆)-cycloalkyl, C₆-aryl, heterocyclyl having 3 to 6 ring atoms, where each of the last-mentioned cyclic groups may also be attached via heteroatoms or a divalent functional —CH₂— or —C₂H₄— group, (C₁-C₄)-alkylsulphinyl, where both enantiomers of the (C₁-C₄)-alkylsulphonyl group are included, (C₁-C₄)-alkylsulphonyl, (C₁-C₄)-alkylphosphinyl, (C₁-C₄)-alkylsulphanyl-(C₁-C₄)-alkyl, (C₁-C₄)-alkoxy-(C₁-C₄)-alkyl, mono- and N,N-di-(C₁-C₄)-alkylamino-(C₁-C₄)-alkyl and hydroxy-(C₁-C₄)-alkyl;
M² represents amino, hydroxy, halogen, nitro, cyano, isocyano, mercapto, isothiocyanato, carboxyl or carboxamide.

A preferred embodiment relates to compounds of the formula (I) in which
R¹ represents hydrogen, represents, optionally substituted by at least one radical M¹, C₁-C₆-alkyl, C₃-C₆-alkenyl, C₃-C₆-alkynyl, C₃-C₇-cycloalkyl, C₁-C₆-alkylcarbonyl, C₁-C₆-alkoxycarbonyl, aryl-(C₁-C₃)-alkyl, heteroaryl-(C₁-C₃)-alkyl;
W represents oxygen;
Q represents hydrogen, formyl, hydroxy, or one of the moieties, optionally substituted by at least one radical M¹, amino, C₁-C₆-alkyl, C₃-C₆-alkenyl, C₃-C₆-alkynyl, C₃-C₆-cycloalkyl, C₂-C₇-heterocycloalkyl, C₁-C₄-alkoxy, C₃-C₆-cycloalkyl-C₁-C₆-alkyl, aryl-(C₁-C₃)-alkyl, heteroaryl-(C₁-C₃)-alkyl, N—C₁-C₄-alkylamino, N—(C₁-C₄)-alkylcarbonylamino, N,N-di-(C₁-C₄)-alkylamino; or
Q represents an unsaturated 6-membered carbocycle which is optionally mono- or polysubstituted by V or represents an unsaturated 5- or 6-membered heterocyclic ring which is optionally mono- or polysubstituted by V, where
T represents one of the 5-membered heteroaromatics T1-T8 as defined in paragraph [58],
where
R⁶ in T1-T8 in formula (I) independently of one another represent halogen, cyano, nitro, an amino optionally substituted by at least one radical M¹, or a group consisting of (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl, each of which is optionally substituted by at least one halogen, where the group in question substituted by at least one halogen may optionally be substituted by at least one radical M¹;
n in T1-T8 represents the value 0 or 1;
Z¹ represents (C₁-C₆)-haloalkyl optionally substituted by at least one radical M¹;
Z² represents hydrogen, halogen, cyano, nitro or, optionally substituted by at least one radical M¹, amino, (C₁-C₆)-alkyl, (C₁-C₆)-alkylcarbonyl, (C₁-C₆)-alkylsulphanyl, (C₁-C₆)-alkylsulphinyl, (C₁-C₆)-alkylsulphonyl;
Z³ represents hydrogen or, optionally substituted by at least one radical M¹, (C₁-C₆)-alkyl, (C₃-C₆)-cycloalkyl, (C₃-C₆)-alkenyl, (C₃-C₆)-alkynyl, aryl or hetaryl;
   and all other parameters (such as, for example, A₁ to A₄, but where no more than three of the chemical moieties A₁ to A₄ simultaneously represent nitrogen) are as defined in paragraph [58].

A further preferred embodiment relates to compounds of the formula (I) in which
R¹ represents hydrogen, represents (C₁-C₆)-alkyl, (C₃-C₆)-alkenyl, (C₃-C₆)-alkynyl, (C₃-C₇)-cycloalkyl, (C₁-C₆)-alkylcarbonyl, (C₁-C₆)-alkoxycarbonyl, aryl-(C₁-C₃)-alkyl, heteroaryl-(C₁-C₃)-alkyl which are optionally mono- to heptasubstituted independently of one another by fluorine, chlorine, cyano, (C₁-C₆)-alkoxy and (C₁-C₆)-alkoxycarbonyl;
R², R³, R⁴ and R⁵ independently of one another represent hydrogen, fluorine, chlorine, bromine, iodine, cyano, nitro, or a (C₁-C₆)-alkyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino or N,N-di-(C₁-C₆)-alkylamino which are optionally mono- to pentasubstituted independently of one another by hydroxy, amino, nitro, fluorine, chlorine, cyano, (C₁-C₆)-alkoxy, carboxyl, (C₁-C₆)-alkoxycarbonyl, (C₁-C₆)-alkylcarbonyl or phenyl which is optionally substituted by at least one M²;
Q represents hydrogen, amino or one of the moieties (C₁-C₆)-alkyl, (C₃-C₆)-alkenyl, (C₃-C₆)-alkynyl, (C₃-C₆)-cycloalkyl, (C₂-C₅)-heterocycloalkyl, (C₁-C₄)-alkoxy, (C₃-C₆)-cycloalkyl-(C₁-C₆)-alkyl, aryl-(C₁-C₃)-alkyl, heteroaryl-(C₁-C₃)-alkyl, N—(C₁-C₄)-alkylamino, N—(C₁-C₄)-alkylcarbonylamino or N,N-di-(C₁-C₄)-alkylamino which are optionally independently of one another mono- to pentasubstituted by hydroxy, nitro, amino, fluorine, chlorine, (C₁-C₆)-alkyl, (C₁-C₆)-alkoxy, cyano, hydroxycarbonyl, (C₁-C₄)-alkoxycarbonyl, (C₁-C₄)-alkylcarbamoyl, (C₃-C₇)-cycloalkylcarbamoyl, optionally substituted by at least one phenyl which for its turn may optionally be substituted by at least one M²; or
Q represents 6-membered aryl substituted by 0-4 substituents V or a 5- or 6-membered heteroaromatic substituted by 0-4 substituents V, where
V independently of one another represent halogen, cyano, nitro or, optionally substituted by at least one radical M¹, (C₁-C₄)-alkyl, (C₂-C₄)-alkenyl, (C₂-C₄)-alkynyl, (C₃-C₆)-cycloalkyl, (C₁-C₄)-alkoxy, N—(C₁-C₄)-alkoxyimino-(C₁-C₃)-alkyl, (C₁-C₄)-alkylsulphanyl, (C₁-C₄)-alkylsulphinyl, (C₁-C₄)-alkylsulphonyl, N,N-di-((C₁-C₄)-alkyl)amino;
V independently of one another represent halogen, cyano, nitro or, optionally substituted by at least one radical M¹, (C₁-C₄)-alkyl, (C₂-C₄)-alkenyl, (C₂-C₄)-alkynyl, (C₃-C₆)-cycloalkyl, (C₁-C₄)-alkoxy, N—(C₁-C₄)-alkoxyimino-(C₁-C₃)-alkyl, (C₁-C₄)-alkylsulphanyl, (C₁-C₄)-alkylsulphinyl, (C₁-C₄)-alkylsulphonyl, N,N-di-((C₁-C₄)-alkyl)amino;
T represents one of the listed 5-membered heteroaromatics T1-T8 as defined in paragraph [58], where
R⁶ in T1-T8 independently of one another represent fluorine, chlorine, bromine, iodine, cyano, nitro, amino or represents (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl which are optionally independently of one another mono- to pentasubstituted by fluorine and/or chlorine;
n in T1-T8 represents the value 0 or 1;
Z¹ represents (C₁-C₆)-haloalkyl optionally substituted by at least one radical M¹;
Z² represents hydrogen, fluorine, chlorine, bromine, iodine, cyano, nitro, amino or represents (C₁-C₆)-alkyl, (C₁-C₆)-alkylcarbonyl, (C₁-C₆)-alkylsulphanyl, (C₁-C₆)-alkylsulphinyl, (C₁-C₆)-alkylsulphonyl which are optionally independently of one another mono- to pentasubstituted by fluorine and/or chlorine;
Z³ represents hydrogen or optionally substituted (C₁-C₆)-alkyl, (C₃-C₆)-cycloalkyl, (C₃-C₄)-alkenyl, (C₃-C₄)-alkynyl, 6-membered aryl or 5- or 6-membered hetaryl;
   all other parameters (such as, for example, A₁ to A₄, but where no more than three of the chemical moieties A₁ to A₄ simultaneously represent nitrogen) are as defined in paragraph [58].

A further preferred embodiment relates to compounds of the formula (I)

in which
R¹ represents hydrogen or represents (C₁-C₄)-alkyl, (C₂-C₄)-alkenyl, (C₂-C₄)-alkynyl, (C₃-C₆)-cycloalkyl, (C₁-C₄)-alkylcarbonyl, (C₁-C₄)-alkoxycarbonyl, aryl-(C₁-C₂)-alkyl, heteroaryl-(C₁-C₂)-alkyl which are optionally mono- to pentasubstituted independently of one another by fluorine, chlorine, cyano, C₁-C₄-alkoxy and C₁-C₄-alkoxycarbonyl;
R², R³, R⁴ and R⁵ independently of one another represent hydrogen, fluorine, chlorine, bromine, iodine, cyano, nitro or represent (C₁-C₄)-alkyl, C₃-C₆-cycloalkyl, (C₁-C₄)-alkoxy, N—(C₁-C₄)-alkoxyimino-C₁-C₂-alkyl, (C₁-C₄)-alkylsulphanyl, (C₁-C₄)-alkylsulphinyl, (C₁-C₄)-alkylsulphonyl, N—(C₁-C₄)-alkylamino or N,N-di-(C₁-C₄)-alkylamino which are optionally mono- to pentasubstituted independently of one another by hydroxy, nitro, amino, fluorine, chlorine, (C₁-C₄)-alkoxy, cyano, hydroxycarbonyl, (C₁-C₄)-alkylcarbonyl, (C₁-C₄)-alkoxycarbonyl or phenyl;
Q represents hydrogen, amino or represents one of the moieties (C₁-C₄)-alkyl, (C₂-C₆)-alkenyl, (C₂-C₆)-alkynyl, (C₃-C₆)-cycloalkyl, C₂-C₅-heterocycloalkyl, (C₁-C₄)-alkoxy, (C₃-C₆)-cycloalkyl-(C₁-C₆)-alkyl, aryl-(C₁-C₃)-alkyl, heteroaryl-(C₁-C₃)-alkyl, N—(C₁-C₄)-alkylamino, N—(C₁-C₄)-alkylcarbonylamino or N,N-di-(C₁-C₄)-alkylamino which are optionally independently of one another mono- to pentasubstituted by hydroxy, nitro, amino, halogen, (C₁-C₄)-alkyl, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl; or
Q represents aryl substituted by 0, 1, 2, 3 or 4 substituents V or a 5- or 6-membered heteroaromatic substituted by 0, 1, 2, 3 or 4 substituents V, where
V independently of one another represent fluorine, chlorine, bromine, iodine, cyano, nitro, represents (C₁-C₄)-alkyl, (C₂-C₄)-alkenyl, (C₂-C₄)-alkynyl, (C₃-C₆)-cycloalkyl, (C₁-C₄)-alkoxy, N—(C₁-C₄)-alkoxyimino-(C₁-C₃)-alkyl, (C₁-C₄)-alkylsulphanyl, (C₁-C₄)-alkylsulphinyl, (C₁-C₄)-alkylsulphonyl, N,N-di-((C₁-C₄)-alkyl)amino which are optionally independently of one another mono- to pentasubstituted by hydroxy, nitro, amino, fluorine, chlorine, bromine, iodine, (C₁-C₄)-alkyl, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl;
T represents a 5-membered heteroaromatic T1-T8 as defined in paragraph [58],
where
R⁶ in T1-T8 independently of one another represent fluorine, chlorine, cyano, nitro, amino or represents (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl which are optionally mono- to pentasubstituted by fluorine and/or chlorine, and
n in T1-T8 represents the value 0 or 1;
Z¹ represents (C₁-C₄)-haloalkyl which is optionally independently of one another mono- or disubstituted by (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl,
Z² represents hydrogen, fluorine, chlorine, bromine, iodine, cyano, nitro, amino or represents (C₁-C₄)-alkyl, (C₁-C₄)-alkylcarbonyl, (C₁-C₄)-alkylsulphanyl, (C₁-C₄)-alkylsulphinyl, (C₁-C₄)-alkylsulphonyl which are optionally independently of one another mono- to trisubstituted by hydroxy, nitro, amino, fluorine, chlorine, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl, and
Z³ represents hydrogen or represents (C₁-C₄)-alkyl, (C₃-C₆)-cycloalkyl, (C₂-C₄)-alkenyl, (C₂-C₄)-alkynyl, phenyl and hetaryl which are optionally independently of one another mono- to trisubstituted by hydroxy, nitro, amino, (C₁-C₄)-alkoxy, cyano, fluorine, chlorine, hydroxycarbonyl, (C₁-C₄)-alkoxycarbonyl, (C₁-C₄)-alkylcarbamoyl, (C₃-C₆)-cycloalkylcarbamoyl, phenyl;
   and all other parameters (such as, for example, A₁ to A₄, but where no more than three of the chemical moieties A₁ to A₄ simultaneously represent nitrogen) are as defined in paragraph [58].

Particular preference is furthermore given to compounds of the formula (I) in which
R¹ represents hydrogen, methyl, ethyl, n-propyl, isopropyl, n-butyl, isobutyl, s-butyl, t-butyl, methoxymethyl, ethoxymethyl, propoxymethyl, methylcarbonyl, ethylcarbonyl, n-propylcarbonyl, isopropylcarbonyl, n-butylcarbonyl, isobutylcarbonyl, s-butylcarbonyl, t-butylcarbonyl, methoxycarbonyl, ethoxycarbonyl, n-propoxycarbonyl, isopropoxycarbonyl, n-butoxycarbonyl, isobutoxycarbonyl, s-butoxycarbonyl, t-butoxycarbonyl, cyanomethyl, 2-cyanoethyl, benzyl, 4-methoxybenzyl, pyrid-2-ylmethyl, pyrid-3-ylmethyl, pyrid-4-ylmethyl, 6-chloropyrid-3-ylmethyl;
the chemical moieties
A₁ represents CR² or nitrogen,
A₂ represents CR³ or nitrogen,
A₃ represents CR⁴ or nitrogen, and
A₄ represents CR⁵ or nitrogen,
but where not more than three of the chemical moieties A₁ to A₄ simultaneously represent nitrogen;
R² and R⁵ independently of one another represent hydrogen, methyl, fluorine or chlorine; and
R³ and R⁴ are each independently hydrogen, fluorine, chlorine, bromine, iodine, cyano, nitro, methyl, ethyl, fluoromethyl, difluoromethyl, chlorodifluoromethyl, trifluoromethyl, 2,2,2-trifluoroethyl, methoxy, ethoxy, n-propoxy, 1-methylethoxy, fluoromethoxy, difluoromethoxy, chlorodifluoromethoxy, dichlorofluoromethoxy, trifluoromethoxy, 2,2,2-trifluoroethoxy, 2-chloro-2,2-difluoroethoxy, pentafluoroethoxy, N-methoxyiminomethyl, 1-(N-methoxyimino)ethyl, methylsulphanyl, trifluoromethylsulphanyl, methylsulphonyl, methylsulphinyl, trifluoromethylsulphonyl, trifluoromethylsulphinyl;
W represents oxygen or sulphur;
Q represents hydrogen, methyl, ethyl, n-propyl, 1-methylethyl, 1,1-dimethylethyl, 1-methylpropyl, n-butyl, 2-methylpropyl, 2-methylbutyl, hydroxyethyl, 2-hydroxypropyl, cyanomethyl, 2-cyanoethyl, 2-fluoroethyl, 2,2-difluoroethyl, 2,2,2-trifluoroethyl, 1-trifluoromethylethyl, 2,2-difluoropropyl, 3,3,3-trifluoropropyl, 2,2-dimethyl-3-fluoropropyl, cyclopropyl, 1-methylcyclopropyl, 1-cyanocyclopropyl, 1-methoxycarbonylcyclopropyl, 1-(N-methylcarbamoyl)cyclopropyl, 1-carbamoylcyclopropyl, 1-carbamothioylcyclopropyl, 1-(N-cyclopropylcarbamoyl)cyclopropyl, cyclopropylmethyl, cyclobutyl, cyclopentyl, cyclohexyl, 1-cyclopropylethyl, bis(cyclopropyl)methyl, 2,2-dimethylcyclopropylmethyl, 2-phenylcyclopropyl, 2,2-dichlorocyclopropyl, trans-2-chlorocyclopropyl, cis-2-chlorocyclopropyl, 2,2-difluorocyclopropyl, trans-2-fluorocyclopropyl, cis-2-fluorocyclopropyl, trans-4-hydroxycyclohexyl, 4-trifluoromethylcyclohexyl, prop-2-enyl, 2-methylprop-2-enyl, prop-2-ynyl, 1,1-dimethylbut-2-ynyl, 3-chloroprop-2-enyl, 3-fluoroprop-2-enyl, 3,3-dichloroprop-2-enyl, 3,3-dichloro-1,1-dimethylprop-2-enyl, oxetan-3-yl, thietan-3-yl, 1-oxidothietan-3-yl, 1,1-dioxidothietan-3-yl, isoxazol-3-ylmethyl, 1,2,4-triazol-3-ylmethyl, 3-methyloxetan-3-ylmethyl, benzyl, 2,6-difluorophenylmethyl, 3-fluorophenylmethyl, 2-fluorophenylmethyl, 2,5-difluorophenylmethyl, 1-phenylethyl, 4-chlorophenylethyl, 2-trifluoromethylphenylethyl, 1-pyridin-2-ylethyl, pyridin-2-ylmethyl, 5-fluoropyridin-2-ylmethyl, (6-chloropyridin-3-yl)methyl, pyrimidin-2-ylmethyl, methoxy, 2-ethoxyethyl, 2-methoxyethyl, 2-(methylsulphanyl)ethyl, 1-methyl-2-(ethylsulphanyl)ethyl, 2-methyl-1-(methylsulphanyl)propan-2-yl, methoxycarbonyl, methoxycarbonylmethyl, NH₂, N-ethylamino, N-allylamino, N,N-dimethylamino, N,N-diethylamino; or
Q represents phenyl, pyridazine, pyrazine, pyrimidine, triazine, pyridine, pyrazole, thiazole, isothiazole, oxazole, isoxazole, triazole, imidazole, furan, thiophene, pyrrole, oxadiazole, thiadiazole each substituted by 0, 1, 2 or 3 substituents V, where
V independently of one another represent fluorine, chlorine, bromine, iodine, cyano, nitro, methyl, ethyl, difluoromethyl, trichloromethyl, chlorodifluoromethyl, dichlorofluoromethyl, trifluoromethyl, 1-fluoroethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, heptafluoro-n-propyl, heptafluoroisopropyl, nonafluoro-n-butyl, cyclopropyl, cyclobutyl, methoxy, ethoxy, n-propoxy, 1-methylethoxy, fluoromethoxy, difluoromethoxy, chlorodifluoromethoxy, dichlorofluoromethoxy, trifluoromethoxy, 2,2,2-trifluoroethoxy, 2-chloro-2,2-difluoroethoxy, pentafluoroethoxy, N-methoxyiminomethyl, 1-(N-methoxyimino)ethyl, methylsulphanyl, methylsulphonyl, methylsulphinyl, trifluoromethylsulphonyl, trifluoromethylsulphinyl, trifluoromethylsulphanyl, —N,N-dimethylamino;
T represents one of the 5-membered heteroaromatics T1-T8 listed below, as defined above, where
R⁶ in T1-T8 independently of one another represent fluorine, chlorine, cyano, nitro, amino, methyl, ethyl, propyl, 1-methylethyl, tert-butyl, trifluoromethyl, difluoromethyl, methoxy, ethoxy, trifluoromethoxy, 2,2-difluoroethoxy, 2,2,2-trifluoroethoxy, methylcarbonyl, ethylcarbonyl, trifluoromethylcarbonyl, methylsulphanyl, methylsulphinyl, methylsulphonyl, trifluoromethylsulphonyl, trifluoromethylsulphanyl, trifluoromethylsulphinyl, and
n in T1-T8 represents the value 0 or 1;
Z¹ represents difluoromethyl, trichloromethyl, chlorodifluoromethyl, dichlorofluoromethyl, trifluoromethyl, bromodichloromethyl, 1-fluoroethyl, 1-fluoro-1-methylethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, heptafluoro-n-propyl, heptafluoroisopropyl, nonafluoro-n-butyl and
Z² represents hydrogen, fluorine, chlorine, bromine, iodine, cyano, nitro, amino, methyl, ethyl, 1,1-t-butyl, difluoromethyl, trichloromethyl, chlorodifluoromethyl, dichlorofluoromethyl, trifluoromethyl, bromodichloromethyl, 1-fluoroethyl, 1-fluoro-1-methylethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, heptafluoro-n-propyl, heptafluoroisopropyl, nonafluoro-n-butyl, methylsulphanyl, methylsulphinyl, methylsulphonyl, ethylthio, ethylsulphinyl, ethylsulphonyl, trifluoromethylsulphanyl, trifluoromethylsulphinyl, trifluoromethylsulphonyl, chlorodifluoromethylsulphanyl, chlorodifluoromethylsulphinyl, chlorodifluoromethylsulphonyl, dichlorofluoromethylsulphanyl, dichlorofluoromethylsulphinyl, dichlorofluoromethylsulphonyl and
Z³ represents hydrogen, methyl, ethyl, n-propyl, isopropyl, n-butyl, isobutyl, s-butyl, t-butyl, 1-propenyl, 1-propynyl, 1-butynyl, difluoromethyl, trichloromethyl, chlorodifluoromethyl, dichlorofluoromethyl, trifluoromethyl, 1-fluoroethyl, 1-fluoro-1-methylethyl, 2-fluoroethyl, 2,2-difluoroethyl, 2,2,2-trifluoroethyl, phenyl, 2-chlorophenyl, 3-chlorophenyl, 4-chlorophenyl, 2,4-dichlorophenyl, 2,5-dichlorophenyl, 3,4-dichlorophenyl, 2,6-dichlorophenyl, 2,6-dichloro-4-trifluoromethylphenyl, 3-chloro-5-trifluoromethylpyridin-2-yl.

Very especially preferred compounds for the purpose of the invention are those of the general formula (I) in which
Z¹ represents trifluoromethyl or pentafluoroethyl;
Z² represents trifluoromethyl, nitro, methylsulphanyl, methylsulphinyl, methylsulphonyl, fluorine, chlorine, bromine, cyano or iodine;
Z³ represents methyl, ethyl, n-propyl or hydrogen;
R¹ represents hydrogen, methyl, ethyl, n-propyl, isopropyl, n-butyl, isobutyl, s-butyl, t-butyl, methoxymethyl, ethoxymethyl, propoxymethyl, methylcarbonyl, ethylcarbonyl, n-propylcarbonyl, isopropylcarbonyl, n-butylcarbonyl, isobutylcarbonyl, s-butylcarbonyl, t-butylcarbonyl, methoxycarbonyl, ethoxycarbonyl, n-propoxycarbonyl, isopropoxycarbonyl, n-butoxycarbonyl, isobutoxycarbonyl, s-butoxycarbonyl, t-butoxycarbonyl, cyanomethyl, 2-cyanoethyl, benzyl, 4-methoxybenzyl, pyrid-2-ylmethyl, pyrid-3-ylmethyl, pyrid-4-ylmethyl, 6-chloropyrid-3-ylmethyl;
A₁ and A₄ represent CH;
A₂ represents CR³ or N;
A₃ represents CR⁴; and
R³ and R⁴ represent fluorine, chlorine, bromine, iodine, methyl, ethyl, methylsulphanyl, methylsulphinyl or methylsulphonyl;
T represents one of the 5-membered heteroaromatics T1-T8 as defined above;
where
R⁶ in T1-T8 represents hydrogen, methyl, ethyl, 2-methylethyl, 2,2-dimethylethyl, fluorine, chlorine, bromine, iodine, nitro, trifluoromethyl, amino;
n in T1-T8 represents the value 0 or 1; preferably represents 0;
W represents oxygen; and
Q represents hydrogen, methyl, ethyl, n-propyl, 1-methylethyl, 1,1-dimethylethyl, n-butyl, 1-methylpropyl, 2-methylpropyl, 2-methylbutyl, hydroxyethyl, 2-hydroxypropyl, cyanomethyl, 2-cyanoethyl, 2-fluoroethyl, 2,2-difluoroethyl, 2,2,2-trifluoroethyl, 1-trifluoromethylethyl, 2,2-difluoropropyl, 3,3,3-trifluoropropyl, 2,2-dimethyl-3-fluoropropyl, cyclopropyl, 1-cyanocyclopropyl, 1-methoxycarbonylcyclopropyl, 1-(N-methylcarbamoyl)cyclopropyl, 1-carbamoylcyclopropyl, 1-carbamothioylcyclopropyl, 1-(N-cyclopropylcarbamoyl)cyclopropyl, cyclopropylmethyl, cyclobutyl, cyclopentyl, cyclohexyl, 1-cyclopropylethyl, bis(cyclopropyl)methyl, 2,2-dimethylcyclopropylmethyl, 2-phenylcyclopropyl, 2,2-dichlorocyclopropyl, trans-2-chlorocyclopropyl, cis-2-chlorocyclopropyl, 2,2-difluorocyclopropyl, trans-2-fluorocyclopropyl, cis-2-fluorocyclopropyl, trans-4-hydroxycyclohexyl, 4-trifluoromethylcyclohexyl, prop-2-enyl, 2-methylprop-2-enyl, prop-2-ynyl, 1,1-dimethylbut-2-ynyl, 3-chloroprop-2-enyl, 3-fluoroprop-2-enyl, 3,3-dichloroprop-2-enyl, 3,3-dichloro-1,1-dimethylprop-2-enyl, oxetan-3-yl, thietan-3-yl, 1-oxidothietan-3-yl, 1,1-dioxidothietan-3-yl, isoxazol-3-ylmethyl, 1,2,4-triazol-3-ylmethyl, 3-methyloxetan-3-ylmethyl, benzyl, 2,6-difluorophenylmethyl, 3-fluorophenylmethyl, 2-fluorophenylmethyl, 2,5-difluorophenylmethyl, 1-phenylethyl, 4-chlorophenylethyl, 2-trifluoromethylphenylethyl, 1-pyridin-2-ylethyl, pyridin-2-ylmethyl, (6-chloropyridin-3-yl)methyl, 5-fluoropyridin-2-ylmethyl, pyrimidin-2-ylmethyl, methoxy, 2-ethoxyethyl, 2-methoxyethyl, 2-(methylsulphanyl)ethyl, 1-methyl-2-(ethylsulphanyl)ethyl, 2-methyl-1-(methylsulphanyl)propan-2-yl, methoxycarbonyl, methoxycarbonylmethyl, NH₂, N-ethylamino, N-allylamino, N,N-dimethylamino, N,N-diethylamino; or
Q represents phenyl, naphthyl, pyridazine, pyrazine, pyrimidine, triazine, pyridine, pyrazole, thiazole, isothiazole, oxazole, isoxazole, triazole, imidazole, furan, thiophene, pyrrole, oxadiazole, thiadiazole substituted by 0, 1, 2 or 3 substituents V; where
V independently of one another represent fluorine, chlorine, bromine, iodine, cyano, nitro, methyl, ethyl, difluoromethyl, trichloromethyl, chlorodifluoromethyl, dichlorofluoromethyl, trifluoromethyl, 1-fluoroethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, heptafluoro-n-propyl, heptafluoroisopropyl, nonafluoro-n-butyl, cyclopropyl, cyclobutyl, methoxy, ethoxy, n-propoxy, 1-methylethoxy, fluoromethoxy, difluoromethoxy, chlorodifluoromethoxy, dichlorofluoromethoxy, trifluoromethoxy, 2,2,2-trifluoroethoxy, 2-chloro-2,2-difluoroethoxy, pentafluoroethoxy, N-methoxyiminomethyl, 1-(N-methoxyimino)ethyl, methylsulphanyl, methylsulphonyl, methylsulphinyl, trifluoromethylsulphonyl, trifluoromethylsulphinyl, trifluoromethylsulphanyl, —N,N-dimethylamino.

Especially emphasized compounds for the purpose of the invention are those of the general formulae (Ia), (Ib), (Ic), (Id), (Ie), (If), (Ig), (Ih) in which the radicals A₁-A₄, n, W, Q, R¹, R⁶ and Z¹-Z³ have the meanings given above.

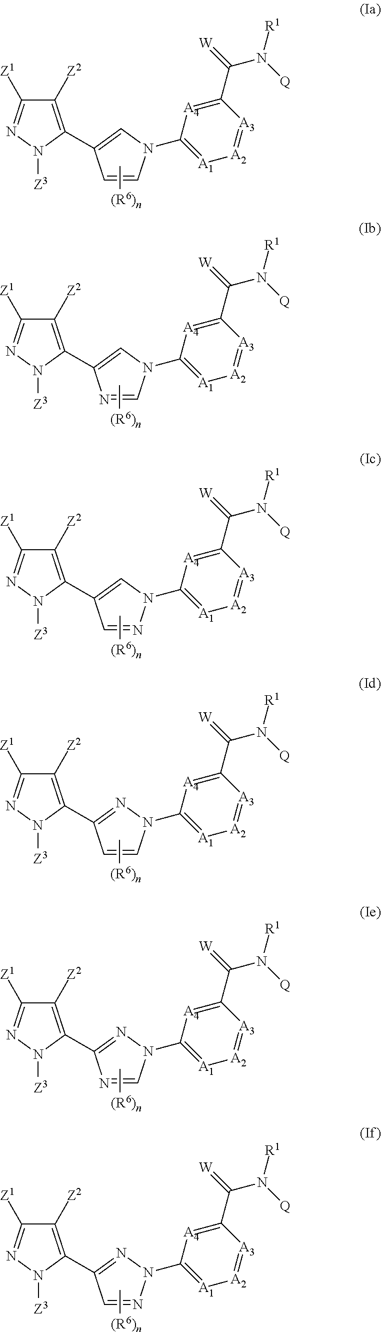

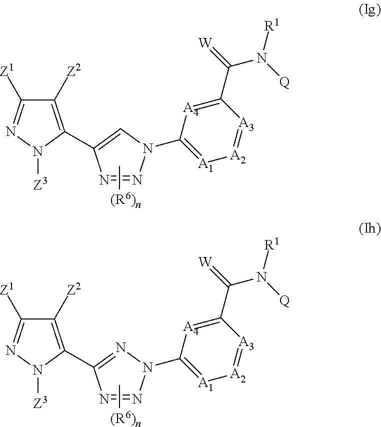

A preferred embodiment relates to compounds of the formulae (Ic), (If), (Ig) and (Ih), in other words to compounds of the formula (I) in which T represents T3 (formula (Ic)), T6 (formula (If), T7 (formula (Ig)) or T8 (formula (Ih)).

A further preferred embodiment relates to compounds of the formula (I) in which T represents T1 (formula (Ia)).

A further preferred embodiment relates to compounds of the formula (I) in which T represents T2 (formula (Ib)).

A further preferred embodiment relates to compounds of the formula (I) in which T represents T3 (formula (Ic)).

A further preferred embodiment relates to compounds of the formula (I) in which T represents T4 (formula (Id)).

A further preferred embodiment relates to compounds of the formula (I) in which T represents T5 (formula (Ie)).

A further preferred embodiment relates to compounds of the formula (I) in which T represents T6 (formula (If)).

A further preferred embodiment relates to compounds of the formula (I) in which T represents T7 (formula (Ig)).

A further preferred embodiment relates to compounds of the formula (I) in which T represents T8 (formula (Ih)).

A further preferred embodiment relates to compounds of the formula (I), preferably of the formulae (Ic), (If), (Ig) and (Ih), in which

A₁ represents CR², A₂ represents CR³, A₃ represents CR⁴ and A₄ represents CR⁵, where R², R³, R⁴ and R⁵ independently of one another represent hydrogen, halogen, cyano, nitro or optionally substituted (C₁-C₆)-alkyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino or N,N-di-(C₁-C₆)-alkylamino, preferably in which R², R³, R⁴ and R⁵ independently of one another represent hydrogen, halogen, cyano, nitro, (C₁-C₆)-alkyl, (C₁-C₆)-haloalkyl, (C₁-C₆)-alkoxy or (C₁-C₆)-haloalkoxy, particularly preferably in which R², R³, R⁴ and R⁵ independently of one another represent hydrogen, fluorine, chlorine, iodine, bromine, (C₁-C₄)-alkyl or (C₁-C₄)-alkoxy.

A further preferred embodiment relates to compounds of the formula (I), preferably of the formulae (Ic), (If), (Ig) and (Ih), in which

Z¹, Z² and Z³ independently of one another represent optionally fluorine- or chlorine-substituted (C₁-C₆)-alkyl; preference is given here for Z¹ and Z² to represent fluorine- or chlorine-substituted (C₁-C₆)-alkyl and for Z³ to represent (C₁-C₆)-alkyl; particular preference is given here for Z¹ and Z² to represent perfluorinated (C₁-C₄)-alkyl and for Z³ to represent (C₁-C₄)-alkyl; very particular preference is given here for Z¹ and Z² to represent perfluorinated ethyl (C₂F₅), for Z² to represent perfluorinated methyl (CF₃) and for Z³ to represent methyl.

A further preferred embodiment relates to compounds of the formula (I), preferably of the formulae (Ic), (If), (Ig) and (Ih), in which W represents oxygen.

A further preferred embodiment relates to compounds of the formula (I), preferably compounds of the formulae (Ic), (If), (Ig) and (Ih), in which Q represents hydrogen or an optionally cyano- or halogen-substituted group selected from the group consisting of (C₁-C₆)-alkyl, (C₃-C₆)-cycloalkyl, (C₁-C₆)-alkoxy.

A further preferred embodiment relates to compounds of the formula (I), preferably of the formulae (Ic), (If), (Ig) and (Ih), in which

n in T represents 0 or 1 and R⁶ represents fluorine, chlorine, bromine, iodine, (C₁-C₄)-alkyl, (C₁-C₄)-haloalkyl, (C₁-C₄)-alkoxy, (C₁-C₄)-haloalkoxy, cyano, nitro or amino.

A further preferred embodiment relates to compounds of the formula (I), preferably of the formulae (Ic), (If), (Ig) and (Ih), in which R¹ represents hydrogen, (C₁-C₄)-alkyl or (C₁-C₄)-haloalkyl.

A further preferred embodiment relates to compounds of the formula (I), preferably of the formulae (Ic), (If), (Ig) and (Ih), in which

A₁ represents CR², A₂ represents CR³, A₃ represents CR⁴ and A₄ represents CR⁵ and where R², R³, R⁴ and R⁵ independently of one another represent hydrogen, halogen, cyano, nitro, (C₁-C₆)-alkyl, (C₁-C₆)-haloalkyl, (C₁-C₆)-alkoxy or (C₁-C₆)-haloalkoxy, particularly preferably hydrogen, fluorine, chlorine, iodine, bromine, (C₁-C₄)-alkyl or (C₁-C₄)-alkoxy,
Z¹ and Z² independently of one another represent perfluorinated (C₁-C₄)-alkyl and Z³ represents (C₁-C₄)-alkyl; here, very particularly preferably Z¹ represents perfluorinated ethyl (C₂F₅), Z² represents perfluorinated methyl (CF₃) and Z³ represents methyl.
W represents oxygen
n in T represents 0 or 1 and R⁶ represents fluorine, chlorine, bromine, iodine, (C₁-C₄)-alkyl, (C₁-C₄)-haloalkyl, (C₁-C₄)-alkoxy, (C₁-C₄)-haloalkoxy, cyano, nitro or amino
R¹ represents hydrogen, (C₁-C₄)-alkyl or (C₁-C₄)-haloalkyl.

A further preferred embodiment relates to compounds of the formula (I), selected from the formulae (Ic), (If), (Ig) and (Ih),

in which
A₁ represents CR², A₂ represents CR³, A₃ represents CR⁴ and A₄ represents CR⁵ and where
R², R³, R⁴ and R⁵ independently of one another represent hydrogen, fluorine, chlorine, iodine, bromine or (C₁-C₄)-alkyl,
Z¹ and Z² independently of one another represent perfluorinated (C₁-C₄)-alkyl and Z³ represents (C₁-C₄)-alkyl; here, very particularly preferably Z¹ represents perfluorinated ethyl (C₂F₅), Z² represents perfluorinated methyl (CF₃) and Z³ represents methyl.
W represents oxygen
n in T represents 0 or 1 and R⁶ represents fluorine, chlorine, bromine, iodine, halogen, (C₁-C₄)-alkyl, (C₁-C₄)-haloalkyl, (C₁-C₄)-alkoxy, (C₁-C₄)-haloalkoxy, cyano, nitro or amino
R¹ represents hydrogen, (C₁-C₄)-alkyl or (C₁-C₄)-haloalkyl.

### Process

Reaction Scheme 1 shows the general Preparation Process A for the compounds (I-c) according to the invention.

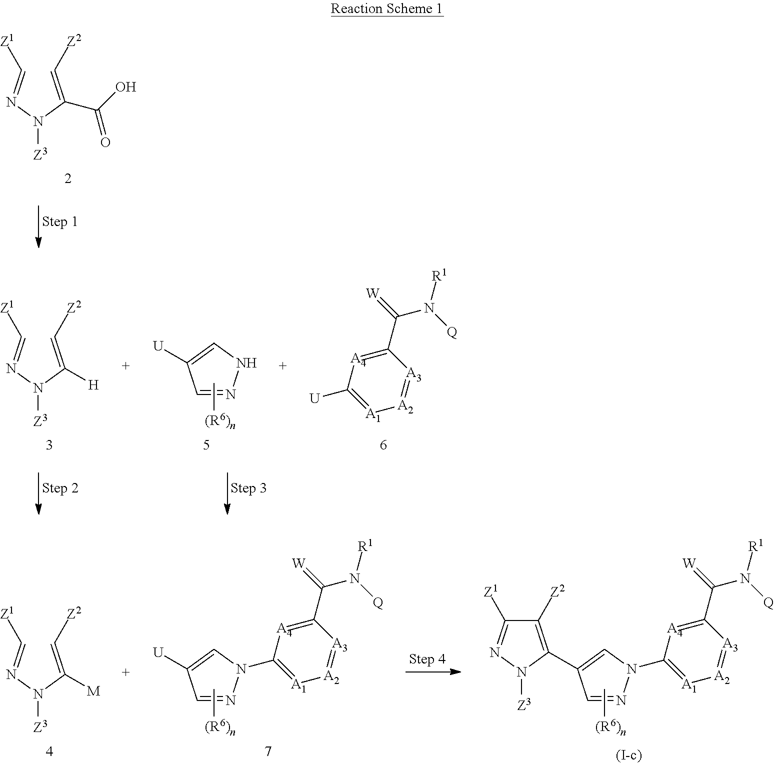

The radicals Z¹-Z³, R¹, R⁶, n, A₁-A₄, Q and W have the meanings described above. U is bromine, iodine or triflate when M is a boronic acid, boronic ester or trifluoroboronate. U is a boronic acid, boronic ester or trifluoroboronate when M is bromine, iodine or triflate.

Compounds according to the invention of the general structure (I-c) can be prepared by processes known from the literature by reacting intermediate 4 with derivatives of the general structure 7 [e.g. M=B(OR)₂, U═Br, Suzuki reaction, palladium catalysis, analogous reactions are described here: WO2005-040110; WO2009-089508]. The intermediates of the general structure 4 can, for example for M=B(OR)₂, be prepared by deprotonation of the 5H-pyrazoles of type 3 with lithium diisopropylamide (LDA) and subsequent reaction with boron derivatives of the B(OR)₃ type to give boric esters (see the description of analogous reactions in Org. Biomol. Chem. 2009, 7, 2155-2161). The 5H-pyrazoles 3 can be prepared, for example, by decarboxylation of the pyrazole-5-carboxylic acid 2 (see, for example, Can. J. Chem. 1986, 64, 11, 2211-2219; Eur. J. Org. Chem. 2013, 30, 6811-6822). The compounds of the general structures 5 and 6 are either commercially available or can be prepared by processes known to the person skilled in the art. The intermediates 7 are prepared, for example, by copper-catalysed reaction of 6 (U═Br) with the NH-pyrazole 5 according to processes known from the literature (e.g. Tetrahedron 2011, 67, 5282-5288; EP2221298; US2008/153852).

Reaction Scheme 2 shows the general Preparation Process B for the compounds (I-g) according to the invention.

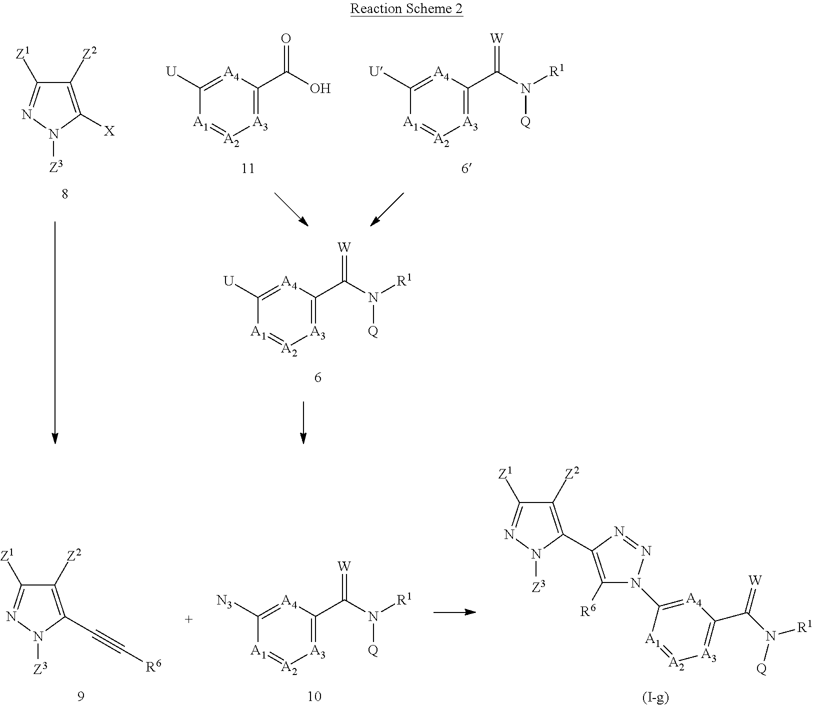

The radicals A₁-A₄, R¹, R⁶, Q, W and Z¹-Z³ have the meanings described above. X represents a leaving group such as, for example, a halogen, e.g. fluorine. U and U′ represent bromine, iodine or triflate. Compounds according to the invention of the general structure (I-g) can be prepared by processes known from the literature by reacting alkynes of the general structure 9 with azides of the general structure 10 (see, for example, Tetrahedron 2012, 68, 7861-7866; J. Am. Chem. Soc. 2008, 130, 12111-12122). Azides of the general structure 10 can be prepared, for example, from halides of the general structure 6 (see, for example, Org. Biomol. Chem. 2013, 11, 938). Compounds of the general structure 6 can be prepared, for example, by amide formation from the corresponding acids 11 or else by halogen exchange from corresponding intermediates of the general formula 6′ (see, for example, J. Am. Chem. Soc. 2002, 124, 14844-14845). Compounds of the general structures 11 and 6′ are either commercially available or can be prepared by processes known to the person skilled in the art. The compounds of the general structure 9 can be prepared by processes known from the literature, for example by nucleophilic substitution at the heteroaromatic (X=chlorine or fluorine) (WO2007-107470; Tetrahedron Letters 2003, 44, 7629-7632) or by a transition metal-catalysed reaction (X=bromine or iodine) (WO2012-003405; WO2009-158371) from the appropriate starting materials 8.

Reaction Scheme 3 shows the general Preparation Process B for the compounds (I-f) according to the invention.

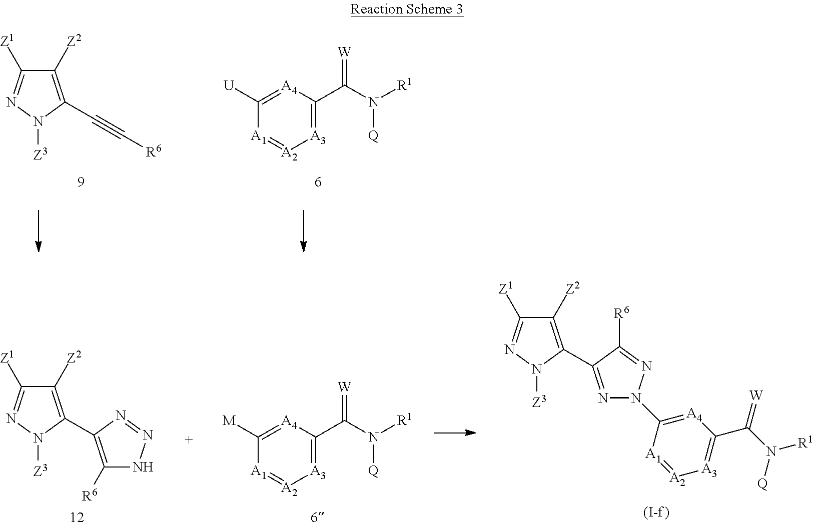

The radicals A₁-A₄, R¹, R⁶, Q, W and Z¹-Z³ have the meanings described above. U represents bromine, iodine or triflate, whereas M represents a boronic acid, boronic ester or trifluoroboronate. Compounds according to the invention of the general structure (I-f) can be prepared by processes known from the literature by reacting 1,2,3-triazoles of the general structure 12 with boron compounds of the general structure 6″ [see, for example, Org. Lett. 2008, 10, 5389-5392; Bioorg. Med. Chem. Lett. 2003, 13, 1665-1668]. Triazoles of the general structure 12 can be prepared, for example, from alkynes of the general structure 9 by reaction with azides [see, for example, Org. Lett. 2008, 10, 3171-3174]. Boron compounds of the general structure 6″ can be prepared, for example, from the corresponding halogen compounds 6 [see, for example, WO2006/080884; WO2006/025783].

Reaction Scheme 4 shows the general Preparation Process B for the compounds (I-h) according to the invention.

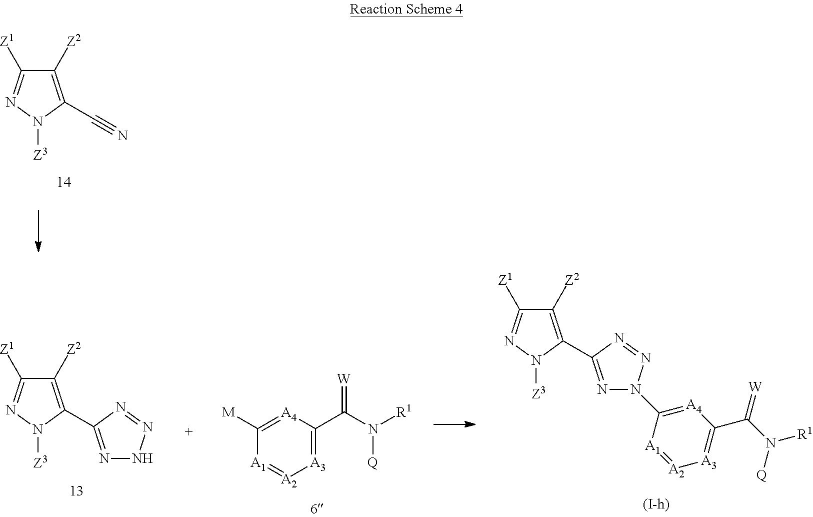

The radicals A₁-A₄, R¹, Q, W and Z¹-Z³ have the meanings described above. M represents a boronic acid, boronic ester or trifluoroboronate. Compounds according to the invention of the general structure (I-h) can be prepared by processes known from the literature by reacting tetrazoles of the general structure 13 with boron compounds of the general structure 6″ [see, for example, Tetrahedron Lett. 1998, 39, 2941-2944; Chem. Commun. 2012, 48, 2719-2721]. Tetrazoles of the general structure 13 can be prepared, for example, from nitriles of the general structure 14 by reaction with azides [see, for example, US2007/23876; WO2014/2109].

Reaction Scheme 5 shows the general Preparation Process B for the compounds (I-d) according to the invention.

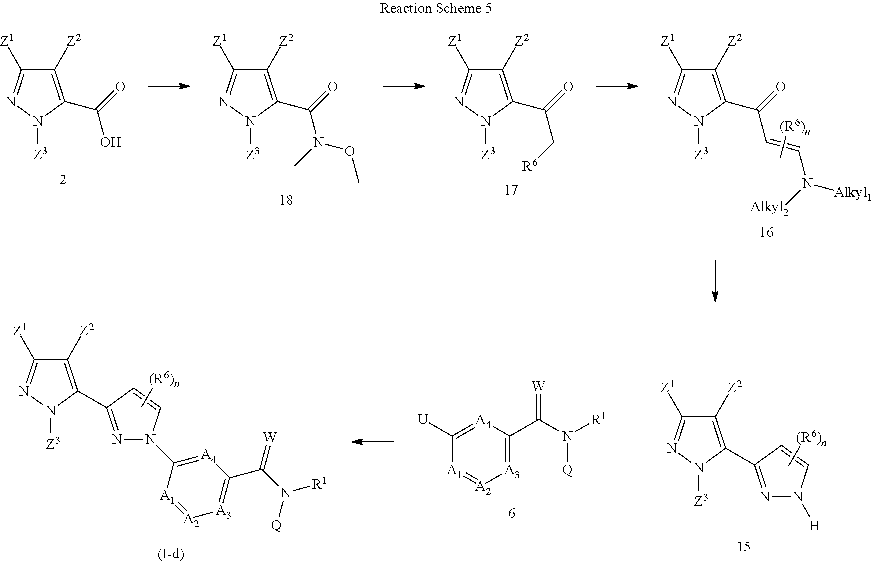

The radicals A₁-A₄, R¹, R⁶, Q, W and Z¹-Z³ have the meanings described above. U represents bromine, iodine, triflate, a boronic acid, a boronic ester or a trifluoroboronate. Compounds according to the invention of the general structure (I-d) can be prepared by processes known from the literature by reacting halogen or boron compounds of the general structure 6 with pyrazoles of the general structure 15 [see, for example, WO2011/59619; Tetrahedron 2011, 67, 5282-5288; WO2009/140342]. Pyrazoles of the general structure 15 can be prepared, for example, from enaminones of the general structure 16 and hydrazine compounds [see, for example, US2011/212949].

Enaminones of the general structure 16 can be prepared, for example, from ketones of the general formula 17, for example by reaction with dialkylamide dialkyl acetals [see, for example, WO2012/139930; US2011/212949]. Ketones of the general formula 17 can be prepared, for example, by reacting the corresponding Weinreb amides 18 with Grignard reagents [see, for example, WO2006/133885; US2010/222332]. For their part, the Weinreb amides of the general formula 18 can be prepared by processes known to the person skilled in the art from the corresponding carboxylic acids of the general structure 2.

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention thus encompasses pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. Preferably excluded from this are methods for the surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” also always encompasses the term “crop protection agent”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector. They can preferably be used as pesticides. They are effective against normally sensitive and resistant species and against all or some stages of development. The abovementioned pests include:

pests from the phylum of the Arthropoda, especially from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, for example Blatta orientalis, Blattella asahinai, Blattella germanica, Leucophaea maderae, Panchlora spp., Parcoblatta spp., Periplaneta spp., for example Periplaneta americana, Periplaneta australasiae, Supella longipalpa;
from the order of the Coleoptera, for example Acalymma vittatum, Acanthoscelides obtectus, Adoretus spp., Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Baris caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella, Lissorhoptrus oryzophilus, Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;
from the order of the Diptera, for example, Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta, Pegomya spp., for example Pegomya betae, Pegomya hyoscyami, Pegomya rubivora, Phlebotomus spp., Phorbia spp., Phormia spp., Piophila casei, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex;
from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma piri, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes citri, Diaphorina citri, Diaspis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium comi (=Parthenolecanium comi), Lepidosaphes spp., for example Lepidosaphes ulmi, Lipaphis erysimi, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Nephotettix spp., for example Nephotettix cincticeps, Nephotettix nigropictus, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla mali, Psylla pyri, Pteromalus spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxyacanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;
from the suborder of the Heteroptera, for example Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Monalonion atratum, Nezara spp., for example Nezara viridula, Oebalus spp., Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., for example Athalia rosae, Atta spp., Diprion spp., for example Diprion similis, Hoplocampa spp., for example Hoplocampa cookei, Hoplocampa testudinea, Lasius spp., Linepithema humile, Monomorium pharaonis, Sirex spp., Solenopsis invicta, Tapinoma spp., Urocerus spp., Vespa spp., for example Vespa crabro, Xeris spp.;
from the order of the Isopoda, for example Armadillidium vulgare, Oniscus asellus, Porcellio scaber;
from the order of the Isoptera, for example Coptotermes spp., for example Coptotermes formosanus, Cornitermes cumulans, Cryptotermes spp., Incisitermes spp., Microtermes obesi, Odontotermes spp., Reticulitermes spp., for example Reticulitermes flavipes, Reticulitermes hesperus;
from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diatraea saccharalis, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Etiella spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens, Hofmannophila pseudospretella, Homoeosoma spp., Homona spp., Hyponomeuta padella, Kakivoria flavofasciata, Laphygma spp., Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Oria spp., Orthaga spp., Ostrinia spp., for example Ostrinia nubilalis, Oulema melanopus, Oulema oryzae, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;
from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;
from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example Ceratophyllus spp., Ctenocephalides spp., for example Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Heliothrips spp., Hercinothrips femoralis, Rhipiphorothrips cruentatus, Scirtothrips spp., Taeniothrips cardamomi, Thrips spp., for example Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., for example Scutigerella immaculata;
pests from the phylum of the Mollusca, in particular from the class of the Bivalvia, for example Dreissena spp.;
and also from the class of the Gastropoda, for example Arion spp., for example Arion ater rufus, Biomphalaria spp., Bulinus spp., Deroceras spp., for example Deroceras laeve, Galba spp., Lymnaea spp., Oncomelania spp., Pomacea spp., Succinea spp.;
animal and human parasites from the phyla of the Platyhelminthes and Nematoda, for example Aelurostrongylus spp., Amidostomum spp., Ancylostoma spp., Angiostrongylus spp., Anisakis spp., Anoplocephala spp., Ascaris spp., Ascaridia spp., Baylisascaris spp., Brugia spp., Bunostomum spp., Capillaria spp., Chabertia spp., Clonorchis spp., Cooperia spp., Crenosoma spp., Cyathostoma spp., Dicrocoelium spp., Dictyocaulus spp., Diphyllobothrium spp., Dipylidium spp., Dirofilaria spp., Dracunculus spp., Echinococcus spp., Echinostoma spp., Enterobius spp., Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., Hyostrongylus spp., Litomosoides spp., Loa spp., Metastrongylus spp., Metorchis spp., Mesocestoides spp., Moniezia spp., Muellerius spp., Necator spp., Nematodirus spp., Nippostrongylus spp., Oesophagostomum spp., Ollulanus spp., Onchocerca spp., Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., Strongylus spp., Syngamus spp., Taenia spp., Teladorsagia spp., Thelazia spp., Toxascaris spp., Toxocara spp., Trichinella spp., Trichobilharzia spp., Trichostrongylus spp., Trichuris spp., Uncinaria spp., Wuchereria spp.;
plant pests from the phylum of the Nematoda, i.e. phytoparasitic nematodes, especially Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paraphelenchus spp., Paratrichodorus spp., for example Paratrichodorus minor, Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.
In addition, it is possible to control, from the sub-kingdom of the Protozoa, the order of the Coccidia, for example Eimeria spp.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). If appropriate, they can also be used as intermediates or precursors for the synthesis of other active compounds.

### Formulations

The present invention further relates to formulations and use forms prepared therefrom as pesticides, for example drench, drip and spray liquors, comprising at least one compound of the formula (I). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemically active compounds.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compounds of the formula (I) with auxiliaries, for example extenders, solvents and/or solid carriers and/or other auxiliaries, for example surfactants. The formulations are produced either in suitable facilities or else before or during application.

Auxiliaries used may be substances suitable for imparting special properties, such as particular physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, such as xylene, toluene or alkylnaphthalenes, chlorinated aromatic or chlorinated aliphatic hydrocarbons, such as chlorobenzene, chloroethylene or methylene chloride, aliphatic hydrocarbons, such as cyclohexane, paraffins, petroleum fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic minerals such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Especially suitable are those extenders or carriers which are gaseous at standard temperature and under standard pressure, for example aerosol propellants such as halogenated hydrocarbons, and also butane, propane, nitrogen and carbon dioxide.

Examples of emulsifiers and/or foam generators, dispersants or wetting agents having ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds comprising sulphates, sulphonates and phosphates, e.g. alkylaryl polyglycol ethers, alkylsulphonates, alkyl sulphates, arylsulphonates, protein hydrolysates, lignosulphite waste liquors, and methyl cellulose. The presence of a surfactant is advantageous when one of the compounds of the formula (I) and/or one of the inert carriers is insoluble in water and when the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethyl cellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

Optionally, further auxiliaries may be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Suitable penetrants in the present context are all those substances which are usually used for improving the penetration of agrochemical active compounds into plants. Penetrants are defined in this context by their ability to penetrate from the (generally aqueous) application liquor and/or from the spray coating into the cuticle of the plant and hence increase the mobility of the active compounds in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably contain between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (especially pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. The application is accomplished in a customary manner appropriate for the use forms.

### Mixtures

The compounds of the formula (I) can also be used in a mixture with one or more suitable fungicides, bactericides, acaricides, molluscicides, nematicides, insecticides, microbiologicals, beneficial organisms, herbicides, fertilizers, bird repellents, phytotonics, sterilants, safeners, semiochemicals and/or plant growth regulators, in order thus, for example, to broaden the spectrum of action, to prolong the duration of action, to increase the rate of action, to prevent repulsion or prevent evolution of resistance. In addition, active compound combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active compounds or semiochemicals such as attractants and/or bird repellents and/or plant activators and/or growth regulators and/or fertilizers. Likewise, the compounds of the formula (I) can be used in mixtures with agents to improve plant properties, for example growth, yield and quality of the harvested material.

In a particular embodiment of the invention, the compounds of the formula (I) are in the form of formulations or the use forms prepared from these formulations in a mixture with further compounds, preferably those as described below.

If one of the compounds mentioned below can occur in various tautomeric forms, these forms are also included even if not explicitly mentioned in each case.

### Insecticides/Acaricides/Nematicides

The active compounds specified here with their “common names” are known and are described for example in The Pesticide Manual, 16th ed., British Crop Protection Council 2012, or can be searched for on the Internet (e.g. http://www.alanwood.net/pesticides).

(1) Acetylcholinesterase (AChE) inhibitors, such as, for example, carbamates, for example alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel antagonists, for example cyclodiene-organochlorines, e.g. chlordane and endosulfan or phenylpyrazoles (fiproles), e.g. ethiprole and fipronil.
(3) Sodium channel modulators/voltage-gated sodium channel blockers, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrins (pyrethrum), resmethrin, silafluofen, tefluthrin, tetramethrin, tetramethrin [(1R) isomers], tralomethrin and transfluthrin or DDT or methoxychlor.
(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam or nicotine or sulfoxaflor.
(5) Allosteric activators of the nicotinergic acetylcholine receptor (nAChR), for example spinosyns, e.g. spinetoram and spinosad.
(6) Chloride channel activators, for example avermectins/milbemycins, e.g. abamectin, emamectin benzoate, lepimectin and milbemectin.
(7) Juvenile hormone imitators, for example, juvenile hormone analogues, e.g. hydroprene, kinoprene and methoprene or fenoxycarb or pyriproxyfen.
(8) Active compounds with unknown or nonspecific mechanisms of action, for example
alkyl halides, e.g. methyl bromide and other alkyl halides; or chloropicrine or sulphuryl fluoride or borax or tartar emetic.
(9) Selective antifeedants, e.g. pymetrozine or flonicamid.
(10) Mite growth inhibitors, e.g. clofentezine, hexythiazox and diflovidazin or etoxazole.
(11) Microbial disruptors of the insect gut membrane, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1Fa, Cry2Ab, mCry3A, Cry3Ab, Cry3Bb, Cry34/35Ab1.
(12) Oxidative phosphorylation inhibitors, ATP disruptors, for example diafenthiuron or organotin compounds, e.g. azocyclotin, cyhexatin and fenbutatin oxide or propargite or tetradifon.
(13) Oxidative phosphorylation decouplers that interrupt the H proton gradient, for example chlorfenapyr, DNOC and sulfluramid.
(14) Nicotinergic acetylcholine receptor antagonists, for example bensultap, cartap hydrochloride, thiocyclam, and thiosultap-sodium.
(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.
(16) Chitin biosynthesis inhibitors, type 1, for example buprofezin.
(17) Moulting inhibitors (in particular for Diptera, i.e. dipterans) such as, for example, cyromazine.
(18) Ecdysone receptor agonists, for example chromafenozide, halofenozide, methoxyfenozide and tebufenozide.
(19) Octopaminergic agonists, for example amitraz.
(20) Complex-III electron transport inhibitors, for example hydramethylnon or acequinocyl or fluacrypyrim.
(21) Complex-I electron transport inhibitors, for example METI acaricides, e.g. fenazaquin, fenpyroximate, pyrimidifen, pyridaben, tebufenpyrad and tolfenpyrad or rotenone (Derris).
(22) Voltage-gated sodium channel blockers, for example indoxacarb or metaflumizone.
(23) Inhibitors of acetyl-CoA carboxylase, for example tetronic and tetramic acid derivatives, e.g. spirodiclofen, spiromesifen and spirotetramat.
(24) Complex-IV electron transport inhibitors, for example phosphines, e.g. aluminium phosphide, calcium phosphide, phosphine and zinc phosphide or cyanide.
(25) Complex-II electron transport inhibitors, for example cyenopyrafen and cyflumetofen.
(28) Ryanodine receptor effectors, for example diamides, e.g. chlorantraniliprole, cyantraniliprole and flubendiamide,
further active compounds, for example afidopyropen, azadirachtin, benclothiaz, benzoximate, bifenazate, bromopropylate, chinomethionat, cryolite, dicofol, diflovidazin, fluensulfone, flometoquin, flufenerim, flufenoxystrobin, flufiprole, fluopyram, flupyradifurone, fufenozide, heptafluthrin, imidaclothiz, iprodione, meperfluthrin, paichongding, pyflubumide, pyrifluquinazon, pyriminostrobin, tetramethylfluthrin and iodomethane; and also preparations based on Bacillus firmus (I-I582, BioNeem, Votivo), and also the following compounds: 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) and 1-{2-fluoro-4-methyl-5-[(2,2,2-trifluoroethyl)sulphinyl]phenyl}-3-(trifluoromethyl)-1H-1,2,4-triazole-5-amine (known from WO2006/043635), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl-ethylcarbonate (known from WO2009/049851), 4-(but-2-yn-1-yloxy)-6-(3,5-dimethylpiperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS Reg. No. 1204776-60-2), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216), 4-{5-[3-chloro-5-(trifluoromethyl)phenyl]-5-(trifluoromethyl)-4,5-dihydro-1,2-oxazol-3-yl}-N-{2-oxo-2-[(2,2,2-trifluoroethyl)amino]ethyl}-1-naphthamide (known from WO2009/002809), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO2010/069502), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 3-chloro-N-(2-cyanopropan-2-yl)-N-[4-(1,1,1,2,3,3,3-heptafluoropropan-2-yl)-2-methylphenyl]phthalamide (known from WO2012/034472), 8-chloro-N-[(2-chloro-5-methoxyphenyl)sulphonyl]-6-(trifluoromethyl)imidazo[1,2-a]pyridine-2-carboxamide (known from WO2010/129500), 4-[5-(3,5-dichlorophenyl)-5-(trifluoromethyl)-4,5-dihydro-1,2-oxazol-3-yl]-2-methyl-N-(1-oxidothietan-3-yl)benzamide (known from WO2009/080250), N-[(2E)-1-[(6-chloropyridin-3-yl)methyl]pyridin-2(1H)-ylidene]-2,2,2-trifluoroacetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), (5S,8R)-1-[(6-chloropyridin-3-yl)methyl]-9-nitro-2,3,5,6,7,8-hexahydro-1H-5,8-epoxyimidazo[1,2-a]azepine (known from WO2010/069266), (2E)-1-[(6-chloropyridin-3-yl)methyl]-N′-nitro-2-pentylidenehydrazinecarboximidamide (known from WO2010/060231), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969).

### Fungicides

The active compounds specified herein by their common name are known and described, for example, in the “Pesticide Manual” or on the Internet (for example: http://www.alanwood.net/pesticides).

(1) Ergosterol biosynthesis inhibitors, for example (1.1) aldimorph, (1.2) azaconazole, (1.3) bitertanol, (1.4) bromuconazole, (1.5) cyproconazole, (1.6) diclobutrazole, (1.7) difenoconazole, (1.8) diniconazole, (1.9) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamid, (1.17) fenpropidin, (1.18) fenpropimorph, (1.19) fluquinconazole, (1.20) flurprimidol, (1.21) flusilazole, (1.22) flutriafole, (1.23) furconazole, (1.24) furconazole-cis, (1.25) hexaconazole, (1.26) imazalil, (1.27) imazalil sulphate, (1.28) imibenconazole, (1.29) ipconazole, (1.30) metconazole, (1.31) myclobutanil, (1.32) naftifin, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafin, (1.49) tetraconazole, (1.50) triadimefon, (1.51) triadimenol, (1.52) tridemorph, (1.53) triflumizole, (1.54) triforine, (1.55) triticonazole, (1.56) uniconazole, (1.57) uniconazole-P, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxylate, (1.62) N′-{5-(difluoromethyl)-2-methyl-4-[3-(trimethylsilyl)propoxy]phenyl}-N-ethyl-N-methylimidoformamide, (1.63) N-ethyl-N-methyl-N′-{2-methyl-5-(trifluoromethyl)-4-[3-(trimethylsilyl)propoxy]phenyl}imidoformamide and (1.64) O-[1-(4-methoxyphenoxy)-3,3-dimethylbutan-2-yl]-1H-imidazole-1-carbothioate, (1.65) pyrisoxazole.
(2) Respiration inhibitors (respiratory chain inhibitors), for example (2.1) bixafen, (2.2) boscalid, (2.3) carboxin, (2.4) diflumetorim, (2.5) fenfuram, (2.6) fluopyram, (2.7) flutolanil, (2.8) fluxapyroxad, (2.9) furametpyr, (2.10) furmecyclox, (2.11) isopyrazam mixture of the syn-epimeric racemate 1RS,4SR,9RS and the anti-epimeric racemate 1RS,4SR,9SR, (2.12) isopyrazam (anti-epimeric racemate), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27) N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazoline-4-amine, (2.29) benzovindiflupyr, (2.30) N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) isofetamid
(3) Respiration inhibitors (respiratory chain inhibitors) that act on complex III of the respiratory chain, for example (3.1) ametoctradin, (3.2) amisulbrom, (3.3) azoxystrobin, (3.4) cyazofamid, (3.5) coumethoxystrobin, (3.6) coumoxystrobin, (3.7) dimoxystrobin, (3.8) enestroburin, (3.9) famoxadone, (3.10) fenamidone, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylethanamide, (3.24) (2E)-2-(methoxyimino)-N-methyl-2-(2-{[({(1E)-1-[3-(trifluoromethyl)phenyl]ethylidene}amino)oxy]methyl}phenyl)ethanamide, (3.25) (2E)-2-(methoxyimino)-N-methyl-2-{2-[(E)-({1-[3-(trifluoromethyl)phenyl]ethoxy}imino)methyl]phenyl}ethanamide, (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide, (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulphanyl)methyl]phenyl}-3-methoxyprop-2-enoate, (3.31) N-(3-ethyl-3,5,5-trimethylcyclohexyl)-3-(formylamino)-2-hydroxybenzamide, (3.32) 2-{2-[(2,5-dimethylphenoxy)methyl]phenyl}-2-methoxy-N-methylacetamide,
(4) inhibitors of mitosis and cell division, for example (4.1) benomyl, (4.2) carbendazim, (4.3) chlorfenazole, (4.4) diethofencarb, (4.5) ethaboxam, (4.6) fluopicolid, (4.7) fuberidazole, (4.8) pencycuron, (4.9) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl)[1,2,4]triazolo[1,5-a]pyrimidine and (4.14) 3-chloro-5-(6-chloropyridin-3-yl)-6-methyl-4-(2,4,6-trifluorophenyl)pyridazine.
(5) Compounds having multisite activity such as, for example, (5.1) Bordeaux mixture, (5.2) captafol, (5.3) captan, (5.4) chlorothalonil, (5.5) copper preparations such as copper hydroxide, (5.6) copper naphthenate, (5.7) copper oxide, (5.8) copper oxychloride, (5.9) copper sulphate, (5.10) dichlofluanid, (5.11) dithianon, (5.12) dodine, (5.13) dodine free base, (5.14) ferbam, (5.15) fluorfolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) zinc metiram, (5.27) copper-oxine, (5.28) propamidine, (5.29) propineb, (5.30) sulphur and sulphur preparations such as, for example, calcium polysulphide, (5.31) thiram, (5.32) tolylfluanid, (5.33) zineb, (5.34) ziram and (5.35) anilazine.
(6) Resistance inducers, for example (6.1) acibenzolar-S-methyl, (6.2) isotianil, (6.3) probenazole, (6.4) tiadinil and (6.5) laminarin.
(7) Amino acid and protein biosynthesis inhibitors, for example (7.1), (7.2) blasticidin-S, (7.3) cyprodinil, (7.4) kasugamycin, (7.5) kasugamycin hydrochloride hydrate, (7.6) mepanipyrim, (7.7) pyrimethanil, (7.8) 3-(5-fluoro-3,3,4,4-tetramethyl-3,4-dihydroisoquinolin-1-yl)quinoline and (7.9) oxytetracycline and (7.10) streptomycin.
(8) ATP production inhibitors such as, for example, (8.1) fentin acetate, (8.2) fentin chloride, (8.3) fentin hydroxide and (8.4) silthiofam.
(9) Cell wall synthesis inhibitors, for example (9.1) benthiavalicarb, (9.2) dimethomorph, (9.3) flumorph, (9.4) iprovalicarb, (9.5) mandipropamid, (9.6) polyoxins, (9.7) polyoxorim, (9.8) validamycin A, (9.9) valifenalate and (9.10) polyoxin B.
(10) Lipid and membrane synthesis inhibitors, for example (10.1) biphenyl, (10.2) chlorneb, (10.3) dicloran, (10.4) edifenphos, (10.5) etridiazole, (10.6) iodocarb, (10.7) iprobenfos, (10.8) isoprothiolane, (10.9) propamocarb, (10.10) propamocarb hydrochloride, (10.11) prothiocarb, (10.12) pyrazophos, (10.13) quintozene, (10.14) tecnazene and (10.15) tolclofos-methyl.
(11) Melanin biosynthesis inhibitors, for example (11.1) carpropamid, (11.2) diclocymet, (11.3) fenoxanil, (11.4) fthalide, (11.5) pyroquilon, (11.6) tricyclazole and (11.7) 2,2,2-trifluoroethyl {3-methyl-1-[(4-methylbenzoyl)amino]butan-2-yl}carbamate.
(12) Nucleic acid synthesis inhibitors, for example (12.1) benalaxyl, (12.2) benalaxyl-M (kiralaxyl), (12.3) bupirimate, (12.4) clozylacon, (12.5) dimethirimol, (12.6) ethirimol, (12.7) furalaxyl, (12.8) hymexazole, (12.9) metalaxyl, (12.10) metalaxyl-M (mefenoxam), (12.11) ofurace, (12.12) oxadixyl, (12.13) oxolinic acid and (12.14) octhilinone.
(13) Signal transduction inhibitors, for example (13.1) chlozolinate, (13.2) fenpiclonil, (13.3) fludioxonil, (13.4) iprodione, (13.5) procymidone, (13.6) quinoxyfen, (13.7) vinclozolin and (13.8) proquinazid.
(14) Decouplers, for example (14.1) binapacryl, (14.2) dinocap, (14.3) ferimzone, (14.4) fluazinam and (14.5) meptyldinocap.
(15) Further compounds, for example (15.1) benthiazole, (15.2) bethoxazine, (15.3) capsimycin, (15.4) carvone, (15.5) quinomethionate, (15.6) pyriofenone (chlazafenone), (15.7) cufraneb, (15.8) cyflufenamid, (15.9) cymoxanil, (15.10) cyprosulfamide, (15.11) dazomet, (15.12) debacarb, (15.13) dichlorophen, (15.14) diclomezine, (15.15) difenzoquat, (15.16) difenzoquat methylsulphate, (15.17) diphenylamine, (15.18) EcoMate, (15.19) fenpyrazamine, (15.20) flumetover, (15.21) fluorimid, (15.22) flusulfamide, (15.23) flutianil, (15.24) fosetyl-aluminium, (15.25) fosetyl-calcium, (15.26) fosetyl-sodium, (15.27) hexachlorobenzene, (15.28) irumamycin, (15.29) methasulfocarb, (15.30) methyl isothiocyanate, (15.31) metrafenone, (15.32) mildiomycin, (15.33) natamycin, (15.34) nickel dimethyldithiocarbamate, (15.35) nitrothal-isopropyl, (15.36) octhilinone, (15.37) oxamocarb, (15.38) oxyfenthiin, (15.39) pentachlorophenol and its salts, (15.40) phenothrin, (15.41) phosphoric acid and its salts, (15.42) propamocarb-fosetylate, (15.43) propanosine-sodium, (15.44) pyrimorph, (15.45) (2E)-3-(4-tert-butylphenyl)-3-(2-chloropyridin-4-yl)-1-(morpholin-4-yl)prop-2-en-1-one, (15.46) (2Z)-3-(4-tert-butylphenyl)-3-(2-chloropyridin-4-yl)-1-(morpholin-4-yl)prop-2-en-1-one, (15.47) pyrrolnitrin, (15.48) tebufloquin, (15.49) tecloftalam, (15.50) tolnifanide, (15.51) triazoxide, (15.52) trichlamide, (15.53) zarilamid, (15.54) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (15.55) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.56) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.57) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.58) 1-(4-methoxyphenoxy)-3,3-dimethylbutan-2-yl 1H-imidazole-1-carboxylate, (15.59) 2,3,5,6-tetrachloro-4-(methylsulphonyl)pyridine, (15.60) 2,3-dibutyl-6-chlorothieno[2,3-d]pyrimidin-4(3H)-one, (15.61) 2,6-dimethyl-1H,5H-[1,4]dithiino[2,3-c:5,6-c′]dipyrrole-1,3,5,7(2H,6H)-tetrone, (15.62) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.63) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, (15.64) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.65) 2-butoxy-6-iodo-3-propyl-4H-chromen-4-one, (15.66) 2-chloro-5-[2-chloro-1-(2,6-difluoro-4-methoxyphenyl)-4-methyl-1H-imidazol-5-yl]pyridine, (15.67) 2-phenylphenol and salts, (15.68) 3-(4,4,5-trifluoro-3,3-dimethyl-3,4-dihydroisoquinolin-1-yl)quinoline, (15.69) 3,4,5-trichloropyridine-2,6-dicarbonitrile, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulphonohydrazide, (15.74) 5-fluoro-2-[(4-fluorobenzyl)oxy]pyrimidine-4-amine, (15.75) 5-fluoro-2-[(4-methylbenzyl)oxy]pyrimidine-4-amine, (15.76) 5-methyl-6-octyl[1,2,4]triazolo[1,5-a]pyrimidine-7-amine, (15.77) ethyl (2Z)-3-amino-2-cyano-3-phenylacrylate, (15.78) N′-(4-{[3-(4-chlorobenzyl)-1,2,4-thiadiazol-5-yl]oxy}-2,5-dimethylphenyl)-N-ethyl-N-methylimidoformamide, (15.79) N-(4-chlorobenzyl)-3-[3-methoxy-4-(prop-2-yn-1-yloxy)phenyl]propanamide, (15.80) N-[(4-chlorophenyl)(cyano)methyl]-3-[3-methoxy-4-(prop-2-yn-1-yloxy)phenyl]propanamide, (15.81) N-[(5-bromo-3-chloropyridin-2-yl)methyl]-2,4-dichloronicotinamide, (15.82) N-[1-(5-bromo-3-chloropyridin-2-yl)ethyl]-2,4-dichloronicotinamide, (15.83) N-[1-(5-bromo-3-chloropyridin-2-yl)ethyl]-2-fluoro-4-iodonicotinamide, (15.84) N-{(E)-[(cyclopropylmethoxy)imino][6-(difluoromethoxy)-2,3-difluorophenyl]methyl}-2-phenylacetamide, (15.85) N—{(Z)-[(cyclopropylmethoxy)imino][6-(difluoromethoxy)-2,3-difluorophenyl]methyl}-2-phenylacetamide, (15.86) N′-{4-[(3-tert-butyl-4-cyano-1,2-thiazol-5-yl)oxy]-2-chloro-5-methylphenyl}-N-ethyl-N-methylimidoformamide, (15.87) N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.88) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.89) N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, (15.90) pentyl {6-[({[(1-methyl-1H-tetrazol-5-yl)(phenyl)methylene]amino}oxy)methyl]pyridin-2-yl}carbamate, (15.91) phenazine-1-carboxylic acid, (15.92) quinolin-8-ol, (15.93) quinolin-8-ol sulphate (2:1), (15.94) tert-butyl {6-[({[(1-methyl-1H-tetrazol-5-yl)(phenyl)methylene]amino}oxy)methyl]pyridin-2-yl}carbamate, (15.95) 1-methyl-3-(trifluoromethyl)-N-[2′-(trifluoromethyl)biphenyl-2-yl]-1H-pyrazole-4-carboxamide, (15.96) N-(4′-chlorobiphenyl-2-yl)-3-(difluoromethyl)-1-methyl-1H-pyrazole-4-carboxamide, (15.97) N-(2′,4′-dichlorobiphenyl-2-yl)-3-(difluoromethyl)-1-methyl-1H-pyrazole-4-carboxamide, (15.98) 3-(difluoromethyl)-1-methyl-N-[4′-(trifluoromethyl)biphenyl-2-yl]-1H-pyrazole-4-carboxamide, (15.99) N-(2′,5′-difluorobiphenyl-2-yl)-1-methyl-3-(trifluoromethyl)-1H-pyrazole-4-carboxamide, (15.100) 3-(difluoromethyl)-1-methyl-N-[4′-(prop-1-yn-1-yl)biphenyl-2-yl]-1H-pyrazole-4-carboxamide, (15.101) 5-fluoro-1,3-dimethyl-N-[4′-(prop-1-yn-1-yl)biphenyl-2-yl]-1H-pyrazole-4-carboxamide, (15.102) 2-chloro-N-[4′-(prop-1-yn-1-yl)biphenyl-2-yl]nicotinamide, (15.103) 3-(difluoromethyl)-N-[4′-(3,3-dimethylbut-1-yn-1-yl)biphenyl-2-yl]-1-methyl-1H-pyrazole-4-carboxamide, (15.104) N-[4′-(3,3-dimethylbut-1-yn-1-yl)biphenyl-2-yl]-5-fluoro-1,3-dimethyl-1H-pyrazole-4-carboxamide, (15.105) 3-(difluoromethyl)-N-(4′-ethynylbiphenyl-2-yl)-1-methyl-1H-pyrazole-4-carboxamide, (15.106) N-(4′-ethynylbiphenyl-2-yl)-5-fluoro-1,3-dimethyl-1H-pyrazole-4-carboxamide, (15.107) 2-chloro-N-(4′-ethynylbiphenyl-2-yl)nicotinamide, (15.108) 2-chloro-N-[4′-(3,3-dimethylbut-1-yn-1-yl)biphenyl-2-yl]nicotinamide, (15.109) 4-(difluoromethyl)-2-methyl-N-[4′-(trifluoromethyl)biphenyl-2-yl]-1,3-thiazole-5-carboxamide, (15.110) 5-fluoro-N-[4′-(3-hydroxy-3-methylbut-1-yn-1-yl)biphenyl-2-yl]-1,3-dimethyl-1H-pyrazole-4-carboxamide, (15.111) 2-chloro-N-[4′-(3-hydroxy-3-methylbut-1-yn-1-yl)biphenyl-2-yl]nicotinamide, (15.112) 3-(difluoromethyl)-N-[4′-(3-methoxy-3-methylbut-1-yn-1-yl)biphenyl-2-yl]-1-methyl-1H-pyrazole-4-carboxamide, (15.113) 5-fluoro-N-[4′-(3-methoxy-3-methylbut-1-yn-1-yl)biphenyl-2-yl]-1,3-dimethyl-1H-pyrazole-4-carboxamide, (15.114) 2-chloro-N-[4′-(3-methoxy-3-methylbut-1-yn-1-yl)biphenyl-2-yl]nicotinamide, (15.115) (5-bromo-2-methoxy-4-methylpyridin-3-yl)(2,3,4-trimethoxy-6-methylphenyl)methanone, (15.116) N-[2-(4-{[3-(4-chlorophenyl)prop-2-yn-1-yl]oxy}-3-methoxyphenyl)ethyl]-N2-(methylsulphonyl)valinamide, (15.117) 4-oxo-4-[(2-phenylethyl)amino]butanoic acid, (15.118) but-3-yn-1-yl {6-[({[(Z)-(1-methyl-1H-tetrazol-5-yl)(phenyl)methylene]amino}oxy)methyl]pyridin-2-yl}carbamate, (15.119) 4-amino-5-fluoropyrimidin-2-ol (tautomeric form: 4-amino-5-fluoropyrimidin-2(1H)-one), (15.120) propyl 3,4,5-trihydroxybenzoate, (15.121) 1,3-dimethyl-N-(1,1,3-trimethyl-2,3-dihydro-1H-inden-4-yl)-1H-pyrazole-4-carboxamide, (15.122) 1,3-dimethyl-N-[(3R)-1,1,3-trimethyl-2,3-dihydro-1H-inden-4-yl]-1H-pyrazole-4-carboxamide, (15.123) 1,3-dimethyl-N-[(3S)-1,1,3-trimethyl-2,3-dihydro-1H-inden-4-yl]-1H-pyrazole-4-carboxamide, (15.124) [3-(4-chloro-2-fluorophenyl)-5-(2,4-difluorophenyl)-1,2-oxazol-4-yl](pyridin-3-yl)methanol, (15.125) (S)-[3-(4-chloro-2-fluorophenyl)-5-(2,4-difluorophenyl)-1,2-oxazol-4-yl](pyridin-3-yl)methanol, (15.126) (R)-[3-(4-chloro-2-fluorophenyl)-5-(2,4-difluorophenyl)-1,2-oxazol-4-yl](pyridin-3-yl)methanol, (15.127) 2-{[3-(2-chlorophenyl)-2-(2,4-difluorophenyl)oxiran-2-yl]methyl}-2,4-dihydro-3H-1,2,4-triazole-3-thione, (15.128) 1-{[3-(2-chlorophenyl)-2-(2,4-difluorophenyl)oxiran-2-yl]methyl}-1H-1,2,4-triazol-5-yl thiocyanate, (15.129) 5-(allylsulphanyl)-1-{[3-(2-chlorophenyl)-2-(2,4-difluorophenyl)oxiran-2-yl]methyl}-1H-1,2,4-triazole, (15.130) 2-[1-(2,4-dichlorophenyl)-5-hydroxy-2,6,6-trimethylheptan-4-yl]-2,4-dihydro-3H-1,2,4-triazole-3-thione, (15.131) 2-{[rel(2R,3 S)-3-(2-chlorophenyl)-2-(2,4-difluorophenyl)oxiran-2-yl]methyl}-2,4-dihydro-3H-1,2,4-triazole-3-thione, (15.132) 2-{[rel(2R,3R)-3-(2-chlorophenyl)-2-(2,4-difluorophenyl)oxiran-2-yl]methyl}-2,4-dihydro-3H-1,2,4-triazole-3-thione, (15.133) 1-{[rel(2R,3S)-3-(2-chlorophenyl)-2-(2,4-difluorophenyl)oxiran-2-yl]methyl}-1H-1,2,4-triazol-5-yl thiocyanate, (15.134) 1-{[rel(2R,3R)-3-(2-chlorophenyl)-2-(2,4-difluorophenyl)oxiran-2-yl]methyl}-H-1,2,4-triazol-5-yl thiocyanate, (15.135) 5-(allylsulphanyl)-1-{[rel(2R,3 S)-3-(2-chlorophenyl)-2-(2,4-difluorophenyl)oxiran-2-yl]methyl}-1H-1,2,4-triazole, (15.136) 5-(allylsulphanyl)-1-{[rel(2R,3R)-3-(2-chlorophenyl)-2-(2,4-difluorophenyl)oxiran-2-yl]methyl}-1H-1,2,4-triazole, (15.137) 2-[(2S,4S,5S)-1-(2,4-dichlorophenyl)-5-hydroxy-2,6,6-trimethylheptan-4-yl]-2,4-dihydro-3H-1,2,4-triazole-3-thione, (15.138) 2-[(2R,4S,5S)-1-(2,4-dichlorophenyl)-5-hydroxy-2,6,6-trimethylheptan-4-yl]-2,4-dihydro-3H-1,2,4-triazole-3-thione, (15.139) 2-[(2R,4R,5R)-1-(2,4-dichlorophenyl)-5-hydroxy-2,6,6-trimethylheptan-4-yl]-2,4-dihydro-3H-1,2,4-triazole-3-thione, (15.140) 2-[(2S,4R,5R)-1-(2,4-dichlorophenyl)-5-hydroxy-2,6,6-trimethylheptan-4-yl]-2,4-dihydro-3H-1,2,4-triazole-3-thione, (15.141) 2-[(2S,4S,5R)-1-(2,4-dichlorophenyl)-5-hydroxy-2,6,6-trimethylheptan-4-yl]-2,4-dihydro-3H-1,2,4-triazole-3-thione, (15.142) 2-[(2R,4S,5R)-1-(2,4-dichlorophenyl)-5-hydroxy-2,6,6-trimethylheptan-4-yl]-2,4-dihydro-3H-1,2,4-triazole-3-thione, (15.143) 2-[(2R,4R,5S)-1-(2,4-dichlorophenyl)-5-hydroxy-2,6,6-trimethylheptan-4-yl]-2,4-dihydro-3H-1,2,4-triazole-3-thione, (15.144) 2-[(2S,4R,5S)-1-(2,4-dichlorophenyl)-5-hydroxy-2,6,6-trimethylheptan-4-yl]-2,4-dihydro-3H-1,2,4-triazole-3-thione, (15.145) 2-fluoro-6-(trifluoromethyl)-N-(1,1,3-trimethyl-2,3-dihydro-1H-inden-4-yl)benzamide, (15.146) 2-(6-benzylpyridin-2-yl)quinazoline, (15.147) 2-[6-(3-fluoro-4-methoxyphenyl)-5-methylpyridin-2-yl]quinazoline, (15.148) 3-(4,4-difluoro-3,3-dimethyl-3,4-dihydroisoquinolin-1-yl)quinoline, (15.149) abscisic acid, (15.150) 3-(difluoromethyl)-N-methoxy-1-methyl-N-[1-(2,4,6-trichlorophenyl)propan-2-yl]-1H-pyrazole-4-carboxamide, (15.151) N′-[5-bromo-6-(2,3-dihydro-1H-inden-2-yloxy)-2-methylpyridin-3-yl]-N-ethyl-N-methylimidoformamide, (15.152) N′-{5-bromo-6-[1-(3,5-difluorophenyl)ethoxy]-2-methylpyridin-3-yl}-N-ethyl-N-methylimidoformamide, (15.153) N′-{5-bromo-6-[(1R)-1-(3,5-difluorophenyl)ethoxy]-2-methylpyridin-3-yl}-N-ethyl-N-methylimidoformamide, (15.154) N′-{5-bromo-6-[(1S)-1-(3,5-difluorophenyl)ethoxy]-2-methylpyridin-3-yl}-N-ethyl-N-methylimidoformamide, (15.155) N′-{5-bromo-6-[(cis-4-isopropylcyclohexyl)oxy]-2-methylpyridin-3-yl}-N-ethyl-N-methylimidoformamide, (15.156) N′-{5-bromo-6-[(trans-4-isopropylcyclohexyl)oxy]-2-methylpyridin-3-yl}-N-ethyl-N-methylimidoformamide, (15.157) N-cyclopropyl-3-(difluoromethyl)-5-fluoro-N-(2-isopropylbenzyl)-1-methyl-1H-pyrazole-4-carboxamide, (15.158) N-cyclopropyl-N-(2-cyclopropylbenzyl)-3-(difluoromethyl)-5-fluoro-1-methyl-1H-pyrazole-4-carboxamide, (15.159) N-(2-tert-butylbenzyl)-N-cyclopropyl-3-(difluoromethyl)-5-fluoro-1-methyl-1H-pyrazole-4-carboxamide, (15.160) N-(5-chloro-2-ethylbenzyl)-N-cyclopropyl-3-(difluoromethyl)-5-fluoro-1-methyl-1H-pyrazole-4-carboxamide, (15.161) N-(5-chloro-2-isopropylbenzyl)-N-cyclopropyl-3-(difluoromethyl)-5-fluoro-1-methyl-1H-pyrazole-4-carboxamide, (15.162) N-cyclopropyl-3-(difluoromethyl)-N-(2-ethyl-5-fluorobenzyl)-5-fluoro-1-methyl-1H-pyrazole-4-carboxamide, (15.163) N-cyclopropyl-3-(difluoromethyl)-5-fluoro-N-(5-fluoro-2-isopropylbenzyl)-1-methyl-1H-pyrazole-4-carboxamide, (15.164) N-cyclopropyl-N-(2-cyclopropyl-5-fluorobenzyl)-3-(difluoromethyl)-5-fluoro-1-methyl-1H-pyrazole-4-carboxamide, (15.165) N-(2-cyclopentyl-5-fluorobenzyl)-N-cyclopropyl-3-(difluoromethyl)-5-fluoro-1-methyl-1H-pyrazole-4-carboxamide, (15.166) N-cyclopropyl-3-(difluoromethyl)-5-fluoro-N-(2-fluoro-6-isopropylbenzyl)-1-methyl-1H-pyrazole-4-carboxamide, (15.167) N-cyclopropyl-3-(difluoromethyl)-N-(2-ethyl-5-methylbenzyl)-5-fluoro-1-methyl-1H-pyrazole-4-carboxamide, (15.168) N-cyclopropyl-3-(difluoromethyl)-5-fluoro-N-(2-isopropyl-5-methylbenzyl)-1-methyl-1H-pyrazole-4-carboxamide, (15.169) N-cyclopropyl-N-(2-cyclopropyl-5-methylbenzyl)-3-(difluoromethyl)-5-fluoro-1-methyl-1H-pyrazole-4-carboxamide, (15.170) N-(2-tert-butyl-5-methylbenzyl)-N-cyclopropyl-3-(difluoromethyl)-5-fluoro-1-methyl-1H-pyrazole-4-carboxamide, (15.171) N-[5-chloro-2-(trifluoromethyl)benzyl]-N-cyclopropyl-3-(difluoromethyl)-5-fluoro-1-methyl-1H-pyrazole-4-carboxamide, (15.172) N-cyclopropyl-3-(difluoromethyl)-5-fluoro-1-methyl-N-[5-methyl-2-(trifluoromethyl)benzyl]-1H-pyrazole-4-carboxamide, (15.173) N-[2-chloro-6-(trifluoromethyl)benzyl]-N-cyclopropyl-3-(difluoromethyl)-5-fluoro-1-methyl-1H-pyrazole-4-carboxamide, (15.174) N-[3-chloro-2-fluoro-6-(trifluoromethyl)benzyl]-N-cyclopropyl-3-(difluoromethyl)-5-fluoro-1-methyl-1H-pyrazole-4-carboxamide, (15.175) N-cyclopropyl-3-(difluoromethyl)-N-(2-ethyl-4,5-dimethylbenzyl)-5-fluoro-1-methyl-1H-pyrazole-4-carboxamide, (15.176) N-cyclopropyl-3-(difluoromethyl)-5-fluoro-N-(2-isopropylbenzyl)-1-methyl-1H-pyrazol-4-carbothioamide, (15.177) 3-(difluoromethyl)-N-(7-fluoro-1,1,3-trimethyl-2,3-dihydro-1H-inden-4-yl)-1-methyl-1H-pyrazole-4-carboxamide, (15.178) 3-(difluoromethyl)-N-[(3R)-7-fluoro-1,1,3-trimethyl-2,3-dihydro-1H-inden-4-yl]-1-methyl-1H-pyrazole-4-carboxamide, (15.179) 3-(difluoromethyl)-N-[(3S)-7-fluoro-1,1,3-trimethyl-2,3-dihydro-1H-inden-4-yl]-1-methyl-1H-pyrazole-4-carboxamide, (15.180) N′-(2,5-dimethyl-4-phenoxyphenyl)-N-ethyl-N-methylimidoformamide, (15.181) N′-{4-[(4,5-dichloro-1,3-thiazol-2-yl)oxy]-2,5-dimethylphenyl}-N-ethyl-N-methylimidoformamide, (15.182) N-(4-chloro-2,6-difluorophenyl)-4-(2-chloro-4-fluorophenyl)-1,3-dimethyl-1H-pyrazole-5-amine. All the mixing components mentioned in classes (1) to (15), as the case may be, may form salts with suitable bases or acids if they are capable of doing so on the basis of their functional groups.

### Biological Pesticides as Mixing Components

The compounds of the formula (I) can be combined with biological pesticides.

Biological pesticides include especially bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, in particular B. cereus strain CNCM I-I562 or Bacillus firmus, strain I-I582 (Accession number CNCM I-I582) or Bacillus pumilus, in particular strain GB34 (Accession No. ATCC 700814) and strain QST2808 (Accession No. NRRL B-30087), or Bacillus subtilis, in particular strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, in particular B. thuringiensis subspecies israelensis (serotype H-14), strain AM65-52 (Accession No. ATCC 1276), or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).
Examples of fungi and yeasts which are used or can be used as biological pesticides are:
Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (now: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39. (Accession Number CNCM I-952).
Examples of viruses which are used or can be used as biological pesticides are:
Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.
Also included are bacteria and fungi which are added as ‘inoculant’ to plants or plant parts or plant organs and which, by virtue of their particular properties, promote plant growth and plant health. Examples include:
Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and parts of plants can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya bean, potato, sugar beet, sugar cane, tomatoes, peas and other vegetable species, cotton, tobacco, oilseed rape, and also fruit plants (with the fruits apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested material and vegetative and generative propagation material, for example cuttings, tubers, rhizomes, slips and seeds.

The inventive treatment of the plants and parts of plants with the compounds of the formula (I) is effected directly or by allowing them to act on the surroundings, habitat or storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible in accordance with the invention to treat all plants and parts thereof. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been grown by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful traits to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better storage life and/or processability of the harvested products. Further and particularly emphasized examples of such properties are increased resistance of the plants against animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants against phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain active herbicidal ingredients, for example imidazolinones, sulphonylureas, glyphosates or phosphinothricin (for example the “PAT” gene). The genes which impart the desired properties (“traits”) in question may also be present in combinations with one another in the transgenic plants. Examples of transgenic plants include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapevines), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection—Types of Treatment

The treatment of the plants and plant parts with the compounds of the formula (I) is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, meaning that the compounds of the formula (I) are applied to the foliage, where treatment frequency and application rate should be adjusted according to the level of infestation with the pest in question.

In the case of systemically active compounds, the compounds of the formula (I) also get into the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvement. Nevertheless, the treatment of seed gives rise to a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active compound used so as to provide optimum protection for the seed and the germinating plant from attack by animal pests, but without damage to the plant itself by the active compound used. In particular, methods for the treatment of seed should also take account of the intrinsic insecticidal or nematicidal properties of pest-resistant or -tolerant transgenic plants in order to achieve optimal protection of the seed and the germinating plant with a minimum expenditure of pesticides.

The present invention therefore also relates, more particularly, to a method for protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It also further comprises a method where the seed is treated at different times with a compound of the formula (I) and a mixing component.

The invention likewise relates to the use of the compounds of the formula (I) for treatment of seed for protection of the seed and the resulting plant from animal pests.

The invention further relates to seed which has been treated with a compound of the formula (I) for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occur when one of the compounds of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. More particularly, this includes seed of cereals (for example wheat, barley, rye, millet and oats), maize, cotton, soya beans, rice, potatoes, sunflowers, coffee, tobacco, canola, oilseed rape, beet (for example sugar beet and fodder beet), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya bean, cotton, canola, oilseed rape and rice.

As already mentioned above, the treatment of transgenic seed with a compound of the formula (I) is also of particular significance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichorma, Clavibacter, Glomus or Gliocladium. The present invention is particularly suitable for the treatment of transgenic seed containing at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been imbibed in water up to a certain stage (pigeon breast stage) for example, which leads to improved germination and more uniform emergence.

In general, in the treatment of the seed, it has to be ensured that the amount of the compound of the formula (I) and/or further additives applied to the seed is chosen such that the germination of the seed is not impaired and the plant which arises therefrom is not damaged. This has to be ensured particularly in the case of active compounds which can exhibit phytotoxic effects at certain application rates.

The compounds of the formula (I) are generally applied to the seed in a suitable formulation. Suitable formulations and processes for seed treatment are known to the person skilled in the art.

The compounds of the formula (I) can be converted to the customary seed dressing formulations, such as solutions, emulsions, suspensions, powders, foams, slurries or other coating compositions for seed, and also ULV formulations.

These formulations are produced in a known manner, by mixing the compounds of the formula (I) with customary additives, for example customary extenders and solvents or diluents, dyes, wetters, dispersants, emulsifiers, antifoams, preservatives, secondary thickeners, stickers, gibberellins and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetters which may be present in the seed dressing formulations usable in accordance with the invention are all substances which promote wetting and which are conventionally used for the formulation of active agrochemical ingredients. Preference is given to using alkyl naphthalenesulphonates, such as diisopropyl or diisobutyl naphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate/formaldehyde condensates.

Antifoams which may be present in the seed dressing formulations usable in accordance with the invention are all foam-inhibiting substances conventionally used for formulation of active agrochemical ingredients. Silicone antifoams and magnesium stearate can be used with preference.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed dressing formulations usable in accordance with the invention are all customary binders usable in seed dressing products. Preferred examples include polyvinylpyrrolidone, polyvinyl acetate, polyvinyl alcohol and tylose.

Gibberellins which may be present in the seed dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz-und Schädlingsbekämpfungsmittel”, vol. 2, Springer Verlag, 1970, pp. 401-412).

The seed dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats, and triticale, and also the seed of maize, rice, oilseed rape, peas, beans, cotton, sunflowers, soya beans and beets, or else a wide variety of different vegetable seed. The seed dressing formulations usable in accordance with the invention, or the dilute use forms thereof, can also be used to dress seed of transgenic plants.

For treatment of seed with the seed dressing formulations usable in accordance with the invention, or the use forms prepared therefrom, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the field of animal health, i.e. in the field of veterinary medicine, the compounds of the formula (I) are active against animal parasites, in particular ectoparasites or endoparasites. The term “endoparasites” includes especially helminths and protozoans, such as coccidia. Ectoparasites are typically and preferably arthropods, especially insects and acarids.

In the field of veterinary medicine, the compounds of the formula (I) having favourable homeotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals such as sheep, goats, horses, donkeys, camels, buffalo, rabbits, reindeer, fallow deer, and particularly cattle and pigs; poultry such as turkeys, ducks, geese, and particularly chickens; fish and crustaceans, for example in aquaculture, and also insects such as bees.

Domestic animals include, for example, mammals, such as hamsters, guinea pigs, rats, mice, chinchillas, ferrets, and particularly dogs, cats, caged birds, reptiles, amphibians and aquarium fish.

In a preferred embodiment, the compounds of the formula (I) are administered to mammals.

In another preferred embodiment, the compounds of the formula (I) are administered to birds, namely caged birds and particularly poultry.

Use of the compounds of the formula (I) for the control of animal parasites is intended to reduce or prevent illness, cases of deaths and reductions in performance (in the case of meat, milk, wool, hides, eggs, honey and the like), such that more economical and simpler animal keeping is enabled and better animal well-being is achievable.

In relation to the animal health field, the term “control” or “controlling” means that the compounds of the formula (I) are effective in reducing the incidence of the particular parasite in an animal infected with such parasites to an innocuous degree. More specifically, “controlling” in the present context means that the compound of the formula (I) can kill the respective parasite, inhibit its growth, or inhibit its proliferation.

Arthropods include:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hyporma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.;
from the order of the Heteropterida, for example Cimex spp., Triatoma spp., Rhodnius spp., Panstrongylus spp.; and also nuisance and hygiene pests from the order of the Blattarida.

Arthropods further include:

from the subclass of the Acari (Acarina) and the order of the Metastigmata, for example from the family of Argasidae like Argas spp., Ornithodorus spp., Otobius spp., from the family of Ixodidae like Ixodes spp., Amblyomma spp., Rhipicephalus (Boophilus) spp., Dermacentor spp., Haemophysalis spp., Hyalomma spp., Rhipicephalus spp. (the original genus of multi-host ticks); from the order of Mesostigmata like Dermanyssus spp., Omithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Parasitic Protozoa include:

Mastigophora (Flagellata), for example Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example, Giardia lamblia, G. canis;
Sarcomastigophora (Rhizopoda) such as Entamoebidae, for example, Entamoeba histolytica, Hartmanellidae, for example, Acanthamoeba sp., Harmanella sp;
Apicomplexa (Sporozoa) such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example Toxoplasma gondii, Hammondia heydornii, Neospora caninum, Besnoitia besnoitii; such as Sarcocystidae, for example Sarcocystis bovicanis, S. bovihominis, S. ovicanis, S. ovifelis, S. neurona, S. spec., S. suihominis, such as Leucozoidae, for example Leucozytozoon simondi, such as Plasmodiidae, for example Plasmodium berghei, P. falciparum, P. malariae, P. ovale, P. vivax, P. spec., such as Piroplasmea, for example Babesia argentina, B. bovis, B. canis, B. spec., Theileria parva, Theileria spec., such as Adeleina, for example Hepatozoon canis, H. spec.

Pathogenic endoparasites which are helminths include Platyhelmintha (e.g. Monogenea, cestodes and trematodes), nematodes, Acanthocephala, and Pentastoma. These include:

Monogenea: for example: Gyrodactylus spp., Dactylogyrus spp., Polystoma spp;
Cestodes: from the order of the Pseudophyllidea for example: Diphyllobothrium spp., Spirometra spp., Schistocephalus spp., Ligula spp., Bothridium spp., Diplogonoporus spp;
from the order of the Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp;
Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hyporaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.;
Nematodes: Trichinellida, for example: Trichuris spp., Capillaria spp., Paracapillaria spp., Eucoleus spp., Trichomosoides spp., Trichinella spp.;
from the order of the Tylenchida, for example: Micronema spp., Strongyloides spp.;
from the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;
from the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;
Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.;
from the order of the Echinorhynchida, for example, Acanthocephalus spp., Echinorhynchus spp., Leptorhynchoides spp;
Pentastoma: from the order of the Porocephalida, for example, Linguatula spp.

In the veterinary field and in animal keeping, the compounds of the formula (I) are administered by methods generally known in the art, such as via the enteral, parenteral, dermal or nasal route in the form of suitable preparations. Administration may be prophylactic or therapeutic.

Thus, one embodiment of the present invention refers to the use of a compound of the formula (I) as a medicament.

A further aspect refers to the use of a compound of the formula (I) as an antiendoparasitic agent, in particular a helminthicidal agent or antiprotozoic agent. Compounds of the formula (I) are suitable for use as an antiendoparasitic agent, especially as a helminthicidal agent or antiprotozoic agent, for example in animal breeding, in animal husbandry, in animal houses and in the hygiene sector.

A further aspect in turn relates to the use of a compound of the formula (I) as an antiectoparasitic, in particular an arthropodicide such as an insecticide or an acaricide. A further aspect relates to the use of a compound of the formula (I) as an antiectoparasitic, in particular an arthropodicide such as an insecticide or an acaricide, for example in animal husbandry, in animal breeding, in animal houses or in the hygiene sector.

### Vector Control

The compounds of the formula (I) can also be used in vector control. In the context of the present invention, a vector is an arthropod, especially an insect or arachnid, capable of transmitting pathogens, for example, viruses, worms, single-cell organisms and bacteria, from a reservoir (plant, animal, human, etc.) to a host. The pathogens can be transmitted either mechanically (for example trachoma by non-stinging flies) to a host or after injection (for example malaria parasites by mosquitoes) into a host.

Examples of vectors and the diseases or pathogens they transmit are:

### 1) Mosquitoes

Anopheles: malaria, filariasis;

Culex: Japanese encephalitis, filariasis, other viral diseases, transmission of worms;

Aedes: yellow fever, dengue fever, filariasis, other viral diseases;

Simulidae: transmission of worms, in particular Onchocerca volvulus;

2) Lice: skin infections, epidemic typhus;
3) Fleas: plague, endemic typhus;
4) Flies: sleeping sickness (trypanosomiasis); cholera, other bacterial diseases;
5) Mites: acariosis, epidemic typhus, rickettsialpox, tularaemia, Saint Louis encephalitis, tick-borne encephalitis (TBE), Crimean-Congo haemorrhagic fever, borreliosis;
6) Ticks: borellioses such as Borrelia duttoni, tick-borne encephalitis, Q fever (Coxiella burnetii), babesioses (Babesia canis canis).

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forestry, in gardens and in leisure facilities, and also in the protection of materials and stored products.

### Protection of Industrial Materials

The compounds of the formula (I) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) are in the form of a ready-to-use pesticide, meaning that they can be applied to the material in question without further modifications. Suitable further insecticides or fungicides are in particular those mentioned above.

It has also been found that, surprisingly, the compounds of the formula (I) can be used to protect objects which come into contact with saltwater or brackish water, especially hulls, screens, nets, buildings, moorings and signalling systems, against fouling. It is equally possible to use the compounds of the formula (I), alone or in combinations with other active compounds, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids and mites encountered in enclosed spaces, for example dwellings, factory halls, offices, vehicle cabins. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

### Experimental Part

### Preparation of N-{2-fluoro-5-[2′-methyl-5′-(pentafluoroethyl)-4′-(trifluoromethyl)-2′H-1,3′-bipyrazol-4-yl]benzyl}propanamide (Ic-1)

Reaction Scheme 6 shows the synthesis of the compound Ic-1 according to the invention.

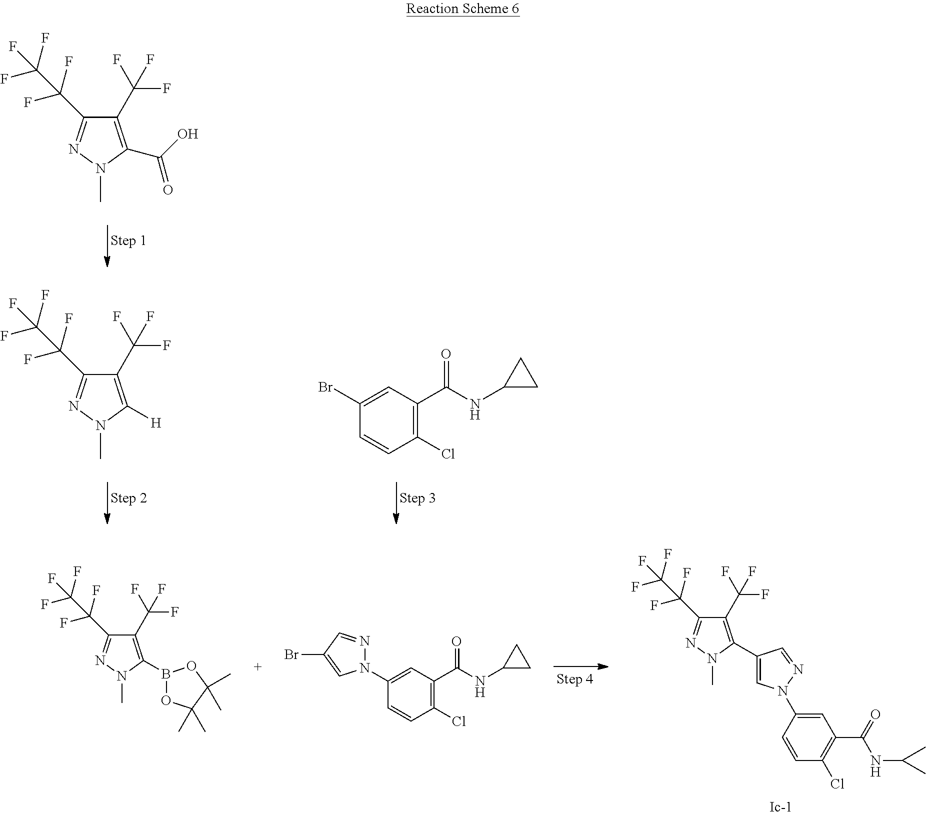 [SMILES: Cn1nc(C(F)(F)C(F)(F)F)c(C(F)(F)F)c1C(=O)O ; Cn1cc(C(F)(F)F)c(C(F)(F)C(F)(F)F)n1 ; O=C(NC1CC1)c1cc(Br)ccc1Cl ; Cn1nc(C(F)(F)C(F)(F)F)c(C(F)(F)F)c1B1OC(C)(C)C(C)(C)O1 ; O=C(NC1CC1)c1cc(-n2cc(Br)cn2)ccc1Cl ; CC(C)NC(=O)c1cc(-n2cc(-c3c(C(F)(F)F)c(C(F)(F)C(F)(F)F)nn3C)cn2)ccc1Cl]

### Step 1: Synthesis of 1-methyl-3-(1,1,2,2,2-pentafluoroethyl)-4-(trifluoromethyl)pyrazole

19.7 g (240 mmol) of sodium acetate are added to a solution of 50.0 g (160 mmol) of 1-methyl-3-(pentafluoroethyl)-4-(trifluoromethyl)-1H-pyrazole-5-carboxylic acid in 200 ml of dimethyl sulphoxide and 50 ml of water. The reaction mixture is stirred at 100° C. overnight, then cooled to room temperature, diluted with tert-butyl methyl ether and washed successively with saturated aqueous sodium carbonate solution and saturated aqueous sodium chloride solution. The organic phase is dried over magnesium sulphate, filtered and concentrated on a rotary evaporator under reduced pressure. This gives 40.5 g of an orange liquid as a crude product.

The reaction is repeated on the same scale, giving an additional 40.7 g of an orange oil as a crude product.

The crude products are combined and purified by distillation (17 mbar, about 72° C.).

This gives 77.3 g of 1-methyl-3-(1,1,2,2,2-pentafluoroethyl)-4-(trifluoromethyl)pyrazole as a slightly yellow liquid.

¹H-NMR (400 MHz, d₆-DMSO): δ=8.69 (s, 1H), 4.00 (s, 3H)

HPLC-MS: log P ^{a)}=3.23.

GC-MS: mass (m/z)=268.0 [M]⁺.

### Step 2: Synthesis of 1-methyl-3-(1,1,2,2,2-pentafluoroethyl)-5-(4,4,5,5-tetramethyl-1,3,2-dioxaborolan-2-yl)-4-(trifluoromethyl)pyrazole

Under argon and at −78° C., 3.03 ml (4.85 mmol) of a butyllithium solution (1.6 M in hexane) are added dropwise over 10 min to a solution of 1.00 g (3.73 mmol) of 1-methyl-3-(1,1,2,2,2-pentafluoroethyl)-4-(trifluoromethyl)pyrazole in 20 ml of tetrahydrofuran. After 45 min, a solution of 1.04 g (5.60 mmol) of 2-isopropoxy-4,4,5,5-tetramethyl-1,3-dioxolane in 4 ml of tetrahydrofuran is added dropwise over 10 min. After 1.5 h, the mixture is allowed to warm to room temperature over a period of 1 h, 0.24 ml (4.12 mmol) of acetic acid is added, the mixture is filtered through Celite, the filter cake is washed with ethyl acetate and the filtrate is concentrated under reduced pressure on a rotary evaporator.

This gives 1.44 g of 1-methyl-3-(1,1,2,2,2-pentafluoroethyl)-5-(4,4,5,5-tetramethyl-1,3,2-dioxaborolan-2-yl)-4-(trifluoromethyl)pyrazole.

¹H-NMR (400 MHz, d₆-DMSO): δ=4.08 (s, 3H); 1.38 (s, 12H).

HPLC-MS: log P ^{a)}=5.05, mass (m/z)=395.0 [M+H]⁺.

### Step 3: Synthesis of 5-(4-bromopyrazol-1-yl)-2-chloro-N-cyclopropylbenzamide

5.00 g (18.2 mmol) of 5-bromo-2-chloro-N-cyclopropylbenzamide, 4.29 g (29.1 mmol) of 4-bromo-1H-pyrazole, 347 mg (1.82 mmol) of copper(I) iodide, 518 mg (3.64 mmol) of trans-1,2-diaminocyclohexane, 907 mg (5.46 mmol) of potassium iodide and 7.55 g (54.6 mmol) of potassium carbonate in 50 ml of dioxane are stirred at 110° C. for 24 h. The reaction mixture is filtered off through Tonsil, the filter cake is washed with ethyl acetate and the filtrate is concentrated under reduced pressure on a rotary evaporator. Further purification is carried out by chromatography on silica gel (mobile phase cyclohexane/ethyl acetate).

This gives 3.0 g of 5-(4-bromopyrazol-1-yl)-2-chloro-N-cyclopropylbenzamide.

¹H-NMR (400 MHz, d₆-DMSO): δ=8.90 (m, 1H); 8.60 (m, 1H); 7.90 (m, 3H); 7.64 (m, 1H); 2.82 (m, 1H); 0.70 (m, 2H); 0.55 (m, 2H).

HPLC-MS: log P ^{a)}=2.36, mass (m/z)=340.0/342.0 [M+H]⁺.

### Step 4: Synthesis of N-{2-fluoro-5-[2′-methyl-5′-(pentafluoroethyl)-4′-(trifluoromethyl)-2′H-1,3′-bipyrazol-4-yl]benzyl}propanamide (Ic-1)

Under argon, 125 mg (0.31 mmol) of 1-methyl-3-(1,1,2,2,2-pentafluoroethyl)-5-(4,4,5,5-tetramethyl-1,3,2-dioxaborolan-2-yl)-4-(trifluoromethyl)pyrazole and 98 mg (0.28 mmol) of 5-(4-bromopyrazol-1-yl)-2-chloro-N-cyclopropylbenzamide are dissolved in 10 ml of dioxane, 12.1 mg (0.14 mmol) of potassium formate, 184 mg (0.86 mmol) of potassium phosphate and 23.5 mg (0.02 mmol) of 1,1′-bis(diphenylphosphino)ferrocenepalladium(II) chloride are added and the mixture is stirred at 90° C. for 7 h. The reaction mixture is filtered off through Tonsil, the filter cake is washed with ethyl acetate and the filtrate is concentrated under reduced pressure on a rotary evaporator. Further purification is by chromatography on silica gel (mobile phase cyclohexane/ethyl acetate) and then on silica gel RP-18 (acetonitrile, HCOOH, water).

This gives 40 mg of N-{2-fluoro-5-[2′-methyl-5′-(pentafluoroethyl)-4′-(trifluoromethyl)-2′H-1,3′-bipyrazol-4-yl]benzyl}propanamide.

¹H-NMR (400 MHz, d₆-DMSO): δ=9.05 (m, 1H); 8.60 (m, 1H); 8.10 (m, 1H); 7.95 (m, 2H); 7.68 (m, 1H); 3.90 (s, 3H); 2.84 (m, 1H); 0.70 (m, 2H); 0.55 (m, 2H).

HPLC-MS: log P ^{a)}=3.83, mass (m/z)=528.0 [M+H]⁺.

### Preparation of 2-chloro-N-cyclopropyl-5-{4-[1-methyl-3-(pentafluoroethyl)-4-(trifluoromethyl)-1H-pyrazol-5-yl]-1H-1,2,3-triazol-1-yl}benzamide (Ig-1)

Reaction Scheme 7 shows the synthesis of the compound Ig-1 according to the invention.

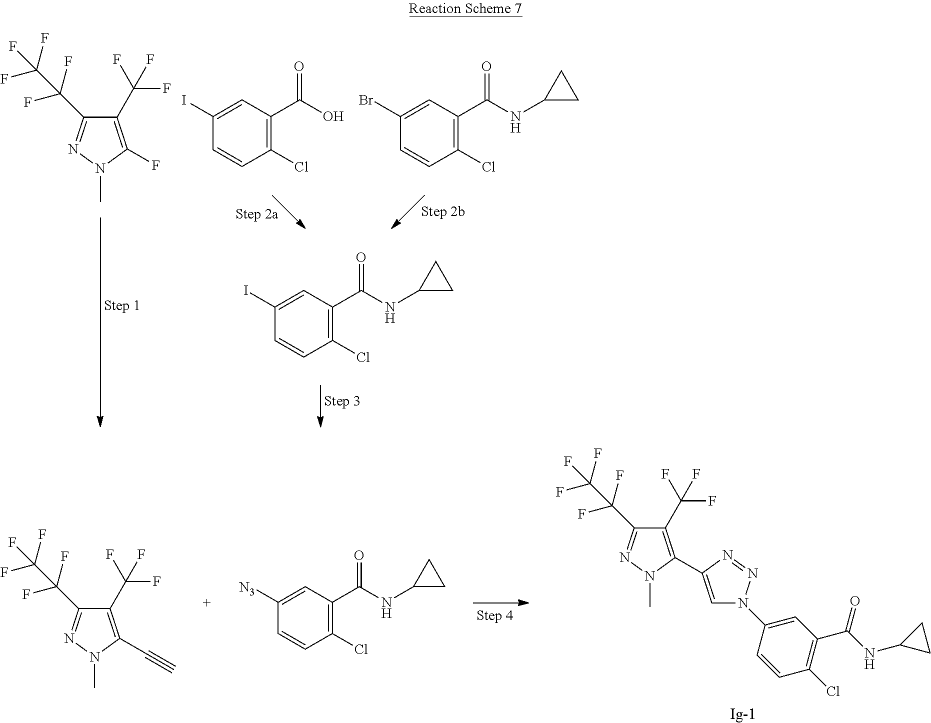

### Step 1: Synthesis of 5-ethynyl-1-methyl-3-(pentafluoroethyl)-4-(trifluoromethyl)-1H-pyrazole

Under an atmosphere of argon and at −78° C., 3.6 ml (5.73 mmol) of an n-butyllithium solution (1.6 M in hexane) are added dropwise to a solution of 0.75 g (7.64 mmol) of (trimethylsilyl)acetylene in THF. After 5 min at −78° C., 1.09 g (3.82 mmol) of 5-fluoro-1-methyl-3-(pentafluoroethyl)-4-(trifluoromethyl)-1H-pyrazole are added dropwise. While stirring, the reaction mixture is warmed from −78° C. to room temperature over a period of 90 min and then cooled to −78° C., and water is added. After warming to room temperature, the reaction mixture is extracted with dichloromethane, and the combined organic phases are dried over magnesium sulphate, filtered and concentrated on a rotary evaporator under reduced pressure.

This gives 936 mg of a black oil which, in addition to the target product 5-ethynyl-1-methyl-3-(pentafluoroethyl)-4-(trifluoromethyl)-1H-pyrazole, also contains the starting material 5-fluoro-1-methyl-3-(pentafluoroethyl)-4-(trifluoromethyl)-1H-pyrazole and traces of 1-methyl-3-(pentafluoroethyl)-4-(trifluoromethyl)-5-[(trimethylsilyl)ethynyl]-1H-pyrazole and 5,5′-ethyne-1,2-diylbis[1-methyl-3-(pentafluoroethyl)-4-(trifluoromethyl)-1H-pyrazole], and which is reacted further in Step 4 without further work-up.

¹H-NMR (400 MHz, d₆-DMSO): δ=5.47 (s, 1H), 4.03 (s, 3H)

HPLC-MS: log P ^{a)}=3.74.

GC-MS: mass (m/z)=292.0 [M]⁺.

### Step 2a (Alternative A): Synthesis of 2-chloro-N-cyclopropyl-5-iodobenzamide

9.39 g (74.0 mmol) of oxalyl chloride and a few drops of N,N-dimethylformamide are added to a suspension of 19.0 g (67.3 mmol) of 2-chloro-5-iodobenzoic acid in dichloromethane (215 ml). The reaction mixture is stirred at room temperature for 30 min and at 35° C. for 30 min and then concentrated on a rotary evaporator under reduced pressure. The residue is dissolved in dry dichloromethane (220 ml) and cooled to 0° C. under an atmosphere of argon, and a solution of 4.23 g (74.0 mmol) of cyclopropylamine in dichloromethane (16 ml) is added dropwise. After 10 min, 10.0 g (77.4 mmol) of N,N-diisopropylethylamine are added at 0° C. The reaction mixture is stirred at room temperature overnight and then diluted with dichloromethane and washed successively with dilute hydrochloric acid (1N), saturated aqueous sodium bicarbonate solution and saturated aqueous sodium chloride solution. The insoluble beige solid (10.9 g of 2-chloro-N-cyclopropyl-5-iodobenzamide) is filtered off and dried. The organic phase is dried over magnesium sulphate, filtered and concentrated on a rotary evaporator under reduced pressure. This gives a further 8.46 g of 2-chloro-N-cyclopropyl-5-iodobenzamide as a colourless solid.

¹H-NMR (400 MHz, d₆-DMSO): δ=8.53 (d, 1H), 7.80-7.75 (m, 1H), 7.71 (d, 1H), 7.28 (d, 1H), 2.82-2.75 (m, 1H), 0.71-0.65 (m, 2H), 0.57-0.49 (m, 2H)

HPLC-MS: log P ^{a)}=2.13; mass (m/z)=322.0 (1C1) [M+H]⁺.

GC-MS: mass (m/z)=321.0 (1C1) [M]⁺.

### Step 2b (Alternative B): Synthesis of 2-chloro-N-cyclopropyl-5-iodobenzamide

761 mg (4.00 mmol) of copper(I) iodide, 569 mg (4.00 mmol) of trans-N,N′-dimethylcyclohexane-1,2-diamine (racemic) and 15.0 g (100 mmol) of sodium iodide are added to a solution of 5.49 g (20.0 mmol) of 5-bromo-2-chloro-N-cyclopropylbenzamide (see, for example, WO2006129237A2) in dioxane (100 ml). The reaction mixture is heated at reflux overnight, then cooled to room temperature and filtered through silica gel using ethyl acetate. The filtrate is concentrated on a rotary evaporator under reduced pressure and purified by column chromatography (SiO₂, cyclohexane/ethyl acetate).

This gives 4.59 g of 2-chloro-N-cyclopropyl-5-iodobenzamide as a slightly yellow solid (the analytical data agree with the target product obtained in Step 2a, see above).

### Step 3: Synthesis of 5-azido-2-chloro-N-cyclopropylbenzamide

128 mg of trans-N,N′-dimethylcyclohexane-1,2-diamine (racemic) are added to a solution of 965 mg (3.00 mmol) of 2-chloro-N-cyclopropyl-5-iodobenzamide, 114 mg (0.60 mmol) of copper(I) iodide, 59.4 mg (0.30 mmol) of sodium ascorbate and 390 mg (6.00 mmol) of sodium azide in dimethyl sulphoxide (16 ml) and water (4 ml). The reaction mixture is stirred at room temperature overnight, ethyl acetate and saturated aqueous sodium chloride solution are then added and the mixture is subsequently extracted with ethyl acetate. The combined organic phases are dried over magnesium sulphate, filtered and concentrated on a rotary evaporator under reduced pressure. The residue is purified by column chromatography (SiO₂, cyclohexane/ethyl acetate).

This gives 503 mg of 5-azido-2-chloro-N-cyclopropylbenzamide as a yellow solid.

¹H-NMR (400 MHz, d₆-DMSO): δ=8.51 (d, 1H), 7.50 (d, 1H), 7.20-7.17 (m, 1H), 7.14 (d, 1H), 2.83-2.67 (m, 1H), 0.71-0.63 (m, 2H), 0.59-0.51 (m, 2H)

HPLC-MS: log P ^{a)}=1.80; mass (m/z)=237.0 (1C1) [M+H]⁺.

GC-MS: mass (m/z)=236.0 (1C1) [M]⁺.

### Step 4: Synthesis of 2-chloro-N-cyclopropyl-5-{4-[1-methyl-3-(pentafluoroethyl)-4-(trifluoromethyl)-1H-pyrazol-5-yl]-1H-1,2,3-triazol-1-yl}benzamide (Ig-1)

17.2 mg (0.09 mmol) of sodium ascorbate and 1.4 mg (0.009 mmol) of copper(II) sulphate are added to a solution of 205 mg (0.87 mmol) of 5-azido-2-chloro-N-cyclopropylbenzamide and 464 mg of the crude product from Step 1, which contains about 60% 5-ethynyl-1-methyl-3-(pentafluoroethyl)-4-(trifluoromethyl)-1H-pyrazole (0.95 mmol) in 14 ml of a mixture of ethanol, water and toluene (about 7:3:1). The reaction mixture is stirred at room temperature overnight. Another 17.2 mg (0.09 mmol) of sodium ascorbate and 1.4 mg (0.009 mmol) of copper(II) sulphate are added and the reaction mixture is stirred at room temperature overnight and then concentrated on a rotary evaporator under reduced pressure. The residue is purified by column chromatography (SiO₂, cyclohexane/ethyl acetate).

This gives 320 mg of 2-chloro-N-cyclopropyl-5-{4-[1-methyl-3-(pentafluoroethyl)-4-(trifluoromethyl)-1H-pyrazol-5-yl]-1H-1,2,3-triazol-1-yl}benzamide as a light-brown solid.

¹H-NMR (400 MHz, d₆-DMSO): δ=9.43 (s, 1H), 8.69 (d, 1H), 8.11-8.07 (m, 2H), 7.80 (d, 1H), 4.00 (s, 3H), 2.88-2.83 (m, 1H), 0.76-0.71 (m, 2H), 0.58-0.51 (m, 2H)

HPLC-MS: log P ^{a)}=3.67; mass (m/z)=529.0 (1C1) [M+H]⁺.

GC-MS: mass (m/z)=528.0 (1C1) [M]⁺.

### Preparation of 2-chloro-N-cyclopropyl-5-{4-[1-methyl-3-(pentafluoroethyl)-4-(trifluoromethyl)-1H-pyrazol-5-yl]-2H-1,2,3-triazol-2-yl}benzamide (If-1)

Reaction Scheme 8 shows the synthesis of the compound If-1 according to the invention.

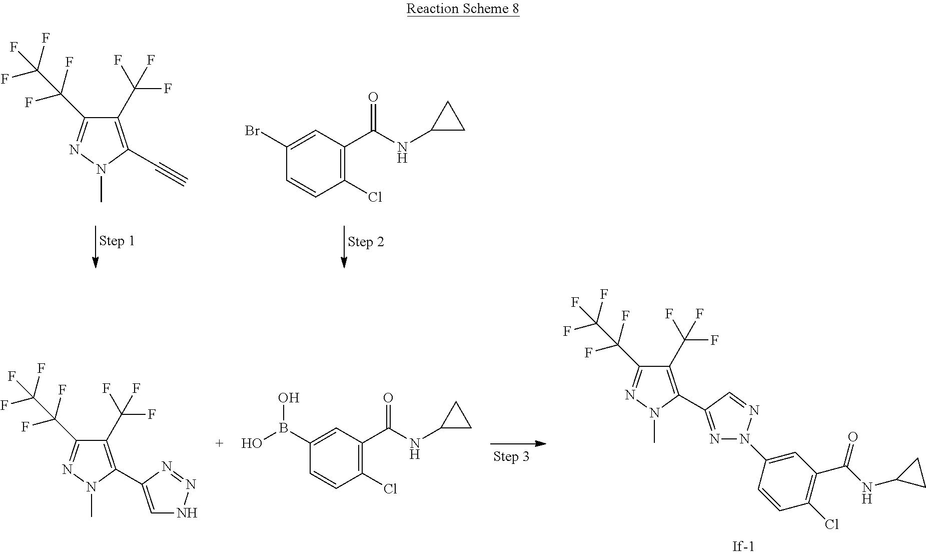 [SMILES: C#Cc1c(C(F)(F)F)c(C(F)(F)C(F)(F)F)nn1C ; O=C(NC1CC1)c1cc(Br)ccc1Cl ; Cn1nc(C(F)(F)C(F)(F)F)c(C(F)(F)F)c1-c1c[nH]nn1 ; O=C(NC1CC1)c1cc(B(O)O)ccc1Cl ; Cn1nc(C(F)(F)C(F)(F)F)c(C(F)(F)F)c1-c1cnn(-c2ccc(Cl)c(C(=O)NC3CC3)c2)n1]

### Step 1: Synthesis of 4-[1-methyl-3-(pentafluoroethyl)-4-(trifluoromethyl)-1H-pyrazol-5-yl]-1H-1,2,3-triazole

After 15 minutes of stirring, 92.9 mg (1.43 mmol) of sodium azide and 464 mg of the crude product from Step 1 of the synthesis of 2-chloro-N-cyclopropyl-5-{4-[1-methyl-3-(pentafluoroethyl)-4-(trifluoromethyl)-1H-pyrazol-5-yl]-1H-1,2,3-triazol-1-yl}benzamide, which contains about 60% 5-ethynyl-1-methyl-3-(pentafluoroethyl)-4-(trifluoromethyl)-1H-pyrazole (0.95 mmol), are added to a solution of 0.71 ml (9.53 mmol) of a 37% strength aqueous formaldehyde solution and 0.08 ml (1.43 mmol) of acetic acid in dioxane. After a further 10 min, a solution of 37.7 mg (0.19 mmol) of sodium ascorbate and 7.6 mg (0.05 mmol) of copper(II) sulphate in water is added. The reaction mixture is stirred at room temperature overnight, water is then added and the mixture is acidified and extracted with dichloromethane. The residue is stirred in aqueous 2M sodium hydroxide solution and then acidified and extracted with dichloromethane. The combined organic phases are dried over magnesium sulphate, filtered and concentrated on a rotary evaporator under reduced pressure.

This gives 106 mg of a brown oil which contains about 62% 4-[1-methyl-3-(pentafluoroethyl)-4-(trifluoromethyl)-1H-pyrazol-5-yl]-1H-1,2,3-triazole and is used without further purification in Step 3.

¹H-NMR (400 MHz, d₆-DMSO): δ=15.90 (broad, 1H), 8.40 (broad, 1H), 3.90 (s, 3H)

HPLC-MS: log P ^{a)}=2.78; mass (m/z)=336.0 [M+H]⁺.

GC-MS: mass (m/z)=335.0 [M]⁺.

### Step 2: Synthesis of [4-chloro-3-(cyclopropylcarbamoyl)phenyl]boronic acid

Under an atmosphere of argon and at −78° C., 100 ml (170 mmol) of a tert-butyllithium solution (1.7M in pentane) are slowly added dropwise to a solution of 15.1 g (54.8 mmol) of 5-bromo-2-chloro-N-cyclopropylbenzamide (see, for example, WO2006129237A2) in dry THF such that the temperature does not exceed −65° C. After the addition is complete, the mixture is stirred at −78° C. for 10 min, 51.6 g (274 mmol) of triisopropyl borate are added and the mixture is then stirred at −78° C. to −60° C. for 3 h. After cooling to −78° C., saturated aqueous ammonium chloride solution is added and the mixture is warmed to room temperature overnight. The reaction mixture is diluted with saturated aqueous ammonium chloride solution and extracted with ethyl acetate. The combined organic phases are washed with water and saturated aqueous sodium chloride solution, dried over magnesium sulphate, filtered and concentrated on a rotary evaporator under reduced pressure. The residue is purified by column chromatography (SiO₂, dichloromethane/methanol with 1% formic acid).

This gives 4.64 g of [4-chloro-3-(cyclopropylcarbamoyl)phenyl]boronic acid as a beige solid.

¹H-NMR (400 MHz, d₆-DMSO): δ=8.44 (d, 1H), 8.25 (s, 2H), 7.79 (d, 1H), 7.77 (s, 1H), 7.43 (d, 1H), 2.86-2.78 (m, 1H), 0.72-0.65 (m, 2H), 0.53-0.49 (m, 2H)

HPLC-MS: log P ^{a)}=0.96; mass (m/z)=240.0 [M+H]⁺.

### Step 3: Synthesis of 2-chloro-N-cyclopropyl-5-{4-[1-methyl-3-(pentafluoroethyl)-4-(trifluoromethyl)-1H-pyrazol-5-yl]-2H-1,2,3-triazol-2-yl}benzamide (If-1)

81.0 mg (0.45 mmol) of copper(II) acetate, 47 mg of pyridine (0.60 mmol) and a spatula of activated 3A molecular sieve are added to a solution of 100 mg (0.30 mmol) of 4-[1-methyl-3-(pentafluoroethyl)-4-(trifluoromethyl)-1H-pyrazol-5-yl]-1H-1,2,3-triazole and 143 mg (0.60 mmol) of [4-chloro-3-(cyclopropylcarbamoyl)phenyl]boronic acid in dry dichloromethane. The reaction mixture is stirred at room temperature for 4 d and then diluted with dichloromethane, adsorbed on kieselguhr and purified by column chromatography (SiO₂, cyclohexane/ethyl acetate) and by preparative HPLC (Phenomenex Gemini 5 micron C18, water/acetonitrile/formic acid).

This gives 10 mg of 2-chloro-N-cyclopropyl-5-{4-[1-methyl-3-(pentafluoroethyl)-4-(trifluoromethyl)-1H-pyrazol-5-yl]-2H-1,2,3-triazol-2-yl}benzamide as a colourless solid.

¹H-NMR (400 MHz, d₆-DMSO): δ=8.69 (d, 1H), 8.61 (s, 1H), 8.14-8.12 (m, 1H), 8.05 (d, 1H), 7.76 (d, 1H), 4.01 (s, 3H), 2.87-2.82 (m, 1H), 0.75-0.70 (m, 2H), 0.57-0.53 (m, 2H)

HPLC-MS: log P ^{a)}=4.17; mass (m/z)=529.0 (1C1) [M+H]⁺.

GC-MS: mass (m/z)=528.0 (1C1) [M]⁺.

### Preparation of 2-chloro-N-cyclopropyl-5-{5-[1-methyl-3-(pentafluoroethyl)-4-(trifluoromethyl)-1H-pyrazol-5-yl]-2H-tetrazol-2-yl}benzamide (Ih-1)

Reaction Scheme 9 shows the synthesis of the compound Ih-1 according to the invention.

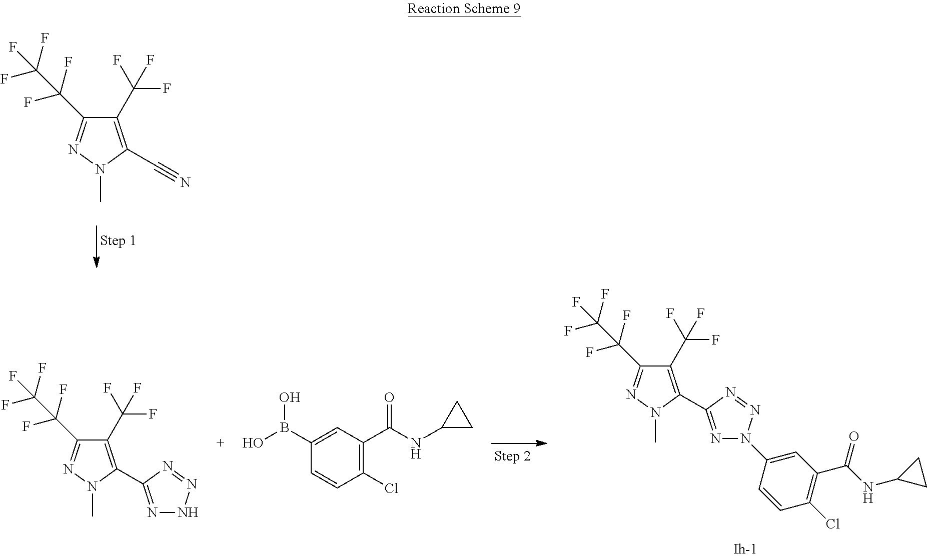 [SMILES: Cn1nc(C(F)(F)C(F)(F)F)c(C(F)(F)F)c1C#N ; Cn1nc(C(F)(F)C(F)(F)F)c(C(F)(F)F)c1-c1nn[nH]n1 ; O=C(NC1CC1)c1cc(B(O)O)ccc1Cl ; Cn1nc(C(F)(F)C(F)(F)F)c(C(F)(F)F)c1-c1nnn(-c2ccc(Cl)c(C(=O)NC3CC3)c2)n1]

### Step 1: Synthesis of 5-[1-methyl-3-(pentafluoroethyl)-4-(trifluoromethyl)-1H-pyrazol-5-yl]-2H-tetrazole

With microwave irradiation (Biotage Initiator), a suspension of 250 mg (0.85 mmol) of 1-methyl-3-(pentafluoroethyl)-4-(trifluoromethyl)-1H-pyrazole-5-carbonitrile, 66.5 mg (1.02 mmol) of sodium azide and 54.8 mg (1.02 mmol) of ammonium chloride in N,N-dimethylformamide (1.0 ml) is stirred at 100° C. for 2 h. After cooling to room temperature, water and 1M hydrochloric acid are added and the reaction mixture is extracted with ethyl acetate. The combined organic phases are dried over magnesium sulphate, filtered and concentrated on a rotary evaporator under reduced pressure. The residue is purified by column chromatography (RP18-SiO₂, water/acetonitrile with 0.05% formic acid).

This gives 176 mg of 5-[1-methyl-3-(pentafluoroethyl)-4-(trifluoromethyl)-1H-pyrazol-5-yl]-2H-tetrazole as a colourless solid.

¹H-NMR (400 MHz, d₆-DMSO): δ=5.90 (broad, 1H), 3.99 (s, 3H)

HPLC-MS: log P ^{a)}=2.11; mass (m/z)=337.0 [M+H]⁺.

### Step 2: Synthesis of 2-chloro-N-cyclopropyl-5-{5-[1-methyl-3-(pentafluoroethyl)-4-(trifluoromethyl)-1H-pyrazol-5-yl]-2H-tetrazol-2-yl}benzamide (Ih-1)

122 mg (0.67 mmol) of copper(II) acetate, 71 mg of pyridine (0.89 mmol) and a spatula of activated 3A molecular sieve are added to a solution of 150 mg (0.45 mmol) of 5-[1-methyl-3-(pentafluoroethyl)-4-(trifluoromethyl)-1H-pyrazol-5-yl]-2H-tetrazole and 214 mg (0.89 mmol) of [4-chloro-3-(cyclopropylcarbamoyl)phenyl]boronic acid in dry dichloromethane. The reaction mixture is stirred at room temperature for 4 d and then diluted with dichloromethane, adsorbed on kieselguhr and purified by column chromatography (SiO₂, cyclohexane/ethyl acetate) and by preparative HPLC (Phenomenex Gemini 5 micron C18, water/acetonitrile/formic acid).

This gives 4 mg of 2-chloro-N-cyclopropyl-5-{5-[1-methyl-3-(pentafluoroethyl)-4-(trifluoromethyl)-1H-pyrazol-5-yl]-2H-tetrazol-2-yl}benzamide as a colourless solid.

¹H-NMR (400 MHz, d₆-DMSO): δ=8.76 (d, 1H), 8.25-8.22 (m, 1H), 8.17 (d, 1H), 7.88 (d, 1H), 4.15 (s, 3H), 2.90-2.82 (m, 1H), 0.78-0.71 (m, 2H), 0.60-0.50 (m, 2H)

HPLC-MS: log P ^{a)}=4.06; mass (m/z)=530.0 (1C1) [M+H]⁺.

### Preparation of 2-chloro-N-cyclopropyl-5-[2′-methyl-5′-(pentafluoroethyl)-4′-(trifluoromethyl)-1H,2′H-3,3′-bipyrazol-1-yl]benzamide (Id-1)

Reaction Scheme 10 shows the synthesis of the compound Id-1 according to the invention.

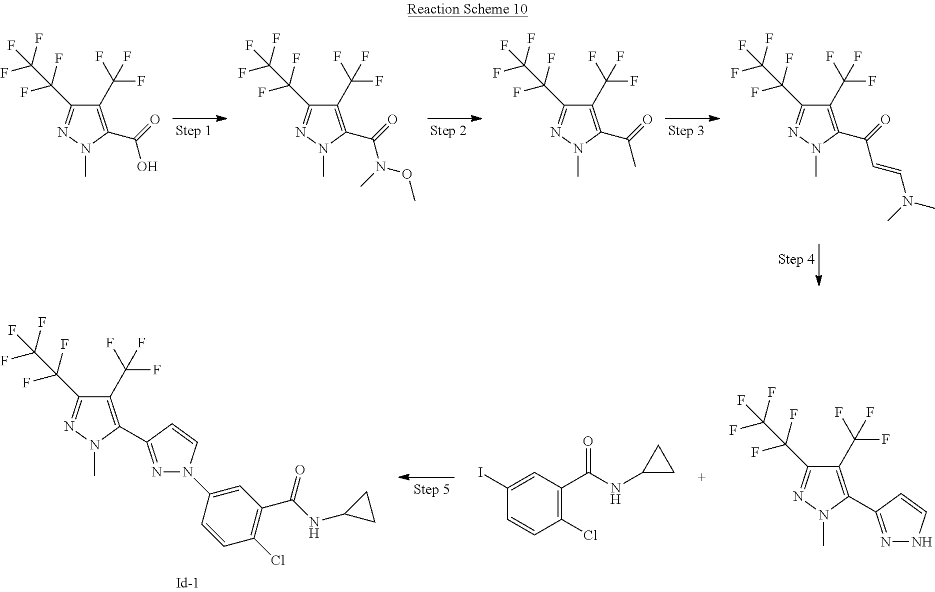

### Step 1: Synthesis of N-methoxy-N,1-dimethyl-3-(pentafluoroethyl)-4-(trifluoromethyl)-1H-pyrazole-5-carboxamide

Five drops of N,N-dimethylformamide and 42.8 ml (481 mmol) of oxalyl chloride are added to a solution of 50.0 g (160 mmol) of 1-methyl-3-(pentafluoroethyl)-4-(trifluoromethyl)-1H-pyrazole-5-carboxylic acid in dichloromethane (250 ml). The reaction mixture is heated at reflux for 90 min and then cooled and concentrated on a rotary evaporator under reduced pressure. The residue is dissolved in dichloromethane (250 ml) and added to a solution of 18.8 g (192 mmol) of N, O-dimethylhydroxylamine hydrochloride in dichloromethane (250 ml), and 55.8 ml (400 mmol) of triethylamine are then added dropwise. The reaction mixture is stirred at room temperature overnight and then diluted with dichloromethane and stirred into 1N hydrochloric acid. The mixture is extracted with dichloromethane. The combined organic phases are washed with 1N aqueous sodium hydroxide solution and saturated aqueous sodium chloride solution, dried over magnesium sulphate, filtered and concentrated on a rotary evaporator under reduced pressure.

This gives 53.2 g of N-methoxy-N,1-dimethyl-3-(pentafluoroethyl)-4-(trifluoromethyl)-1H-pyrazole-5-carboxamide as an orange oil.

¹H-NMR (400 MHz, d₆-DMSO): δ=3.97 (s, 3H), 3.54 (s, 3H), 3.37 (s, 3H)

HPLC-MS: log P ^{a)}=3.22; mass (m/z)=356.0 [M+H]⁺.

GC-MS: mass (m/z)=355.0 [M]⁺.

### Step 2: Synthesis of 1-[1-methyl-3-(pentafluoroethyl)-4-(trifluoromethyl)-1H-pyrazol-5-yl]ethanone

Under an atmosphere of argon and at −5° C., 150 ml (449 mmol) of a methylmagnesium bromide solution (3M in ether) are slowly added dropwise to a solution of 53.2 g (150 mmol) of N-methoxy-N,1-dimethyl-3-(pentafluoroethyl)-4-(trifluoromethyl)-1H-pyrazole-5-carboxamide in dry THF (400 ml) such that the temperature does not exceed 0° C. After the complete addition, the mixture is initially warmed to room temperature and then stirred at 50° C. overnight. The reaction mixture is cooled to room temperature, water and saturated aqueous ammonium carbonate solution are added and the mixture is extracted with tert-butyl methyl ether. The combined organic phases are washed with water and saturated aqueous sodium chloride solution, dried over magnesium sulphate, filtered and concentrated on a rotary evaporator under reduced pressure. The residue is purified by distillation (0.18 mbar, 52° C.).

This gives 40.2 g of 1-[1-methyl-3-(pentafluoroethyl)-4-(trifluoromethyl)-1H-pyrazol-5-yl]ethanone as a colourless solid.

¹H-NMR (400 MHz, d₆-DMSO): δ=4.01 (s, 3H), 2.66 (s, 3H)

HPLC-MS: log P ^{a)}=3.61;

GC-MS: mass (m/z)=310.0 [M]⁺.

### Step 3: Synthesis of (2E)-3-(dimethylamino)-1-[1-methyl-3-(pentafluoroethyl)-4-(trifluoromethyl)-1H-pyrazol-5-yl]prop-2-en-1-one

A solution of 1.50 g (4.84 mmol) of 1-[1-methyl-3-(pentafluoroethyl)-4-(trifluoromethyl)-1H-pyrazol-5-yl]ethanone in 3.2 ml (24.2 mmol) of N,N-dimethylformamide dimethyl acetal is heated at reflux overnight. The reaction mixture is concentrated on a rotary evaporator under reduced pressure and dried under high vacuum for 30 min.

This gives 1.86 g of (2E)-3-(dimethylamino)-1-[1-methyl-3-(pentafluoroethyl)-4-(trifluoromethyl)-1H-pyrazol-5-yl]prop-2-en-1-one as an orange-brown oil which are used without further purification in Step 4.

HPLC-MS: log P ^{a)}=2.77; mass (m/z)=366.0 [M+H]⁺.

GC-MS: mass (m/z)=365.0 [M]⁺.

### Step 4: Synthesis of 2′-methyl-5′-(pentafluoroethyl)-4′-(trifluoromethyl)-1H,2′H-3,3′-bipyrazole

0.55 ml (11.3 mmol) of hydrazine hydrate are added to a solution of 1.38 g (3.77 mmol) of (2E)-3-(dimethylamino)-1-[1-methyl-3-(pentafluoroethyl)-4-(trifluoromethyl)-1H-pyrazol-5-yl]prop-2-en-1-one in ethanol (25 ml). The reaction mixture is heated at reflux for 1 h and then cooled and concentrated on a rotary evaporator under reduced pressure and dried under high vacuum for 20 min. The residue is purified by column chromatography (RP18-SiO₂, water/acetonitrile with 0.05% formic acid).

This gives 984 mg of 2′-methyl-5′-(pentafluoroethyl)-4′-(trifluoromethyl)-1H,2′H-3,3′-bipyrazole as a yellow solid.

¹H-NMR (400 MHz, d₆-DMSO): δ=13.59 (s, 1H), 8.02 (s, 1H), 6.66 (s, 1H), 3.90 (s, 3H)

HPLC-MS: log P ^{a)}=3.03; mass (m/z)=334.0 [M+H]⁺.

GC-MS: mass (m/z)=333.0 [M]⁺.

### Step 5: Synthesis of 2-chloro-N-cyclopropyl-5-[2′-methyl-5′-(pentafluoroethyl)-4′-(trifluoromethyl)-1H,2′H-3,3′-bipyrazol-1-yl]benzamide (Id-1)

A solution of 100 mg (0.30 mmol) of 2′-methyl-5′-(pentafluoroethyl)-4′-(trifluoromethyl)-1H,2′H-3,3′-bipyrazole, 115 mg (0.36 mmol) of 2-chloro-N-cyclopropyl-5-iodobenzamide, 5.4 mg (0.03 mmol) of copper(II) acetate and 195 mg (0.60 mmol) of caesium carbonate in degassed N,N-dimethylformamide is stirred in a closed crimp vial at 110° C. overnight. After cooling to room temperature, the reaction mixture is diluted with ethyl acetate and water and extracted with ethyl acetate. The combined organic phases are washed with water and saturated aqueous sodium chloride solution, dried over magnesium sulphate, filtered and concentrated on a rotary evaporator under reduced pressure. The residue is purified by column chromatography (SiO₂, cyclohexane/ethyl acetate) and by preparative HPLC (Phenomenex Gemini 5 micron C18, water/acetonitrile/formic acid).

This gives 25 mg of 2-chloro-N-cyclopropyl-5-[2′-methyl-5′-(pentafluoroethyl)-4′-(trifluoromethyl)-1H,2′H-3,3′-bipyrazol-1-yl]benzamide as a colourless solid.

¹H-NMR (400 MHz, d₆-DMSO): δ=8.86 (d, 1H), 8.62 (d, 1H), 8.00-7.96 (m, 2H), 7.68 (d, 1H), 7.00 (d, 1H), 3.97 (s, 3H), 2.87-2.82 (m, 1H), 0.74-0.67 (m, 2H), 0.57-0.54 (m, 2H)

HPLC-MS: log P ^{a)}=4.05; mass (m/z)=528.0 (1C1) [M+H]⁺.

GC-MS: mass (m/z)=527.0 (1C1) [M]⁺.

^{a)} Unless indicated otherwise, the following method was used to determine the log P values and masses: The determination of the given log P values was carried out in accordance with EEC Directive 79/831 Annex V.A8 by HPLC (High Performance Liquid Chromatography) on a reversed-phase column (C18). Agilent 1100 LC system; 50*4.6 Zorbax Eclipse Plus C18 1.8 micron; mobile phase A: acetonitrile (0.1% formic acid); mobile phase B: water (0.09% formic acid); linear gradient from 10% acetonitrile to 95% acetonitrile in 4.25 min, then 95% acetonitrile for a further 1.25 min; oven temperature 55° C.; flow rate: 2.0 ml/min. Mass detection is effected by means of an Agilend MSD system.

The stated mass is the peak of the isotope pattern of the [M+H]⁺ ion of the highest intensity; if the [M−H]⁻ ion was detected, the stated mass is marked with ².

² The stated mass is the peak of the isotope pattern of the [M−H]⁻ ion of the highest intensity.

### Biological Working Examples for Applications in the Animal Health Sector and in Crop Protection

Ctenocephalides felis—In Vitro Contact Test

For the coating of the test tubes, 9 mg of active compound are first dissolved in 1 ml of acetone p.a. and then diluted to the desired concentration with acetone p.a. 250 μl of the solution are distributed homogeneously on the inner walls and the base of a 25 ml test tube by turning and rocking on an orbital shaker (rocking rotation at 30 rpm for 2 h). With 900 ppm active compound solution and internal surface 44.7 cm², given homogeneous distribution, an area-based dose of 5 μg/cm² is achieved.

After the solvent has evaporated off, the tubes are populated with 5-10 adult cat fleas (Ctenocephalides felis), sealed with a perforated plastic lid and incubated in a horizontal position at room temperature and ambient humidity. After 48 h, efficacy is determined. To this end, the test tubes are stood upright and the fleas are knocked to the base of the tube. Fleas which remain motionless at the base or move in an uncoordinated manner are considered to be dead or moribund.

A substance shows good efficacy against Ctenocephalides felis if at least 80% efficacy was achieved in this test at an application rate of 5 μg/cm². 100% efficacy means that all the fleas were dead or moribund. 0% efficacy means that no fleas were harmed.

In this test, for example, the following compounds from the preparation examples show an efficacy of 100% at an application rate of 5 μg/cm²: Ic-1, Ig-1

Rhipicephalus sanguineus—In Vitro Contact Tests

For the coating of the test tubes, 9 mg of active compound are first dissolved in 1 ml of acetone p.a. and then diluted to the desired concentration with acetone p.a. 250 μl of the solution are distributed homogeneously on the inner walls and the base of a 25 ml test tube by turning and rocking on an orbital shaker (rocking rotation at 30 rpm for 2 h). With 900 ppm active compound solution and internal surface 44.7 cm², given homogeneous distribution, an area-based dose of 5 μg/cm² is achieved.

After the solvent has evaporated off, the tubes are populated with 5-10 adult dog ticks (Rhipicephalus sanguineus), sealed with a perforated plastic lid and incubated in a horizontal position in the dark at room temperature and ambient humidity. After 48 h, efficacy is determined. To this end, the ticks are knocked to the floor of the tube and incubated on a hotplate at 45-50° C. for not more than 5 min. Ticks which remain motionless on the floor or move in such an uncoordinated manner that they are unable to deliberately avoid the heat by climbing upwards are considered to be dead or moribund.

A substance shows good efficacy against Rhipicephalus sanguineus if at least 80% efficacy was achieved in this test at an application rate of 5 μg/cm². An efficacy of 100% means that all the ticks were dead or moribund. 0% efficacy means that none of the ticks had been harmed.

In this test, for example, the following compounds from the preparation examples show an efficacy of 100% at an application rate of 5 μg/cm²: Ic-1, Ig-1

Amblyomma hebaraeum Test (AMBYHE)
Solvent: dimethyl sulphoxide

To produce a suitable preparation of active compound, 10 mg of active compound are mixed with 0.5 ml of dimethyl sulphoxide, and the concentrate is diluted with water to the desired concentration.

Tick nymphs (Amblyomma hebraeum) are placed into perforated plastic beakers and immersed in the desired concentration for one minute. The ticks are transferred on filter paper into a Petri dish and stored in a climate-controlled cabinet.

After 42 days, the kill in % is determined. 100% means that all of the ticks have been killed; 0% means that none of the ticks have been killed.

In this test, for example, the following compounds of the preparation examples show an efficacy of 100% at an application rate of 20 ppm: Ic-1, Ig-1

Boophilus microplus—Dip Test (BOOPMI Dip)
Test animals: cattle ticks (Boophilus microplus) Parkhurst strain, SP-resistant
Solvent: dimethyl sulphoxide

10 mg of active compound are dissolved in 0.5 ml of dimethyl sulphoxide. To produce a suitable formulation, the active compound solution is diluted with water to the concentration desired in each case.

This active compound preparation is pipetted into tubes. 8-10 engorged adult female cattle ticks (Boophilus microplus) are transferred into a further tube with holes. The tube is immersed into the active compound preparation, and all the ticks are completely wetted. After the liquid has run out, the ticks are transferred on filter discs into plastic dishes and stored in a climate-controlled room.

Efficacy is assessed after 7 days by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples show an efficacy of 100% at an application rate of 20 ppm: Ic-1, Ig-1

Boophilus microplus—Injection Test (BOOPMI Inj)
Solvent: dimethyl sulphoxide

To produce a suitable preparation of active compound, 10 mg of active compound are mixed with 0.5 ml of solvent and the concentrate is diluted with solvent to the desired concentration.

1 μl of the active compound solution is injected into the abdomen of 5 engorged adult female cattle ticks (Boophilus microplus). The animals are transferred into dishes and kept in a climate-controlled room.

The activity is assessed after the desired time by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples show an efficacy of 100% at an application rate of 20 μg/animal: If-1, Ig-1

In this test, for example, the following compounds from the preparation examples show an efficacy of 100% at an application rate of 4 μg/animal: Ic-1

Ctenocephalides felis—Oral Test (CTECFE)
Solvent: dimethyl sulphoxide

To produce a suitable preparation of active compound, 10 mg of active compound are mixed with 0.5 ml of dimethyl sulphoxide. Dilution with citrated cattle blood gives the desired concentration.

About 20 unfed adult cat fleas (Ctenocephalides felis) are placed into a chamber which is closed at the top and bottom with gauze. A metal cylinder whose bottom end is closed with parafilm is placed onto the chamber. The cylinder contains the blood/active compound preparation, which can be imbibed by the fleas through the parafilm membrane.

After 2 days, the kill in % is determined. 100% means that all of the fleas have been killed; 0% means that none of the fleas have been killed.

In this test, for example, the following compounds from the preparation examples show an efficacy of 100% at an application rate of 100 ppm: If-1, Ig-1

In this test, for example, the following compounds from the preparation examples show an efficacy of 100% at an application rate of 20 ppm: Ic-1

Lucilia cuprina-Test (LUCICU)
Solvent: dimethyl sulphoxide

To produce a suitable preparation of active compound, 10 mg of active compound are mixed with 0.5 ml of dimethyl sulphoxide, and the concentrate is diluted with water to the desired concentration.

About 20 L1 larvae of the Australian sheep blowfly (Lucilia cuprina) are transferred into a test vessel containing minced horsemeat and the active compound preparation of the desired concentration.

After 2 days, the kill in % is determined. 100% means that all the larvae have been killed; 0% means that no larvae have been killed.

In this test, for example, the following compounds from the preparation examples show an efficacy of 100% at an application rate of 100 ppm: If-1, Ig-1

In this test, for example, the following compounds from the preparation examples show an efficacy of 100% at an application rate of 20 ppm: Ic-1

Musca domestica-Test (MUSCDO)
Solvent: dimethyl sulphoxide

To produce a suitable preparation of active compound, 10 mg of active compound are mixed with 0.5 ml of dimethyl sulphoxide, and the concentrate is diluted with water to the desired concentration.

Vessels containing a sponge treated with sugar solution and the active compound preparation of the desired concentration are populated with 10 adult houseflies (Musca domestica).

After 2 days, the kill in % is determined. 100% means that all of the flies have been killed; 0% means that none of the flies have been killed.

In this test, for example, the following compounds from the preparation examples show an efficacy of 100% at an application rate of 100 ppm: If-1, Ig-1

In this test, for example, the following compounds from the preparation examples show an efficacy of 100% at an application rate of 20 ppm: Ic-1

Myzus persicae—Spray Test (MYZUPE)
Solvent: 78 parts by weight of acetone
1.5 parts by weight of dimethylformamide
   Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the preparation is diluted with emulsifier-containing water.

Discs of Chinese cabbage leaves (Brassica pekinensis) infested by all stages of the green peach aphid (Myzus persicae) are sprayed with an active compound preparation of the desired concentration.

After 6 days, efficacy in % is determined. 100% means that all the aphids have been killed; 0% means that no aphids have been killed.

In this test, for example, the following compounds from the preparation examples show efficacy of 90% at an application rate of 100 g/ha: Ic-1

Phaedon cochleariae—Spray Test (PHAECO)
Solvent: 78.0 parts by weight of acetone
1.5 parts by weight of dimethylformamide
   Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the preparation is diluted with emulsifier-containing water.

Discs of Chinese cabbage leaves (Brassica pekinensis) are sprayed with an active compound preparation of the desired concentration and, after drying, populated with larvae of the mustard beetle (Phaedon cochleariae).

After 7 days, efficacy in % is determined. 100% means that all the beetle larvae have been killed; 0% means that no beetle larvae have been killed.

In this test, for example, the following compounds from the preparation examples show efficacy of 100% at an application rate of 100 g/ha: Ic-1, Id-1, If-1, Ig-1

In this test, for example, the following compounds from the preparation examples show efficacy of 100% at an application rate of 20 g/ha: Ih-1

Spodoptera frugiperda—Spray Test (SPODFR)

Solvent: 78.0 parts by weight of acetone
1.5 parts by weight of dimethylformamide
   Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the preparation is diluted with emulsifier-containing water.

Leaf discs of maize (Zea mays) are sprayed with an active compound preparation of the desired concentration and, after drying, populated with caterpillars of the armyworm (Spodoptera frugiperda).

After 7 days, efficacy in % is determined. 100% means that all the caterpillars have been killed; 0% means that no caterpillars have been killed.

In this test, for example, the following compounds from the preparation examples show efficacy of 100% at an application rate of 100 g/ha: Ic-1, Id-1, If-1, Ig-1, Ih-1

Tetranychus urticae—Spray Test, OP-Resistant (TETRUR)
Solvent: 78.0 parts by weight of acetone
1.5 parts by weight of dimethylformamide
   Emulsifier: alkylaryl polyglycol ether

To produce a suitable preparation of active compound, 1 part by weight of active compound is dissolved using the specified parts by weight of solvent and made up with water containing an emulsifier concentration of 1000 ppm until the desired concentration is attained. To produce further test concentrations, the preparation is diluted with emulsifier-containing water.

Discs of bean leaves (Phaseolus vulgaris) infested by all stages of the greenhouse red spider mite (Tetranychus urticae) are sprayed with an active ingredient formulation of the desired concentration.

After 6 days, efficacy in % is determined. 100% means that all the spider mites have been killed; 0% means that no spider mites have been killed.

In this test, for example, the following compounds from the preparation examples show an efficacy of 100% at an application rate of 100 g/ha: Ic-1, If-1, Ig-1

In this test, for example, the following compounds from the preparation examples show an efficacy of 90% at an application rate of 100 g/ha: Ih-1

1. Compound of formula (I) 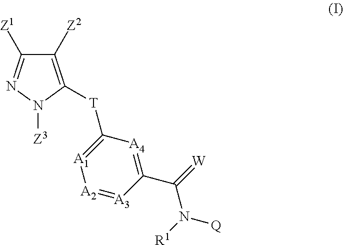
in which
R¹ represents hydrogen or a group, optionally substituted by at least one radical M¹, selected from the group consisting of (C₁-C₆)-alkyl, (C₂-C₆)-alkenyl, (C₂-C₆)-alkynyl, (C₃-C₇)-cycloalkyl, (C₁-C₆)-alkylcarbonyl, (C₁-C₆)-alkoxycarbonyl, aryl-(C₁-C₃)-alkyl and heteroaryl-(C₁-C₃)-alkyl;
the chemical moieties
A₁ represents CR² or nitrogen,
A₂ represents CR³ or nitrogen,
A₃ represents CR⁴ or nitrogen, and
A₄ represents CR⁵ or nitrogen,
where not more than three of the chemical moieties A₁ to A₄ simultaneously represent nitrogen;
R², R³, R⁴ and R⁵ independently of one another represent hydrogen, halogen, cyano, amino, nitro, or a group, optionally substituted by at least one radical M¹, selected from the group consisting of (C₁-C₆)-alkyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkylamino,
if neither of the A₂ and A₃ moieties is nitrogen, R³ and R⁴ together with the carbon atom to which they are bonded may form a 5- or 6-membered ring containing 0, 1 or 2 nitrogen atoms and/or 0 or 1 oxygen atom and/or 0 or 1 sulphur atom, or
if neither of the A₁ and A₂ moieties represents nitrogen, R² and R³ together with the carbon atom to which they are bonded may form a 6-membered ring containing 0, 1 or 2 nitrogen atoms;
W represents oxygen or sulphur;
Q represents hydrogen, hydroxy, or a group, optionally substituted by at least one radical M¹, selected from the group consisting of amino, N—(C₁-C₆)-alkylamino, N—(C₁-C₆)-alkylcarbonylamino, N,N-di-(C₁-C₆)-alkylamino, (C₁-C₆)-alkyl, (C₁-C₆)-alkoxy, (C₂-C₆)-alkenyl, (C₂-C₆)-alkynyl, (C₃-C₆)-cycloalkyl, heterocycloalkyl having 3 to 9 ring atoms, (C₁-C₆)-cycloalkyl-(C₁-C₆)-alkyl, (C₆)-aryl-(C₁-C₆)-alkyl, heteroaryl-(C₁-C₆)-alkyl having 5 to 7 ring atoms; or
Q represents an unsaturated 6-membered carbocycle which is optionally mono- or polysubstituted by V or represents an unsaturated 5- or 6-membered heterocyclic ring which is optionally mono- or polysubstituted by V, where
V independently of one another represent halogen, cyano, amino, nitro or a group, optionally substituted by at least one radical M¹, selected from the group consisting of (C₁-C₆)-alkyl, (C₂-C₄)-alkenyl, (C₂-C₄)-alkynyl, (C₃-C₆)-cycloalkyl, (C₁-C₆)-alkoxy, N—(C₁-C₆)-alkoxyimino-(C₁-C₃)-alkyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkyl)amino;
T represents one of the 5-membered heteroaromatics T1-T8 listed below, where the bond to the pyrazole head group [C₃N₂Z₁Z₂Z₃] is marked with an asterisk *, 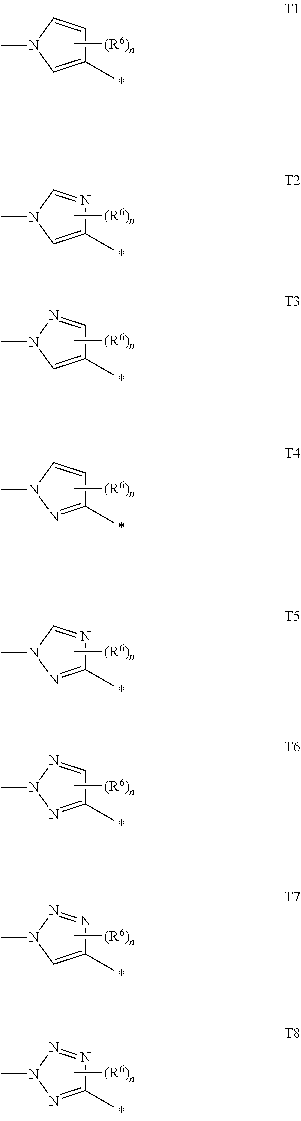
where
R⁶ independently of one another represent halogen, cyano, nitro or a group, optionally substituted by at least one radical M¹, selected from the group consisting of amino, (C₁-C₆)-alkyl, (C₁-C₆)-alkoxy, (C₁-C₆)-alkylcarbonyl, (C₁-C₆)-alkylsulphanyl, (C₁-C₆)-alkylsulphinyl, (C₁-C₆)-alkylsulphonyl, N—(C₁-C₆)-alkylamino and N,N-di-(C₁-C₆)-alkyl)amino;
n represents the values 0-2;
Z¹ represents a group, optionally substituted by at least one radical M¹, selected from the group consisting of (C₁-C₆)-alkyl, (C₁-C₄)-alkoxy, cyano, hydroxycarbonyl, (C₁-C₄)-alkoxycarbonyl, (C₁-C₄)-alkylcarbamoyl, (C₃-C₆)-cycloalkylcarbamoyl, phenyl, preferably represents (C₁-C₆)-alkyl optionally substituted by at least one radical M¹;
Z² represents hydrogen, halogen, cyano, nitro or a group, optionally substituted by at least one radical M¹, selected from the group consisting of (C₁-C₆)-alkyl, (C₁-C₆)-alkylcarbonyl, (C₁-C₆)-alkylsulphanyl, (C₁-C₆)-alkylsulphinyl and (C₁-C₆)-alkylsulphonyl;
Z³ represents hydrogen or a group, optionally substituted by at least one radical M¹, selected from the group consisting of (C₁-C₆)-alkyl, (C₃-C₆)-cycloalkyl, (C₁-C₆)-alkenyl, (C₁-C₆)-alkynyl, (C₆)-aryl and hetaryl having 5 or 6 ring atoms;
M¹ represents halogen, cyano, isocyano, azido, hydroxy, nitro, formyl, carboxyl or a group equivalent to the carboxyl group, or a group, optionally substituted by at least one radical M², selected from the group consisting of amino, (C₁-C₄)-alkyl, (C₁-C₄)-haloalkyl, (C₁-C₄)-alkoxy, (C₁-C₄)-haloalkoxy, (C₁-C₄)-alkoxy-(C₁-C₄)-alkoxy, (C₁-C₄)-alkoxy-(C₁-C₄)-alkyl, (C₁-C₄)-alkylsulphanyl, (C₁-C₄)-haloalkylsulphanyl, (C₁-C₄)-alkoxycarbonyl, (C₁-C₄)-alkylcarbonyl, carbamoyl, mono- and N,N-di-(C₁-C₄)-alkylaminocarbonyl, (C₁-C₄)-acylamino, mono- and N,N-di-(C₁-C₄)-alkylamino, tri-(C₁-C₄)-alkylsilyl, (C₃-C₆)-cycloalkyl, C₆-aryl, heterocyclyl having 3 to 6 ring atoms, where each of the last-mentioned cyclic groups may also be attached via heteroatoms or a divalent functional —CH₂— or —C₂H₄— group, (C₁-C₄)-alkylsulphinyl, where both enantiomers of the (C₁-C₄)-alkylsulphonyl group are included, (C₁-C₄)-alkylsulphonyl, (C₁-C₄)-alkylphosphinyl, (C₁-C₄)-alkylsulphanyl-(C₁-C₄)-alkyl, (C₁-C₄)-alkoxy-(C₁-C₄)-alkyl, mono- and N,N-di-(C₁-C₄)-alkylamino-(C₁-C₄)-alkyl and hydroxy-(C₁-C₄)-alkyl; and
M² represents amino, hydroxy, halogen, nitro, cyano, isocyano, mercapto, isothiocyanato, carboxyl or carboxamide.

2. Compound of formula (I) according to claim 1 in which Z¹ and Z² independently of one another represent perhalogenated C₁-C₄-alkyl and Z³ represents C₁-C₄-alkyl.

3. Compound of formula (I) according to claim 1 in which Z¹ represents perfluorinated ethyl (C₂F₅), Z² represents perfluorinated methyl (CF₃) and Z³ represents methyl.

4. Compound of formula (I) according to claim 1, in which T represents T3 (formula (Ic)), T6 (formula (If)), T7 (formula (Ig)) or T8 (formula (Ih)).

5. Compound of formula (I) according to claim 1, in which Q represents hydrogen, C₁-C₆-alkyl, C₃-C₆-cycloalkyl, C₁-C₆-alkoxy or benzyl.

6. Compound of formula (I) according to claim 1, in which
T represents T3 (formula (Ic)), T6 (formula (If)), T7 (formula (Ig)) or T8 (formula (Ih)) and n in T represents 0;
A₁ represents CR₂, where R₂ represents hydrogen;
A₂ represents CR₃, where R₃ represents hydrogen;
A₃ represents CR₄, where R₄ represents hydrogen, halogen, (C₁-C₄)-alkyl or (C₁-C₄)-alkoxy;
A₄ represents CR₅, where R₅ represents hydrogen;
W represents oxygen;
n in T represents 0;
Z¹ represents halogenated (C₁-C₄)-alkyl;
Z² represents halogenated (C₁-C₄)-alkyl;
Z³ represents (C₁-C₄)-alkyl;
R₁ represents hydrogen or C₁-C₄-alkyl;
Q represents hydrogen or a group, optionally substituted by at least one radical M¹, selected from the group consisting of (C₁-C₆)-alkyl and (C₃-C₆)-cycloalkyl, where M¹ independently of one another represent cyano, chlorine, bromine, iodine or fluorine.

7. Compound of formula (I) according to claim 1, in which
T represents T3 (formula (Ic)), T6 (formula (If)), T7 (formula (Ig)) or T8 (formula (Ih)) and n in T represents 0;
A₁ represents CR₂, where R₂ represents hydrogen;
A₂ represents CR₃, where R₃ represents hydrogen;
A₃ represents CR₄, where R₄ represents chlorine;
A₄ represents CR₅, where R₅ represents hydrogen;
W represents oxygen;
n in T represents 0;
Z¹ represents perfluorinated ethyl;
Z² represents perfluorinated methyl;
Z³ represents methyl;
R¹ represents hydrogen or (C₁-C₄)-alkyl;
Q represents (C₃-C₆)-cycloalkyl, optionally cyclopropyl.

8. A product comprising a compound of formula (I) according to claim 1 for controlling one or more insects, arachnids and/or nematodes.

9. Pharmaceutical composition comprising at least one compound according to claim 1.

10. Compound according to claim 1, for use as a medicament.

11. A product comprising a compound according to claim 1, for producing one or more pharmaceutical compositions for controlling one or more parasites on animals.

12. Process for producing a crop protection composition comprising one or more compounds according to claim 1 and one or more customary extenders and/or surfactants.

13. A product comprising a compound according to claim 1, for protecting the propagation material of plants, optionally for protecting seed.
